SEQUENCE LISTING (3 sequences)

SEQ ID NO: 1 - DNA, 21 bp, Artificial Sequence
  Description of Artificial Sequence Synthetic oligonucleotide
ggccagugccaaguuauautt

SEQ ID NO: 2 - DNA, 21 bp, Artificial Sequence
  Description of Artificial Sequence Synthetic oligonucleotide
gaagagauuuggaaugauutt

SEQ ID NO: 3 - DNA, 21 bp, Artificial Sequence
  Description of Artificial Sequence Synthetic oligonucleotide
auuagacucuucauggaaatt

(19) United States
(12) Patent Application Publication
(10) Pub. No.: US 2011/0124649 A1
(43) Pub. Date: May 26, 2011

(54) INHIBITORS OF HUMAN METHIONINE AMINOPEPTIDASE 1 AND METHODS OF TREATING DISORDERS

(75) Inventors: Jun O. Liu, Clarksville, MD (US); Xiaoyi Hu, San Diego, CA (US); Xiaochun Chen, Baltimore, MD (US); Dawei Ma, Shanghai (CN); Shridhar Bhat, Cockeysville, MD (US)

(73) Assignee: THE JOHNS HOPKINS UNIVERSITY, Baltimore, MD (US)

(21) Appl. No.: 12/742,208

(22) PCT Filed: Nov. 10, 2008
(86) PCT No.: PCT/US2008/012664

§ 371 Date: Jan. 24, 2011

Related U.S. Application Data
(60) Provisional application No. 61/002,578, filed on Nov. 9, 2007.

(51) Int. Cl. A61K 31/53; C07D 401/04; A61K 31/506; A61P 35/00; A61K 31/517; A61K 31/519

(52) U.S. Cl. 514/245; 544/328; 514/256; 544/284; 514/266.21; 514/252.17; 544/249; 514/267; 544/212

(57) ABSTRACT

Described herein are novel pyrimidine-pyridine compounds, methods of inhibiting methionine aminopeptidase and treating disorders (or symptoms thereof) associated with methionine aminopeptidase, wherein a compound of the invention is administered to a subject.

### RELATED APPLICATIONS

This application claims the benefit of U.S. Provisional Application Serial No. 61/002,578, filed Nov. 9, 2007. The entire contents of the provisional application are incorporated herein by this reference.

### GOVERNMENT SUPPORT

This work described herein was supported by a grant from the National Cancer Institute (NCl) (Grant No. CA78743). Therefore, the U.S. Government may have certain rights in the invention.

### BACKGROUND OF THE INVENTION

Protein synthesis is initiated with a methionine residue in eukaryotic cells, or a formylated methionine in prokaryotes, mitochondria and chloroplasts. For a large subset of proteins, the initiator methionine is cotranslationally removed prior to further post-translational modification. The proteolytic removal of N-terminal methionine is catalyzed by a family of enzymes known as methionine aminopeptidases (MetAPs). The functions of these enzymes are evolutionally conserved and essential, as demonstrated by the lethal phenotype of the map null mutant in bacteria. Although only one MetAP gene is present in the genome of most, but not all, prokaryotes, at least two types of MetAPs, type I and type II, are known in eukaryotic cells. In budding yeast Saccharomyces cerevisiae, deletion of either ScMetAP1 or ScMetAP2 resulted in a slow-growth phenotype compared to the wild type strain, whereas the double mutant is non-viable, indicating the redundant yet essential functions of both types of MetAP (Chang, Y. H., et al. (1992) J. Biol. Chem. 267, 8007-8011; Li, X. & Chang, Y. H. (1995) Proc. Natl. Acad. Sci. U.S.A 92, 12357-12361). In multi-cellular organisms, MetAP2 has been shown to be essential for the proliferation and development of specific tissues (Boxem, M., et al. (2004) FEBS Lett. 576, 245-250; Cutforth, T. & Gaul, U. (1999) Mech. Dev. 82, 23-28).

Human MetAP2 has been identified as the primary target of the fumagillin family of natural products that potently inhibit angiogenesis (Griffith, E. C., et al. (1997) Chem. Biol. 4, 461-471; Sin, N., et al. (1997) Proc. Natl. Acad. Sci. U.S.A 94, 6099-6103). A synthetic analog of fumagillin, TNP-470 with higher potency and lower toxicity, has entered clinical trials for a variety of cancers (Ingber, D., et al. (1990) Nature 348, 555-557; Satchi-Fainaro, R., et al. (2005) Cancer Cell 7, 251-261). Much evidence now exists supporting the notion that HsMetAP2 plays an important role in endothelial cell proliferation and is likely to mediate inhibition of endothelial cells by fumagillin and related analogs (Griffith, E. C., et al. (1997) Chem. Biol. 4, 461-471; Sin, N., et al. (1997) Proc. Natl. Acad. Sci. U.S.A 94, 6099-6103; Yeh, J. R., et al. (2006) Proc. Natl. Acad. Sci. U.S.A 103, 10379-10384).

In contrast to HsMetAP2, little is known about the physiological function of human MetAP1, although genetic studies in yeast have suggested a more dominant role for ScMetAP1, as evidenced by the more severe growth defect observed in ScMetAP1 knockout strain than that in ScMetAP2 knockout strain (Chen, S., et al. (2002) Arch. Biochem. Biophys. 398, 87-93). There has also been circumstantial evidence implicating a role of HsMetAP1 in tumor cell proliferation. Using a proteomics-based approach, both human MetAPs were identified as the binding targets of bengamides, a class of marine natural products that inhibit tumor growth in vitro and in vivo (Towbin, H., et al. (2003) J. Biol. Chem. 278, 52964-52971). Recently, pyridinyl pyrimidines have also been identified as non-selective inhibitors for MetAPs, and inhibit the proliferation of tumor cell lines (Hu, X., et al. (2006) Angew. Chem. Int. Ed Engl. 45, 3772-3775). Since most tumor cell lines are refractory to the fumagillin family of HsMetAP2 inhibitors due likely to the defects in p53 pathway, the anti-proliferative effects of bengamides and pyridinyl pyrimidines could have arisen from inhibition of HsMetAP1. These studies suggested an important function of MetAP1 in human cell proliferation. In spite of all the previous studies, however, the physiological function of HsMetAP1 has remained largely unknown.

At least two approaches can be taken, chemical and genetic, to assess the function of human MetAP1 in cell proliferation. In the chemical approach, small chemical compounds could be identified that selectively inhibit the enzymatic activity of HsMetAP1 over HsMetAP2. These compounds would then be used to assess the consequence of inhibition of HsMetAP1 on cell proliferation. Pyridine-2-carboxylic acid-amide derivatives and pyrimidine-pyridine compounds have been previously reported to inhibit both the bacterial and yeast MetAP1 (Luo, Q. L., et al. (2003) J. Med. Chem. 46, 2631-2640; Li, J. Y., et al. (2004) Biochemistry 43, 7892-7898). Certain compounds inhibited HsMetAP1 in cells and blocked proliferation of tumor cell lines. Unlike fumagillin, which arrests cell cycle at G1/S phase, certain compounds caused a significant cell cycle delay during G2/M phase. For the genetic approach, gene-specific silencing of HsMetAP1 also led to a delay in G2/M phase cell cycle progression, corroborating the observations with the specific chemical inhibitors of HsMetAP1. Together, these findings suggested a pivotal role of HsMetAP1 in cell division, which can be exploited in the development of anticancer agents.

It is thus an object of the invention to provide novel compounds that treat cancer and other disorders related to human MetAP activity.

### SUMMARY OF THE INVENTION

In one aspect, the invention provides a compound of formula (I):

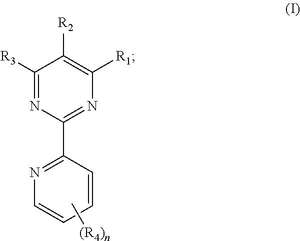

or a pharmaceutically acceptable salt thereof,

wherein,

R₁ is NR_{A}R_{A}, OR_{A}, SR_{A}, optionally substituted heterocyclic, optionally substituted cycloalkyl, or hal;

each R_{A} is independently H, an optionally substituted alkyl, or an optionally substituted aralkyl;

R₂ is H, an optionally substituted alkyl, cyano, nitro, azido, or hal;

R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

or R₂ and R₃ can be taken together to form an optionally substituted aryl;

each R₄ is independently an optionally substituted alkyl or hal; and

n is 0, 1, 2, 3, or 4.

In another aspect, the invention provides a composition comprising any of the compounds described herein, and an additional therapeutic agent.

In one aspect, the invention provides a method of treating a disease or disorder associated with methionine aminopeptidase in a subject, the method comprising the step of administering to the subject an effective amount of a compound of formula VI:

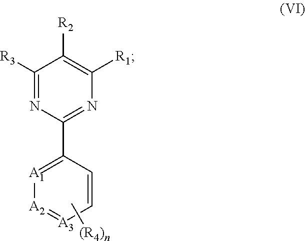

or a pharmaceutically acceptable salt thereof,

wherein,

any one of A₁, A₂, or A₃ is independently CH, CR₄, or N;

R₁ is NR_{A}R_{A}, NHR_{A}, OR_{A}, SR_{A}, optionally substituted heteroaryl, optionally substituted heterocyclic, optionally substituted cycloalkyl, or hal;

each R_{A} is independently H, an optionally substituted alkyl, an optionally substituted aryl, an optionally substituted aralkyl, an optionally substituted heteroaryl, or optionally substituted heterocyclic;

R₂ is H, an optionally substituted alkyl, an optionally substituted alkoxy, cyano, nitro, azido, or halo;

R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

or R₂ and R₃ can be taken together to form an optionally substituted aryl;

each R₄ is independently an optionally substituted alkyl or hal; and

n is 0, 1, 2, 3, or 4.

In another aspect, the invention provides a method of treating a disease or disorder associated with methionine aminopeptidase in a subject, wherein the subject is identified as being in need of a hMetAP1 inhibitor, the method comprising the step of administering to the subject an effective amount of a compound of formula VI:

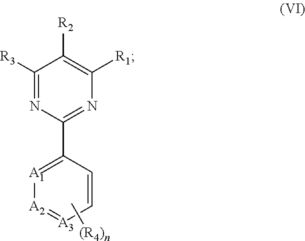

or a pharmaceutically acceptable salt thereof,

wherein,

any one of A₁, A₂, or A₃ is independently CH, CR₄, or N;

R₁ is NR_{A}R_{A}, NHR_{A}, OR_{A}, SR_{A}, optionally substituted heteroaryl, optionally substituted heterocyclic, optionally substituted cycloalkyl, or hal;

each R_{A} is independently H, an optionally substituted alkyl, an optionally substituted aryl, an optionally substituted aralkyl, an optionally substituted heteroaryl, or optionally substituted heterocyclic;

R₂ is H, an optionally substituted alkyl, an optionally substituted alkoxy, cyano, nitro, azido, or halo;

R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

or R₂ and R₃ can be taken together to form an optionally substituted aryl;

each R₄ is independently an optionally substituted alkyl or hal; and

n is 0, 1, 2, 3, or 4.

In one aspect, the invention provides a method of modulating methionine aminopeptidase in a subject, the method comprising the step of administering to the subject an effective amount of a compound identified in a screening assay.

In another aspect, the invention provides a method of modulating methionine aminopeptidase in a subject, the method comprising the step of administering to the subject an effective amount of a compound of formula VI:

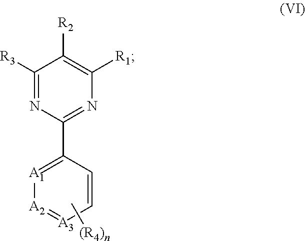

or a pharmaceutically acceptable salt thereof,

wherein,

any one of A₁, A₂, or A₃ is independently CH, CR₄, or N;

R₁ is NR_{A}R_{A}, NHR_{A}, OR_{A}, SR_{A}, optionally substituted heteroaryl, optionally substituted heterocyclic, optionally substituted cycloalkyl, or hal;

each R_{A} is independently H, an optionally substituted alkyl, an optionally substituted aryl, an optionally substituted aralkyl, an optionally substituted heteroaryl, or optionally substituted heterocyclic;

R₂ is H, an optionally substituted alkyl, an optionally substituted alkoxy, cyano, nitro, azido, or halo;

R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

or R₂ and R₃ can be taken together to form an optionally substituted aryl;

each R₄ is independently an optionally substituted alkyl or hal; and

n is 0, 1, 2, 3, or 4.

In other aspects, the invention provides a method of selectively modulating hMetAP1 in a subject, the method comprising the step of administering to the subject an effective amount of a compound identified in a screening assay.

In another aspect, the invention provides a method of treating tumor, cancer growth, or neoplasia in a subject, the method comprising the step of administering to the subject an effective amount of a compound of formula VI:

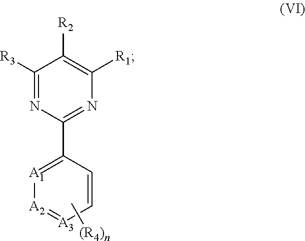

or a pharmaceutically acceptable salt thereof,

wherein,

any one of A₁, A₂, or A₃ is independently CH, CR₄, or N;

R₁ is NR_{A}R_{A}, NHR_{A}, OR_{A}, SR_{A}, optionally substituted heteroaryl, optionally substituted heterocyclic, optionally substituted cycloalkyl, or hal;

each R_{A} is independently H, an optionally substituted alkyl, an optionally substituted aryl, an optionally substituted aralkyl, an optionally substituted heteroaryl, or optionally substituted heterocyclic;

R₂ is H, an optionally substituted alkyl, an optionally substituted alkoxy, cyano, nitro, azido, or halo;

R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

or R₂ and R₃ can be taken together to form an optionally substituted aryl;

each R₄ is independently an optionally substituted alkyl or hal; and

n is 0, 1, 2, 3, or 4;

wherein the compound inhibits hMetAP1 to thereby treat the tumor, cancer growth, or neoplasia.

### BRIEF DESCRIPTION OF THE DRAWINGS

FIG. 1 provides biological data of various pyrimidine-pyridine compounds of the invention.

FIG. 2 provides biological data of various Pyridine-2-carboxylic acid-amide compounds of the invention, structure of pyridine-2-carboxylic acid inhibitors, and their inhibition of HsMetAP1, HsMetAP2 and cell proliferations. Results from Cobalt (II) supplied enzymatic assay were shown. Each experiment was conducted in triplicate with error bars represent ±one SD.

FIG. 3 provides data regarding cells overexpressing targeted HsMetAP1 resisted compound 1 from FIG. 2, in a cell proliferation assay. Each experiment was conducted in triplicate with error bars represent ±one SD

FIG. 4. Inhibition of MetAP by compound 1 from FIG. 2. SDS-PAGE Western blot analysis of HeLa cells exposed to compound 1 from FIG. 2 at the indicated concentrations for 24 h. The membrane was probed with a monoclonal antibody specific for the methionylated 14-3-3γ (upper panel) and tubulin (lower panel), respectively.

FIG. 5. Function of HsMetAP1 is required for accurate cell cycle progression through G2/M phase. A. FACS cell cycle analysis for un-synchronized HeLa cells treated with 1 from FIG. 2 for 24 h. B. Synchronized HeLa cells showed delayed G2/M progression in the presence of compound 1 from FIG. 2. Double thymidine synchronized cells were released for 6 h, 9 h and 12 h, respectively, before collected for FACS analysis. C. Western blot analysis from HeLa cells treated with respective siRNA duplexes for 48 hours. Blots were sequentially probed with anti-HsMetAP1, HsMetAP2 and 14-3-3γ proteins, β-actin is the gel-loading control. D. HsMetAP1 siRNA duplexes delayed cell cycle progression during G2/M phase. Synchronized HeLa cells were harvested for FACS analysis at different time points from double-thymidine release.

FIG. 6. Inhibition of HsMetAP1 resulted in delayed degradation of cyclin B protein. A. A significant delay of cyclin B1 degradation in the presence of 1 from FIG. 2 is shown by western blot. Cell lysates were harvested at different time points from double-thymidine release. B. RT-PCR analysis of cyclin B1 mRNA level was carried out from total RNA isolated at different time points with appropriate primers specific for cyclin B1 and β-actin.

FIG. 7. HsMetAP1 inhibition induces cellular apoptosis. A. Ethidium-bromide-stained genomic DNA isolated from JurKat T cells in the absence (−) and presence (+) of 10 μM compound 1 from FIG. 2, respectively, after 16 hours treatment. M, marker. B. Western blot analysis of protein lysate isolated from vehicle- and compound 1 from FIG. 2-treated JurKat T cells for 24 hours. Blots were sequentially probed with antibodies specific to PARP, procaspase-3 and active caspase 3. Arrow indicated the release of the 89 kDa fragment from PARP protein. β-actin is the gel-loading control.

### DETAILED DESCRIPTION OF THE INVENTION

### I. Definitions

Listed below are definitions of various terms used to describe this invention. These definitions apply to the terms as they are used throughout this specification and claims, unless otherwise limited in specific instances, either individually or as part of a larger group.

The term “alkyl,” as used herein, refers to saturated, straight- or branched-chain hydrocarbon radicals containing, in certain embodiments, between one and six, or one and eight carbon atoms, respectively. Examples of alkyl radicals include, but are not limited to, methyl, ethyl, propyl, isopropyl, n-butyl, tert-butyl, neopentyl, n-hexyl heptyl, octyl radicals.

The term “alkenyl,” as used herein, denote a monovalent group derived from a hydrocarbon moiety containing, in certain embodiments, from two to six, or two to eight carbon atoms having at least one carbon-carbon double bond. The double bond may or may not be the point of attachment to another group. Alkenyl groups include, but are not limited to, for example, ethenyl, propenyl, butenyl, 1-methyl-2-buten-1-yl, heptenyl, octenyl and the like.

The term “alkynyl,” as used herein, denote a monovalent group derived from a hydrocarbon moiety containing, in certain embodiments, from two to six, or two to eight carbon atoms having at least one carbon-carbon triple bond. The alkynyl group may or may not be the point of attachment to another group. Representative alkynyl groups include, but are not limited to, for example, ethynyl, 1-propynyl, 1-butynyl, heptynyl, octynyl and the like.

The term “cycloalkyl” or “carbocyclic” are used interchangeably, and as used herein, denotes a monovalent group derived from a monocyclic or polycyclic saturated carbocyclic ring compound. Examples include, but are not limited to, cyclopropyl, cyclobutyl, cyclopentyl, cyclohexyl, cyclopentyl and cyclooctyl; bicyclo [2.2.1] heptyl, and bicyclo [2.2.2] octyl. Also contemplated are a monovalent group derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, idenyl and the like.

The term “aralkyl,” as used herein, refers to an alkyl residue attached to an aryl ring. Examples include, but are not limited to, benzyl, phenethyl and the like.

The term “heteroaryl,” as used herein, refers to a mono- or poly-cyclic (e.g., bi-, or tri-cyclic or more) fused or non-fused, radical or ring system having at least one aromatic ring, having from five to ten ring atoms of which one ring atoms is selected from S, O and N; zero, one or two ring atoms are additional heteroatoms independently selected from S, O and N; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “heteroaralkyl,” as used herein, refers to an alkyl residue residue attached to a heteroaryl ring. Examples include, but are not limited to, pyridinylmethyl, pyrimidinylethyl and the like.

The term “heterocycloalkyl,” as used herein, refers to a non-aromatic 3-, 4-, 5-, 6- or 7-membered ring or a bi- or tri-cyclic group fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, (iv) the nitrogen heteroatom may optionally be quaternized, and (iv) any of the above rings may be fused to a benzene ring. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The terms “optionally substituted”, “optionally substituted alkyl,” “optionally substituted “optionally substituted alkenyl,” “optionally substituted alkynyl”, “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocycloalkyl,” and any other optionally substituted group as used herein, refer to groups that are substituted or unsubstituted by independent replacement of one, two, or three or more of the hydrogen atoms thereon with substituents including, but not limited to: —F, —Cl, —Br, —I, —OH, protected hydroxy, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -dihetero arylamino, —O—C₁-C₁₂-alkyl, —O—C₂-C₁₂-alkenyl, —O—C₂-C₁₂-alkenyl, —O—C₃-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkenyl, —CONH—C₃-C₁₂-cycloalkyl, —CONH-aryl, —CONH-heteroaryl, —CONH-heterocycloalkyl, —OCO₂—C₁-C₁₂-alkyl, —OCO₂—C₂-C₁₂-alkenyl, —OCO₂—C₂-C₁₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkenyl, —OCONH—C₃-C₁₂-cycloalkyl, —OCONH— aryl, —OCONH— heteroaryl, —OCONH— heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂-aryl, —NHCO₂— heteroaryl, —NHCO₂— heterocycloalkyl, —NHC(O)NH₂, —NHC(O)NH—C₁-C₁₂-alkyl, —NHC(O)NH—C₂-C₁₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, —NHC(O)NH-heterocycloalkyl, NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NHC(NH)NH-aryl, —NHC(NH)NH-heteroaryl, —NHC(NH)NH-heterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkenyl, —C(NH)NH—C₃-C₁₂-cycloalkyl, —C(NH)NH-aryl, —C(NH)NH-heteroaryl, —C(NH)NH-heterocycloalkyl, —S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH— aryl, —SO₂NH— heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S-heterocycloalkyl, or methylthiomethyl.

It is understood that the aryls, heteroaryls, alkyls, and the like can be further substituted. In accordance with the invention, any of the aryls, substituted aryls, heteroaryls and substituted heteroaryls described herein, can be any aromatic group. Aromatic groups can be substituted or unsubstituted.

It is understood that any alkyl, alkenyl, alkynyl, cycloalkyl and cycloalkenyl moiety described herein can also be an aliphatic group, an alicyclic group or a heterocyclic group. An “aliphatic group” is non-aromatic moiety that may contain any combination of carbon atoms, hydrogen atoms, halogen atoms, oxygen, nitrogen or other atoms, and optionally contain one or more units of unsaturation, e.g., double and/or triple bonds. An aliphatic group may be straight chained, branched or cyclic and preferably contains between about 1 and about 24 carbon atoms, more typically between about 1 and about 12 carbon atoms. In addition to aliphatic hydrocarbon groups, aliphatic groups include, for example, polyalkoxyalkyls, such as polyalkylene glycols, polyamines, and polyimines, for example. Such aliphatic groups may be further substituted. It is understood that aliphatic groups may be used in place of the alkyl, alkenyl, alkynyl, alkylene, alkenylene, and alkynylene groups described herein.

The terms “hal,” “halo” and “halogen,” are used interchangeably, and as used herein, refer to an atom selected from fluorine, chlorine, bromine and iodine.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

As used herein, the term “pharmaceutically acceptable salt” refers to those salts of the compounds formed by the process of the present invention which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al. describes pharmaceutically acceptable salts in detail in J. Pharmaceutical Sciences, 66: 1-19 (1977). The salts can be prepared in situ during the final isolation and purification of the compounds of the invention, or separately by reacting the free base function with a suitable organic acid. Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

The term “effective amount” is used throughout the specification to describe concentrations or amounts of compounds according to the present invention which may be used to produce a favorable change in the disease or condition treated, whether that change is a remission, a decrease in growth or size of cancer, tumor or other growth, a favorable physiological result including the clearing up of skin or tissue, or the like, depending upon the disease or condition treated.

As used herein, the terms “prevent,” “preventing,” “prevention,” and the like refer to reducing the probability of developing a disorder or condition in a subject, who does not have, but is at risk of or susceptible to developing a disorder or condition.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

The term “tumor” is used to describe an abnormal growth in tissue which occurs when cellular proliferation is more rapid than normal tissue and continues to grow after the stimuli that initated the new growth cease. Tumors generally exhibit partial or complete lack of structural organization and functional coordination with the normal tissue, and usually form a distinct mass of tissue which may be benign (benign tumor) or malignant (carcinoma). Tumors tend to be highly vascularized.

The term “cancer” is used as a general term herein to describe malignant tumors or carcinoma. These malignant tumors may invade surrounding tissues, may metastasize to several sites and are likely to recur after attempted removal and to cause death of the patient unless adequately treated. As used herein, the terms carcinoma and cancer are subsumed under the term tumor. Methods of treating tumors and/or cancer according to the present invention comprise administering to a patient in need thereof an effective amount of one or compounds according to the present invention.

### II. Compounds of the Invention

In one aspect, the invention provides a compound of formula (I):

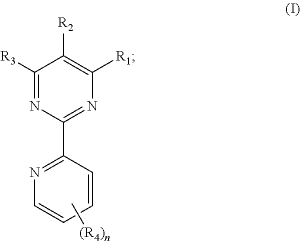

or a pharmaceutically acceptable salt thereof,

wherein,

R₁ is NR_{A}R_{A}, OR_{A}, SR_{A}, optionally substituted heterocyclic, optionally substituted cycloalkyl, or hal;

each R_{A} is independently H, an optionally substituted alkyl, or an optionally substituted aralkyl;

R₂ is H, an optionally substituted alkyl, cyano, nitro, azido, or hal;

R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

or R₂ and R₃ can be taken together to form an optionally substituted aryl;

each R₄ is independently an optionally substituted alkyl or hal; and

n is 0, 1, 2, 3, or 4.

In one embodiment, the invention provides a compound of formula I, wherein R₁ is NR_{A}R_{A}, OR_{A}, or SR_{A}.

In another embodiment, the invention provides a compound of formula I, wherein R₁ is NH₂, NH—CH₂—CH₂—R_{B}, NH—CH₂—CH₂—NH—R_{B}, O—CH₂—CH₂—R_{B}, S—CH₂—CH₂—R_{B}, S—CH₂—R_{B},

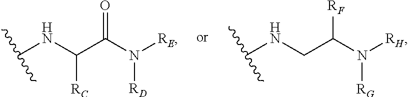

each of which may be optionally substituted.

In a further embodiment, each R_{B} is independently an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl.

In certain embodiments, R_{C} is an optionally substituted alkyl or an optionally substituted aralkyl.

In other embodiments, R_{D} and R_{E} are each independently H, an optionally substituted alkyl, an optionally substituted aralkyl, or an optionally substituted hetero-aralkyl.

In certain embodiments, R_{F} is an optionally substituted aryl, an optionally substituted heteroaryl, an optionally substituted cycloalkyl, or an optionally substituted heterocycloalkyl.

In other embodiments, R_{G} is H or an optionally substituted alkyl.

In one embodiment, R_{H} is X(CH₂)ₘY; and m is 1, 2, 3, 4, or 5.

In a further embodiment, X is C(O), S(O)ₚ, or absent; and p is 0, 1, or 2.

In another further embodiment, Y is an optionally substituted cycloalkyl, an optionally substituted heterocycloalkyl, an optionally substituted aryl, or an optionally substituted heteroaryl.

In one embodiment, R₁ is hal.

In another embodiment, R₁ is optionally substituted heterocyclic or optionally substituted cycloalkyl.

In a further embodiment, R₁ is piperazinyl, piperidinyl, morpholinyl, pyrrolidinyl, pyrrolinyl, 2H-pyrrolyl, tetrahydrofuranyl, tetrahydroisoquinolinyl, tetrahydroquinolinyl, cyclopropyl, cyclobutyl, cyclopentyl, cyclohexyl, cycloheptyl, adamantyl, cyclooctyl, [3.3.0]bicyclooctane, [4.3.0]bicyclononane, [4.4.0]bicyclodecane, or [2.2.2]bicyclooctane; each of which may be optionally substituted.

In one embodiment, the invention provides a compound of formula (II):

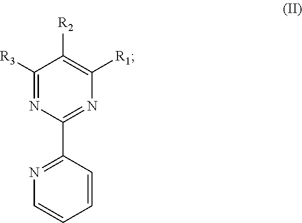

or a pharmaceutically acceptable salt thereof,

wherein,

R₁ is NR_{A}R_{A}, OR_{A}, SR_{A}, or hal;

each R_{A} is independently H, an optionally substituted alkyl, or an optionally substituted aralkyl;

R₂ is H, an optionally substituted alkyl, cyano, nitro, azido, or halo; and

R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl.

In one embodiment, R₂ is H, an optionally substituted alkyl, cyano, nitro, azido, or halo; and R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl.

In another embodiment, R₂ is H, an optionally substituted alkyl, cyano, or halo; and R₃ is an optionally substituted alkyl, an optionally substituted haloalkyl, or an optionally substituted aryl.

In still another embodiment, R₁ is NH₂, NH—CH₂—CH₂—R_{B}, NH—CH₂—CH₂—NH—R_{B}, O—CH₂—CH₂—R_{B}, S—CH₂—CH₂—R_{B}, S—CH₂—R_{B}, each of which may be optionally substituted.

In yet another embodiment, each R_{B} is an optionally substituted aryl or an optionally substituted heteroaryl.

In certain embodiments, the invention provides a compound selected from:

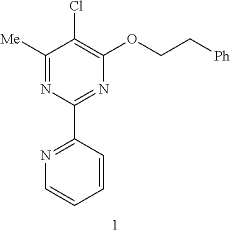

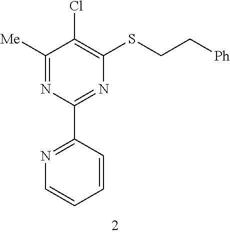

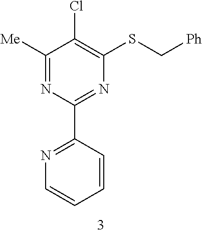

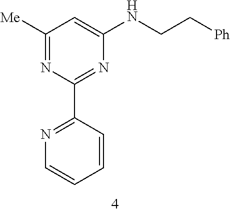

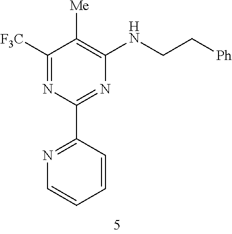

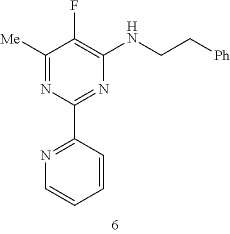

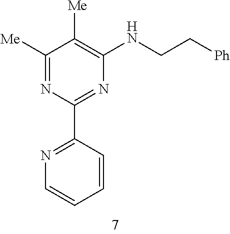

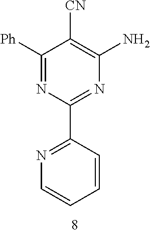

In one embodiment, the invention provides a compound of formula (III):

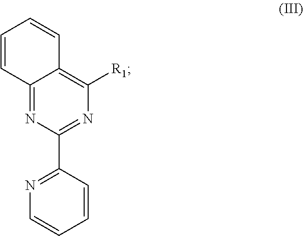

or a pharmaceutically acceptable salt thereof,

wherein,

R₁ is NR_{A}R_{A}, OR_{A}, SR_{A}, optionally substituted heterocyclic, optionally substituted cycloalkyl, or hal;

each R_{A} is independently H, an optionally substituted alkyl, or an optionally substituted aralkyl.

In certain embodiments, R₁ is hal, NH₂, NH—CH₂—CH₂—R_{B}, NH—CH₂—CH₂—NH—R_{B}, optionally substituted heterocyclic, or optionally substituted cycloalkyl, each of which may be optionally substituted.

In another embodiment, each R_{B} is an optionally substituted aryl or an optionally substituted heteroaryl.

In other embodiments, the optionally substituted heterocyclic is piperazinyl, piperidinyl, or morpholinyl; each of which may be optionally substituted.

In certain embodiments, the invention provides for a compound selected from:

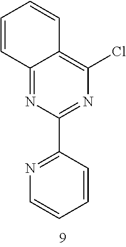

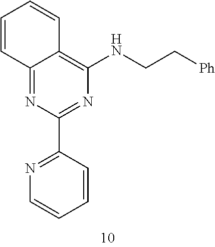

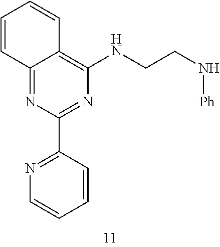

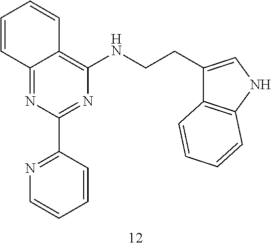

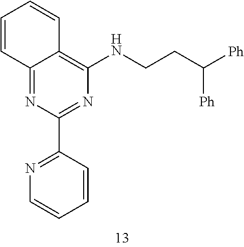

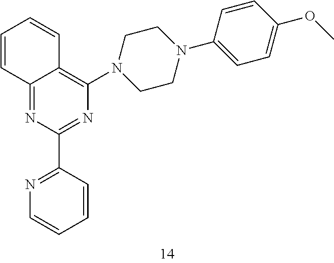

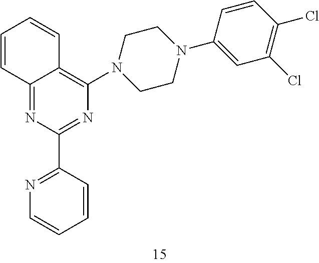

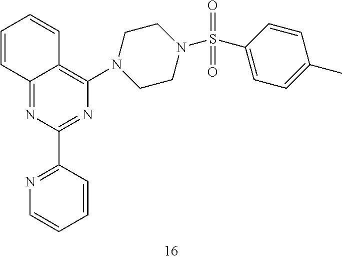

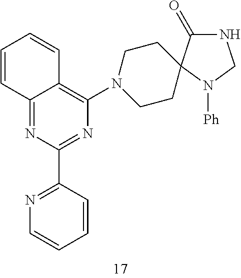

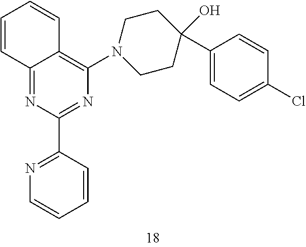

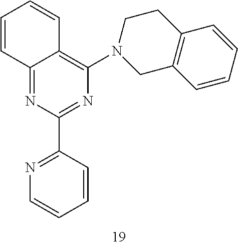

In one embodiment, the invention provides for a compound of formula (IV):

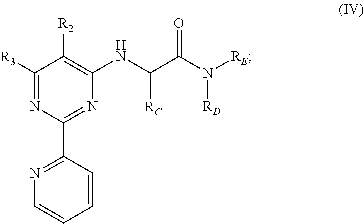

or a pharmaceutically acceptable salt thereof,

wherein,

R_{C} is an optionally substituted alkyl or an optionally substituted aralkyl;

R_{D} and R_{E} are each independently H, an optionally substituted alkyl, an optionally substituted aralkyl, or an optionally substituted hetero-aralkyl;

R₂ is H, an optionally substituted alkyl, cyano, nitro, azido, or halo; and

R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl.

In certain embodiments, Rc is methyl, ethyl, n-propyl, i-propyl, n-butyl, i-butyl, t-butyl, or benzyl, each of which may be optionally substituted.

In other embodiments, R_{D} is H or an optionally substituted alkyl.

In other embodiments, R_{E} is H, methyl, ethyl, or benzyl, each of which is optionally substituted.

In a further embodiment, R_{E} is substituted with hal, alkyl, haloalkyl, alkoxyl, haloalkoxy, phenyl, furanyl, nitro, cyano, or nitrile, each of which may be optionally substituted.

In certain embodiments, R₂ is H, an optionally substituted alkyl, or halo.

In other embodiments, R₃ is an optionally substituted alkyl.

In certain embodiments, the invention provides a compound selected from:

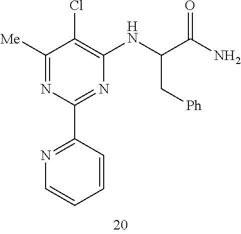

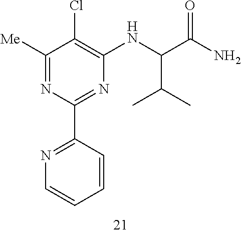

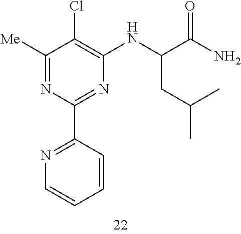

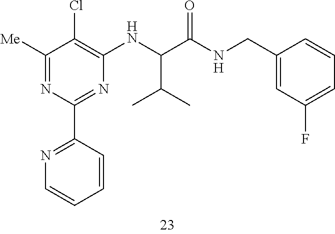

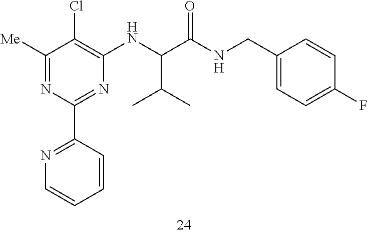

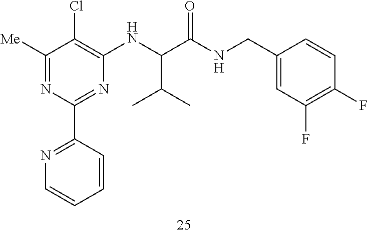

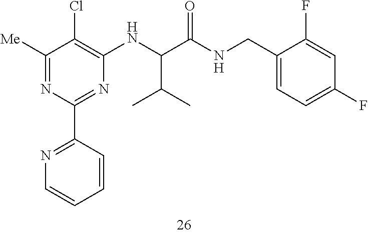

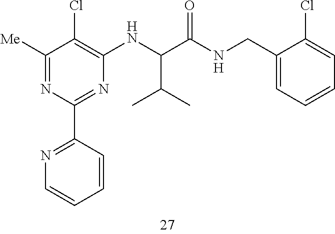

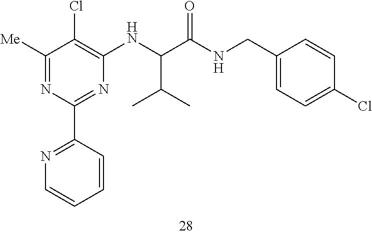

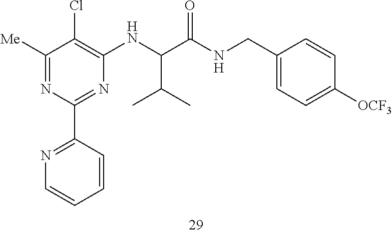

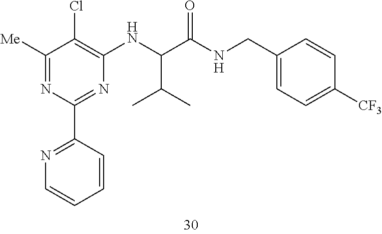

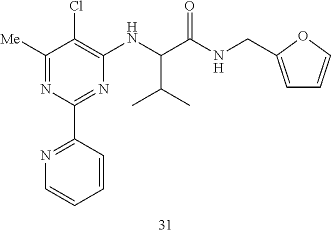

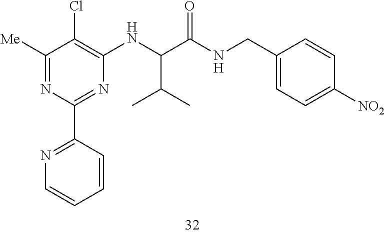

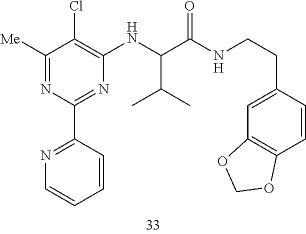

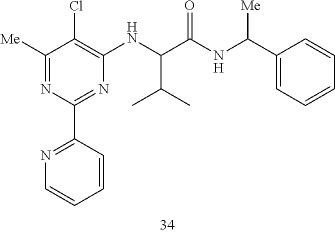

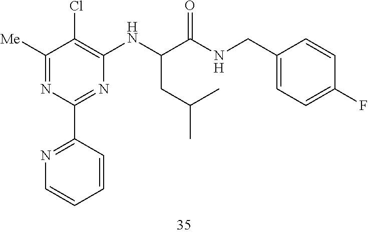

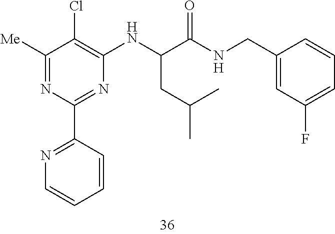

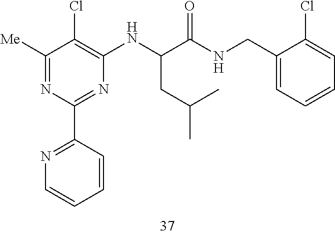

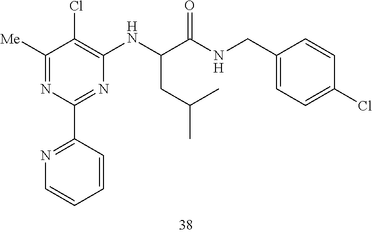

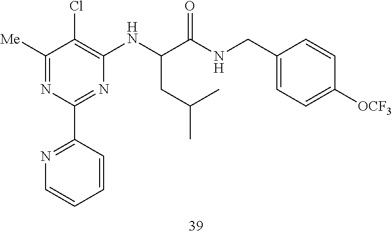

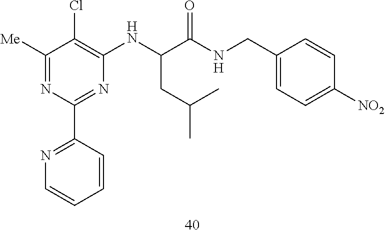

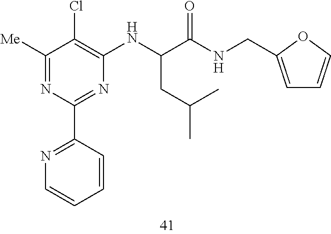

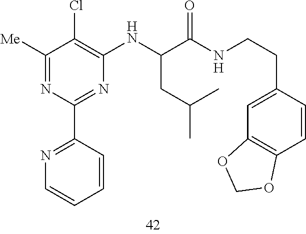

In one embodiment, the invention provides a compound of formula (V):

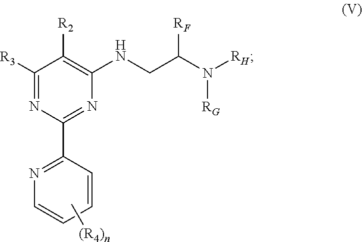

or a pharmaceutically acceptable salt thereof,

wherein,

R_{F} is an optionally substituted aryl, an optionally substituted heteroaryl, an optionally substituted cycloalkyl, or an optionally substituted heterocycloalkyl;

R_{G} is H or an optionally substituted alkyl;

R_{H} is X(CH₂)ₘY; and m is 1, 2, 3, 4, or 5;

X is C(O), S(O)ₚ, or absent; and p is 0, 1, or 2;

Y is an optionally substituted cycloalkyl, an optionally substituted heterocycloalkyl, an optionally substituted aryl, or an optionally substituted heteroaryl;

R₂ is H, an optionally substituted alkyl, cyano, nitro, azido, or halo;

R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

each R₄ is independently an optionally substituted alkyl or hal; and

n is 0, 1, 2, 3, or 4.

In certain embodiments, R_{F} is an optionally substituted aryl or an optionally substituted heteroaryl.

In other embodiments, X is C(O).

In a further embodiment, Y is an optionally substituted cycloalkyl or an optionally substituted heterocycloalkyl.

In other embodiments, X is S(O)ₚ, and p is 0, 1, or 2.

In certain embodiments, Y is an optionally substituted cycloalkyl or an optionally substituted heterocycloalkyl.

In another embodiment, X is absent.

In certain embodiments, Y is an optionally substituted cycloalkyl or an optionally substituted heterocycloalkyl.

In still another embodiment, R₂ is H, an optionally substituted alkyl, or halo.

In certain embodiments, R₃ is an optionally substituted alkyl.

In other embodiments, each R₄ is independently an optionally substituted alkyl or hal; and n is 0, 1, or 2.

In certain embodiments, the invention provides a compound selected from:

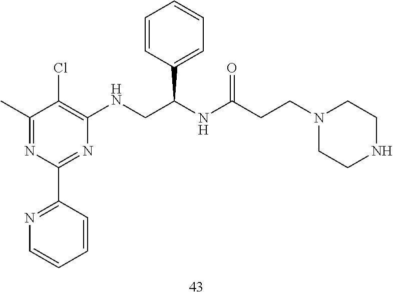

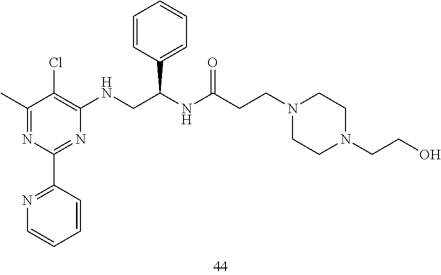

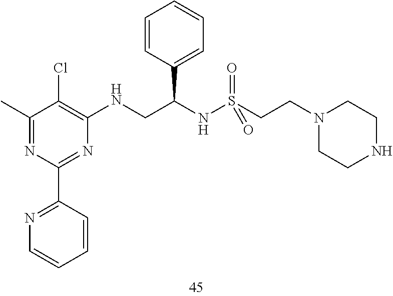

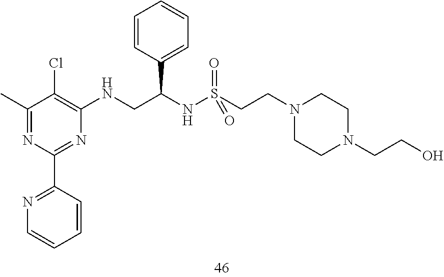

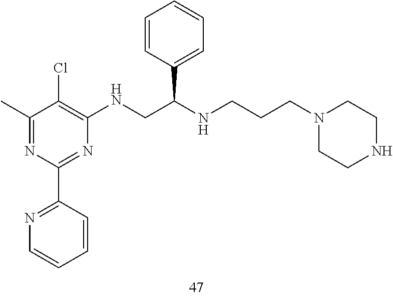

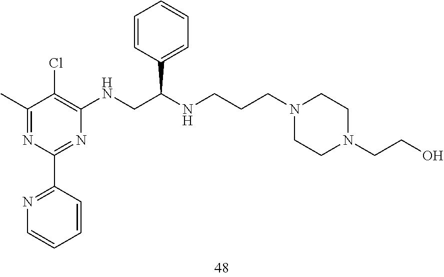

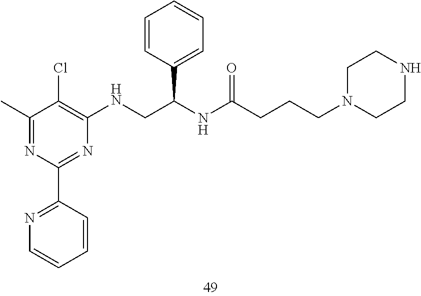

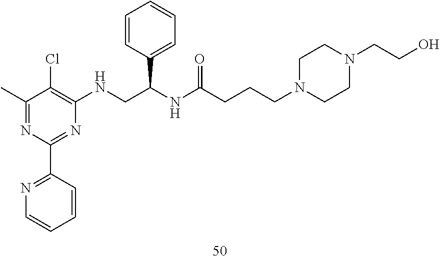

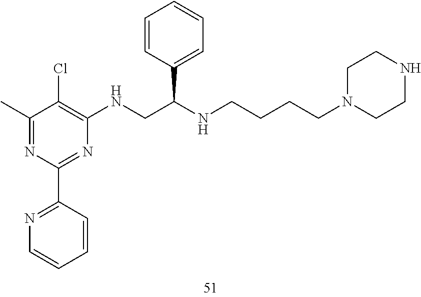

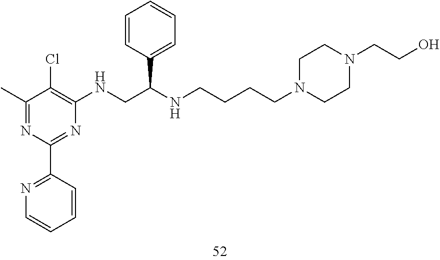

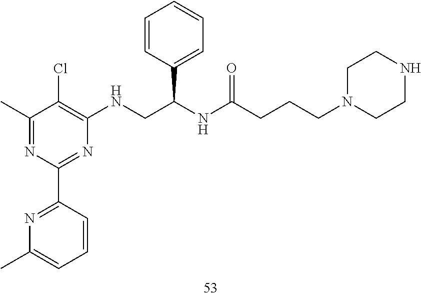

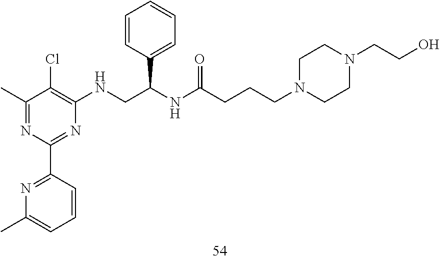

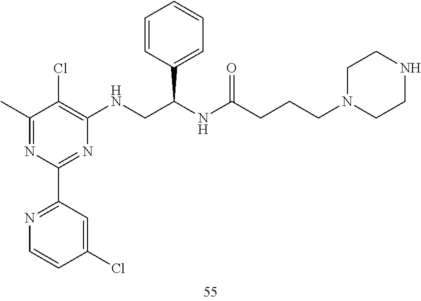

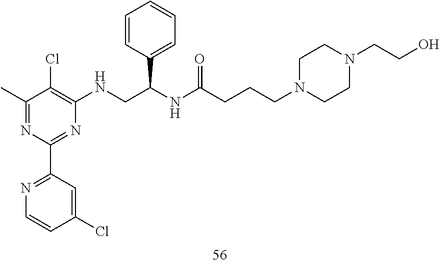

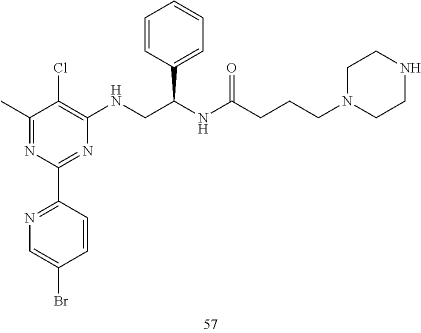

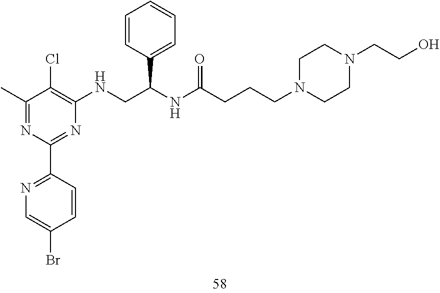

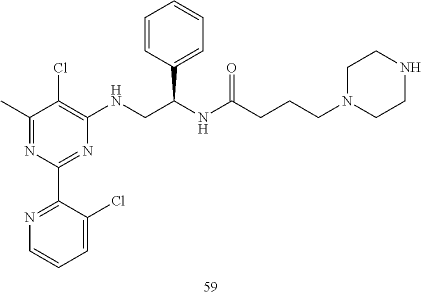

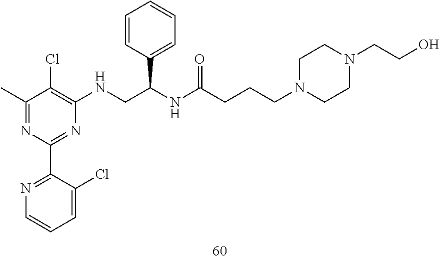

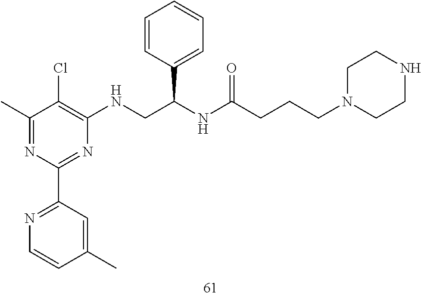

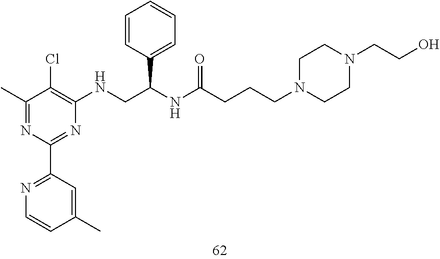

In another aspect, the invention provides a compound selected from

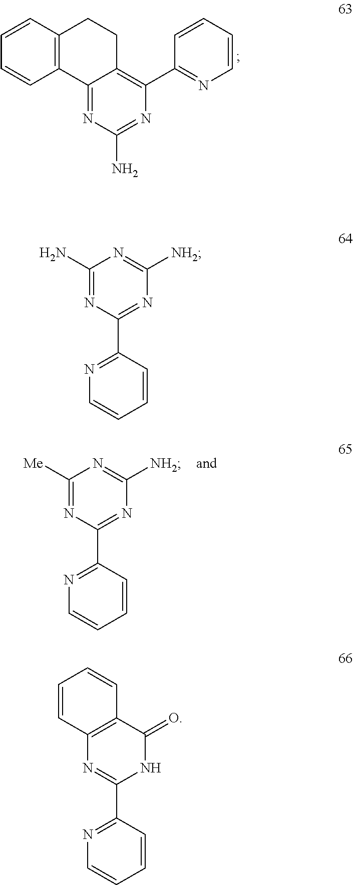

In another aspect, the invention provides a composition comprising any of the compounds described herein, and an additional therapeutic agent.

In one embodiment, the invention provides a composition wherein the additional therapeutic agent is a methionine aminopeptidase-inhibiting compound.

In another embodiment, the additional therapeutic agent is an anticancer compound.

Certain compounds of the present invention may exist in particular geometric, isomeric, or stereoisomeric forms. The present invention contemplates all such compounds, including cis- and trans-isomers, R- and S-enantiomers, racemates and racemic mixtures, single enantiomers, individual diastereomers, diastereomeric mixtures diastereomers, (D)-isomers, (L)-isomers, the racemic mixtures thereof, and other mixtures thereof, as falling within the scope of the invention. The compounds of this invention may also be represented in multiple tautomeric forms, in such instances, the invention expressly includes all tautomeric forms of the compounds described herein (e.g., alkylation of a ring system may result in alkylation at multiple sites, the invention expressly includes all such reaction products). Additional asymmetric carbon atoms may be present in a substituent such as an alkyl group. All such enriched isomers, as well as racemic mixtures thereof, are intended to be included in this invention. All such isomeric forms of such compounds are expressly included in the present invention. All crystal forms of the compounds described herein are expressly included in the present invention.

The compounds described above can be prepared by methods well known in the art, as well as by the synthetic routes disclosed in the examples below.

The chemicals used in the above-described synthetic route may include, for example, solvents, reagents, catalysts, and protecting group and deprotecting group reagents. The methods described above may also additionally include steps, either before or after the steps described specifically herein, to add or remove suitable protecting groups in order to ultimately allow synthesis of the heterocyclic compounds. In addition, various synthetic steps may be performed in an alternate sequence or order to give the desired compounds. Synthetic chemistry transformations and protecting group methodologies (protection and deprotection) useful in synthesizing applicable heterocyclic compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

Correspondingly, the compounds described herein can be made according to methods know in the art, including those in the aforementioned treatises. It is recognized by one of ordinary skill that reaction conditions (e.g., temperature, reaction time, etc.) may be adjusted, which is routine for one of ordinary skill.

As can be appreciated by the skilled artisan, further methods of synthesizing the compounds of the formulae herein will be evident to those of ordinary skill in the art. Additionally, the various synthetic steps may be performed in an alternate sequence or order to give the desired compounds. Synthetic chemistry transformations and protecting group methodologies (protection and deprotection) useful in synthesizing the compounds described herein are known in the art and include, for example, those such as described in R. Larock, Comprehensive Organic Transformations, 2nd. Ed., Wiley-VCH Publishers (1999); T. W. Greene and P. G. M. Wuts, Protective Groups in Organic Synthesis, 3rd. Ed., John Wiley and Sons (1999); L. Fieser and M. Fieser, Fieser and Fieser's Reagents for Organic Synthesis, John Wiley and Sons (1999); and L. Paquette, ed., Encyclopedia of Reagents for Organic Synthesis, John Wiley and Sons (1995), and subsequent editions thereof.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions. The process of converting refers to one or more chemical transformations, which can be performed in situ, or with isolation of intermediate compounds. The transformations can include reacting the starting compounds or intermediates with additional reagents using techniques and protocols known in the art, including those in the references cited herein. Intermediates can be used with or without purification (e.g., filtration, distillation, crystallization, chromatography). Other embodiments relate to the intermediate compounds delineated herein, and their use in the methods (e.g., treatment, synthesis) delineated herein.

### III. Methods of Treatment

In one aspect, the invention provides a method of treating a disease or disorder associated with methionine aminopeptidase in a subject, the method comprising the step of administering to the subject an effective amount of a compound of formula VI:

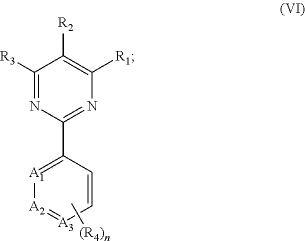

or a pharmaceutically acceptable salt thereof,

wherein,

any one of A₁, A₂, or A₃ is independently CH, CR₄, or N;

R₁ is NR_{A}R_{A}, NHR_{A}, OR_{A}, SR_{A}, optionally substituted heteroaryl, optionally substituted heterocyclic, optionally substituted cycloalkyl, or hal;

each R_{A} is independently H, an optionally substituted alkyl, an optionally substituted aryl, an optionally substituted aralkyl, an optionally substituted heteroaryl, or optionally substituted heterocyclic;

R₂ is H, an optionally substituted alkyl, an optionally substituted alkoxy, cyano, nitro, azido, or halo;

R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

or R₂ and R₃ can be taken together to form an optionally substituted aryl;

each R₄ is independently an optionally substituted alkyl or hal; and

n is 0, 1, 2, 3, or 4.

In another aspect, the invention provides a method of treating a disease or disorder associated with methionine aminopeptidase in a subject, wherein the subject is identified as being in need of a hMetAP1 inhibitor, the method comprising the step of administering to the subject an effective amount of a compound of formula VI:

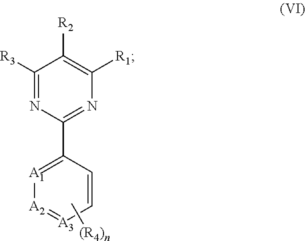

or a pharmaceutically acceptable salt thereof,

wherein,

any one of A₁, A₂, or A₃ is independently CH, CR₄, or N;

R₁ is NR_{A}R_{A}, NHR_{A}, OR_{A}, SR_{A}, optionally substituted heteroaryl, optionally substituted heterocyclic, optionally substituted cycloalkyl, or hal;

each R_{A} is independently H, an optionally substituted alkyl, an optionally substituted aryl, an optionally substituted aralkyl, an optionally substituted heteroaryl, or optionally substituted heterocyclic;

R₂ is H, an optionally substituted alkyl, an optionally substituted alkoxy, cyano, nitro, azido, or halo;

R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

or R₂ and R₃ can be taken together to form an optionally substituted aryl;

each R₄ is independently an optionally substituted alkyl or hal; and

n is 0, 1, 2, 3, or 4.

In certain embodiments, the invention provides a method wherein the methionine aminopeptidase is human type 1 methionine aminopeptidase (hMetAP1).

In a further embodiment, the disease or disorder associated with hMetAP1 is tumor, cancer growth, or neoplasia. Disorders treated by the invention include eye or ocular cancer, rectal cancer, colon cancer, cervical cancer, prostate cancer, breast cancer and bladder cancer, oral cancer, benign and malignant tumors, stomach cancer, liver cancer, pancreatic cancer, lung cancer, corpus uteri, ovary cancer, prostate cancer, testicular cancer, renal cancer, brain/cns cancer, throat cancer, skin melanoma, leukemia, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, mouth/pharynx cancer, esophageal cancer, larynx cancer, lymphoma, neurofibromatosis, tuberous sclerosis, hemangiomas, and lymphangiogenesis.

In one aspect, the invention provides a method of modulating methionine aminopeptidase in a subject, the method comprising the step of administering to the subject an effective amount of a compound identified in a screening assay.

In certain embodiments, the screening assay is selected from MetAP enzyme assay, Double Thymidine synchronization, Cell cycle analysis, and siRNA Transfection.

In another aspect, the invention provides a method of modulating methionine aminopeptidase in a subject, the method comprising the step of administering to the subject an effective amount of a compound of formula VI:

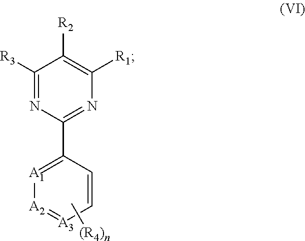

or a pharmaceutically acceptable salt thereof,

wherein,

any one of A₁, A₂, or A₃ is independently CH, CR₄, or N;

R₁ is NR_{A}R_{A}, NHR_{A}, OR_{A}, SR_{A}, optionally substituted heteroaryl, optionally substituted heterocyclic, optionally substituted cycloalkyl, or hal;

each R_{A} is independently H, an optionally substituted alkyl, an optionally substituted aryl, an optionally substituted aralkyl, an optionally substituted heteroaryl, or optionally substituted heterocyclic;

R₂ is H, an optionally substituted alkyl, an optionally substituted alkoxy, cyano, nitro, azido, or halo;

R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

or R₂ and R₃ can be taken together to form an optionally substituted aryl;

each R₄ is independently an optionally substituted alkyl or hal; and

n is 0, 1, 2, 3, or 4.

In one embodiment, the methionine aminopeptidase is hMetAP1.

In a further embodiment, the modulation is inhibition.

In another further embodiment, the compound selectively inhibits hMetAP1 over hMetAP2.

In other aspects, the invention provides a method of selectively modulating hMetAP1 in a subject, the method comprising the step of administering to the subject an effective amount of a compound identified in a screening assay.

In one embodiment, the hMetAP1 inhibitor has a IC₅₀ for inhibiting hMetAP1 less than about 5 micromolar micromolar.

In another aspect, the invention provides a method of treating tumor, cancer growth, or neoplasia in a subject, the method comprising the step of administering to the subject an effective amount of a compound of formula VI:

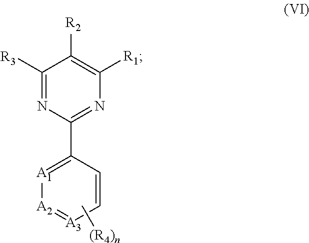

or a pharmaceutically acceptable salt thereof,

wherein,

any one of A₁, A₂, or A₃ is independently CH, CR₄, or N;

R₁ is NR_{A}R_{A}, NHR_{A}, OR_{A}, SR_{A}, optionally substituted heteroaryl, optionally substituted heterocyclic, optionally substituted cycloalkyl, or hal;

each R_{A} is independently H, an optionally substituted alkyl, an optionally substituted aryl, an optionally substituted aralkyl, an optionally substituted heteroaryl, or optionally substituted heterocyclic;

R₂ is H, an optionally substituted alkyl, an optionally substituted alkoxy, cyano, nitro, azido, or halo;

R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;

or R₂ and R₃ can be taken together to form an optionally substituted aryl;

each R₄ is independently an optionally substituted alkyl or hal; and

n is 0, 1, 2, 3, or 4;

wherein the compound inhibits hMetAP1 to thereby treat the tumor, cancer growth, or neoplasia.

In certain embodments, the method further comprises a step of administering an additional therapeutic agent.

In a further embodiment, the additional therapeutic agent is a hMetAP1 inhibiting compound.

In another further embodiment, the additional therapeutic agent is an anticancer compound.

In certain embodiments, the invention provides a method wherein the step of administering the compound comprises administering the compound orally, topically, parentally, intravenously or intramuscularly.

In other embodiments, the invention provides a method wherein the step of administering the compound comprises administering the compound in a dosage of between about 0.1 and 120 mg/kg/day.

In other embodiments, the invention provides a method wherein the step of administering the compound comprises administering the compound in a dosage of less than about 500 mg/day.

In certain embodiments, the subject is a human.

The invention also provides the use of a compound in the manufacture of a medicament for inhibiting hMetAP1 in a patient, wherein the compound is a compound of formula VI.

Diseases or disorders treated, ameliorated or prevented by the instant invention include the following: neoplasia, internal malignancies such as eye or ocular cancer, rectal cancer, colon cancer, cervical cancer, prostate cancer, breast cancer and bladder cancer, benign and malignant tumors, including various cancers such as, anal and oral cancers, stomach, rectal, liver, pancreatic, lung, cervix uteri, corpus uteri, ovary, prostate, testis, renal, mouth/pharynx, esophageal, larynx, kidney, brain/cns (e.g., gliomas), head and neck, throat, skin melanoma, acute lymphocytic leukemia, acute myelogenous leukemia, Ewing's Sarcoma, Kaposi's Sarcoma, basal cell carinoma and squamous cell carcinoma, small cell lung cancer, choriocarcinoma, rhabdomyosarcoma, angiosarcoma, hemangioendothelioma, Wilms Tumor, neuroblastoma, lymphoma, neurofibromatosis, tuberous sclerosis (each of which conditions produces benign tumors of the skin), hemangiomas, lymphangiogenesis, rhabdomyosarcomas, retinoblastoma, osteosarcoma, acoustic neuroma, neurofibroma, trachoma, pyogenic granulomas, and blood-born tumors such as leukemias.

Other disorders treated by the compounds of the invention include any of various acute or chronic neoplastic diseases of the bone marrow in which unrestrained proliferation of white blood cells occurs, usually accompanied by anemia, impaired blood clotting, and enlargement of the lymph nodes, liver, and spleen, psoriasis, acne, rosacea, warts, eczema, neurofibromatosis, Sturge-Weber syndrome, venous ulcers of the skin, tuberous sclerosis, chronic inflammatory disease, arthritis, lupus, scleroderma, diabetic retinopathy, retinopathy of prematurity, corneal graft rejection, neovascular glaucoma and retrolental fibroplasias, epidemic keratoconjunctivitis, vitamin A deficiency, contact lens overwear, atopic keratitis, superior limbic keratitis, pterygium, keratitis sicca, Sjogren's, phylectenulosis, syphilis, Mycobacteria infections, lipid degeneration, chemical burns; bacterial ulcers, fungal ulcers, herpes simplex infections, herpes zoster infections, protozoan infections, Mooren's ulcer, Terrien's marginal degeneration, marginal keratolysis, trauma, rheumatoid arthritis, systemic lupus, polyarteritis, Wegener's sarcoidosis, scleritis, Stevens-Johnson disease, pemphigoid, radial keratotomy, corneal graft rejection, diabetic retinopathy, macular edema, macular degeneration, sickle cell anemia, sarcoid, pseudoxanthoma elasticum, Paget's disease, vein occlusion, artery occlusion, carotid obstructive disease, chronic uveitis/vitritis, Lyme disease, systemic lupus erythematosus, Eales' disease, Behcet's disease, infections causing a retinitis or choroiditis, presumed ocular histoplasmosis, Best's disease, myopia, optic pits, Stargardt's disease, pars planitis, chronic retinal detachment, hyperviscosity syndromes, toxoplasmosis, trauma, post-laser complications, rubeosis (neovascularization of the ankle), diseases caused by the abnormal proliferation of fibrovascular or fibrous tissue including all forms of proliferative vitreoretinopathy, whether or not associated with diabetes, neovascular disease, pannus, diabetic macular edema, vascular retinopathy, retinal degeneration, inflammatory diseases of the retina, proliferative vitreoretinopathy, diseases associated with rubeosis (neovascularization of the ankle), diseases caused by the abnormal proliferation of fibrovascular or fibrous tissue including all forms of proliferative vitreoretinopathy, Crohn's disease and ulcerative colitis, sarcoidosis, osteoarthritis, inflammatory bowel diseases, skin lesions, Osler-Weber-Rendu disease, or hereditary hemorrhagic telangiectasia, osteoarthritis, Sarcoidosis, skin lesions, acquired immune deficiency syndrome, and small bowel obstruction.

The present compounds may be used to treat subjects including animals, and in particular, mammals, including humans, as patients. Thus, humans and other animals, and in particular, mammals, suffering from diseases or disorders related to hMetAP1, can be treated, ameliorated or prevented by administering to the patient an effective amount of one or more of the compounds according to the present invention or its derivative or a pharmaceutically acceptable salt thereof optionally in a pharmaceutically acceptable carrier or diluent, either alone, or in combination with other known pharmaceutical agents (depending upon the disease to be treated). Treatment according to the present invention can also be administered in conjunction with other conventional therapies, e.g., cancer therapy, such as radiation treatment or surgery.

The compounds of the invention may be utilized in combination with at least one known other therapeutic agent, or a pharmaceutically acceptable salt of said agent. Examples of known therapeutic agents which can be used for combination therapy include, but are not limited to, corticosteroids (e.g., cortisone, prodnisone, dexamethasone), non-steroidal anti-inflammatory drugs (NSAIDS) (e.g., ibuprofen, celecoxib, aspirin, indomethicin, naproxen), alkylating agents such as busulfan, cis-platin, mitomycin C, and carboplatin; antimitotic agents such as colchicine, vinblastine, paclitaxel, and docetaxel; topo I inhibitors such as camptothecin and topotecan; topo II inhibitors such as doxorubicin and etoposide; RNA/DNA antimetabolites such as 5-azacytidine, 5-fluorouracil and methotrexate; DNA antimetabolites such as 5-fluoro-2′-deoxy-uridine, ara-C, hydroxyurea and thioguanine; antibodies such as Herceptin® and Rituxan®. Other known anti-cancer agents which can be used for combination therapy include melphalan, chlorambucil, cyclophosamide, ifosfamide, vincristine, mitoguazone, epirubicin, aclarubicin, bleomycin, mitoxantrone, elliptinium, fludarabine, octreotide, retinoic acid, tamoxifen and alanosine.

### IV. Mechanism of Action

HsMetAP1-specific Inhibitors Block Proliferation of Tumor Cell Lines

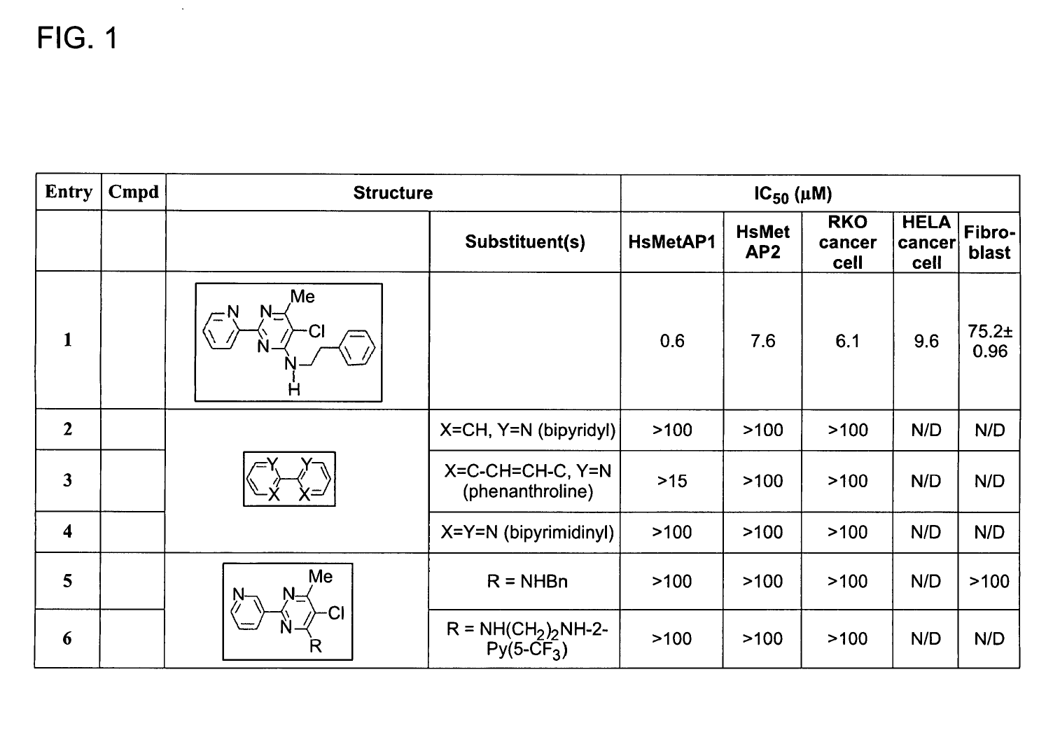

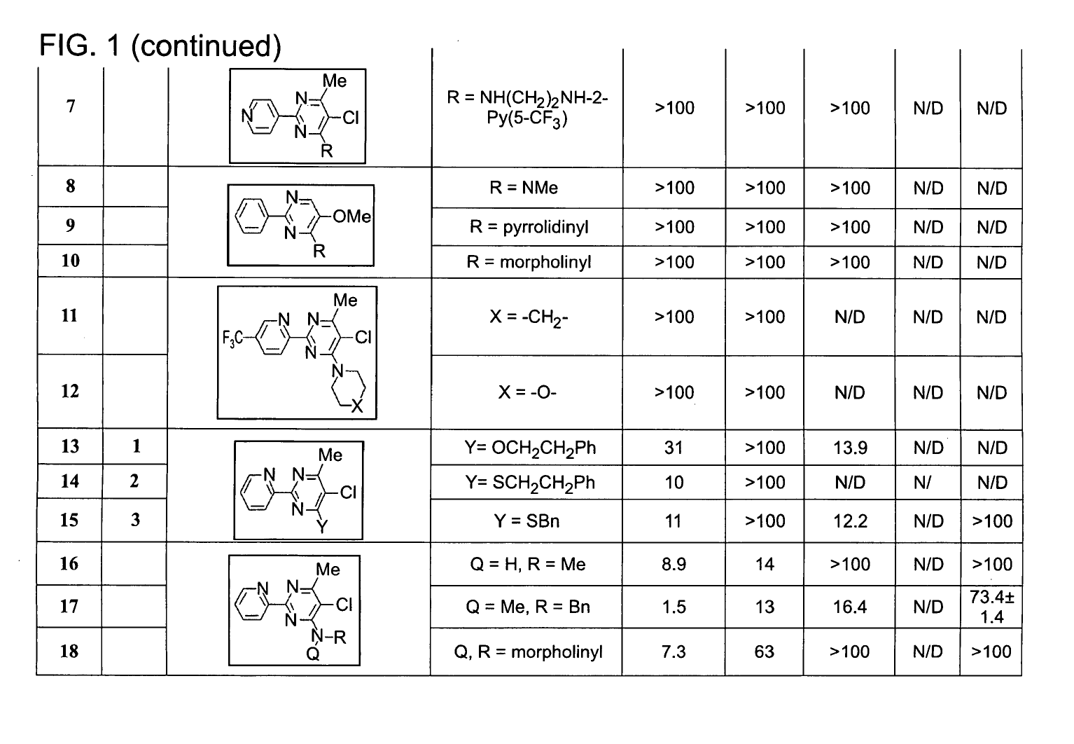

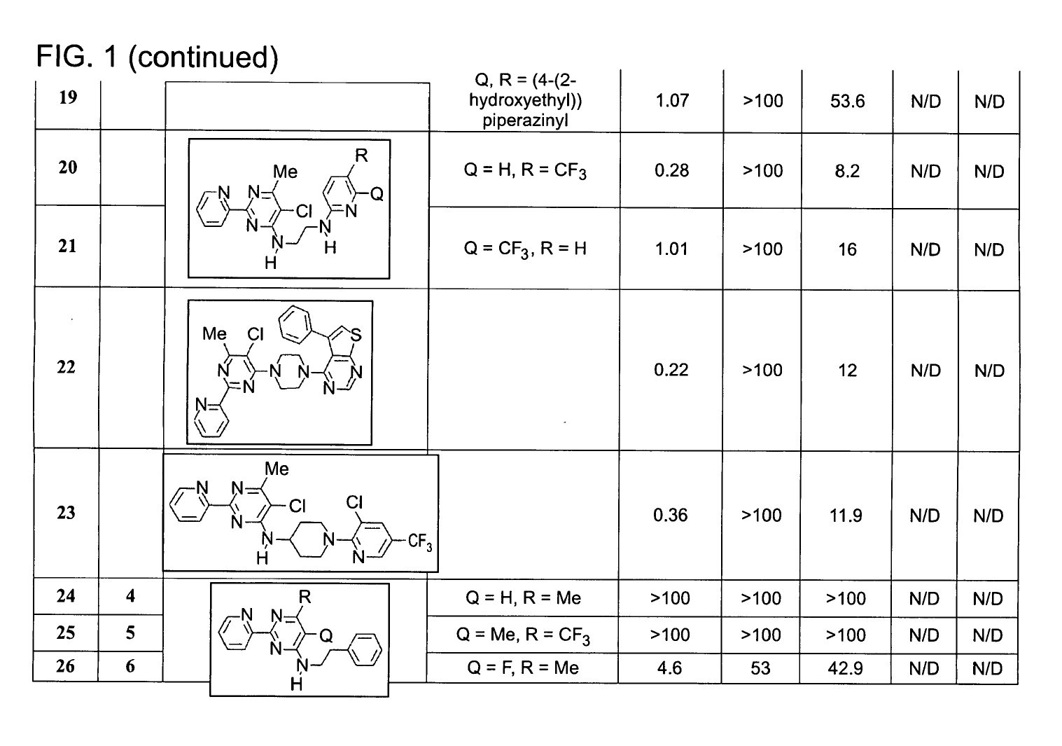

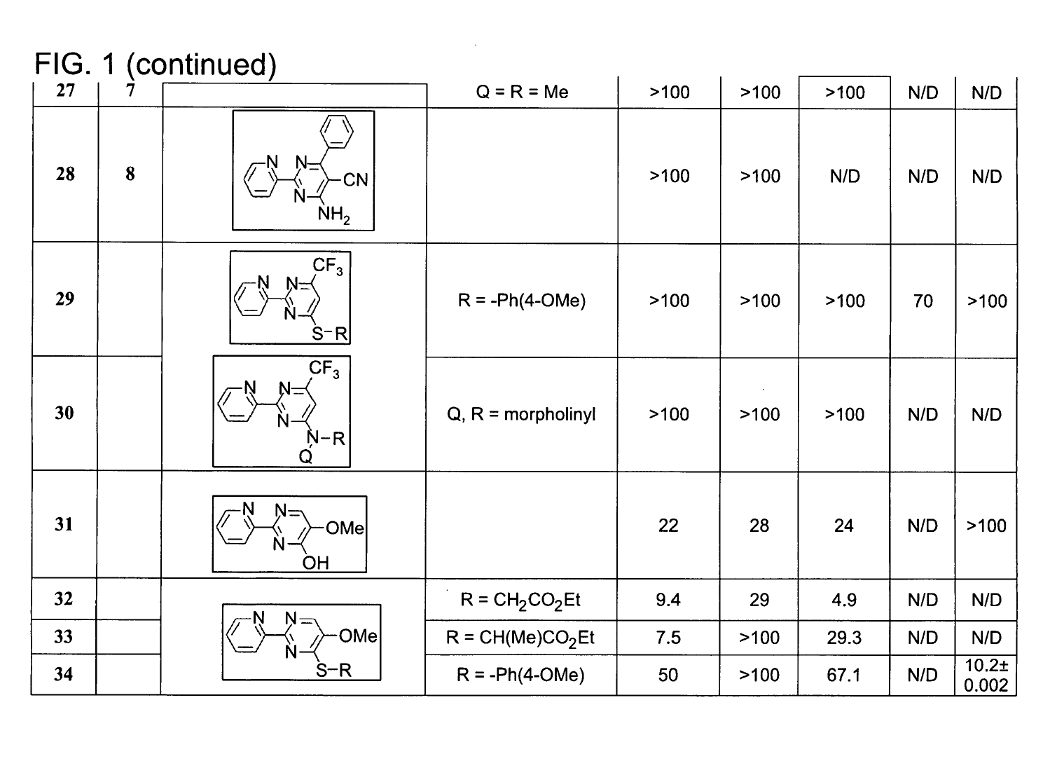

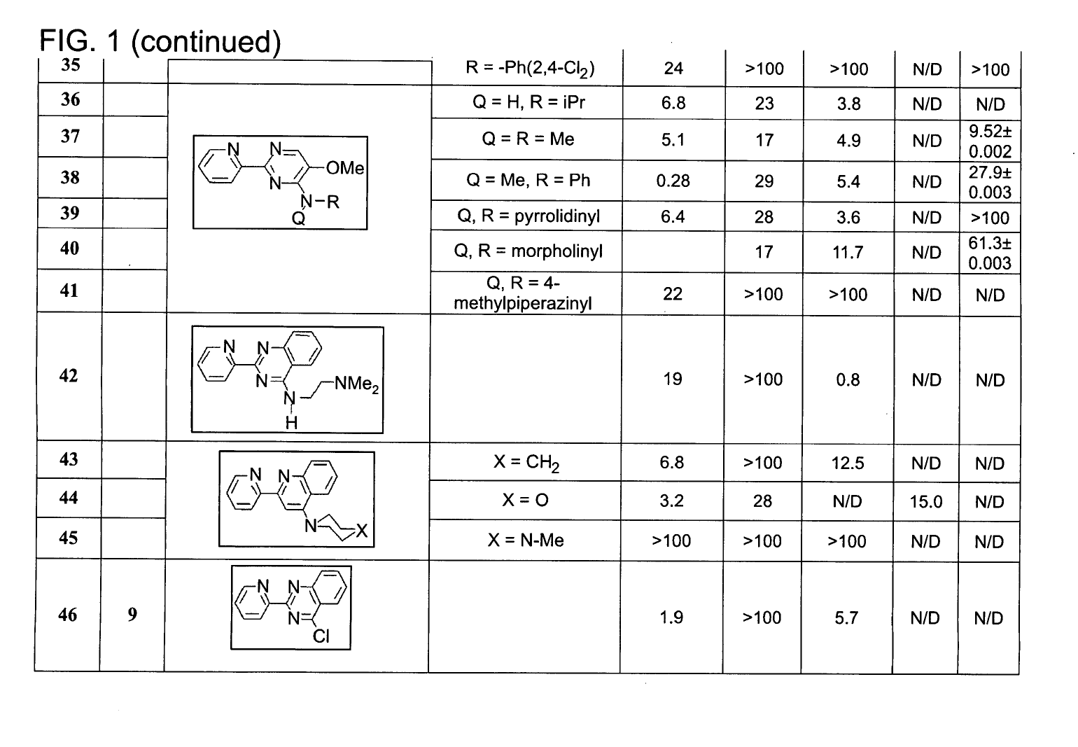

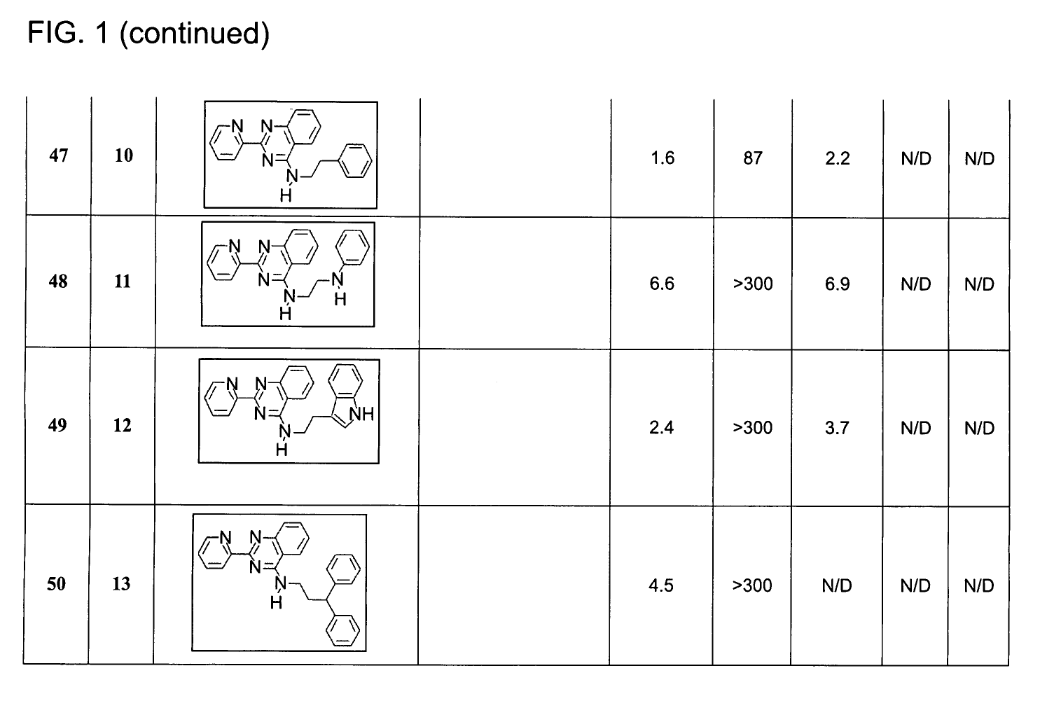

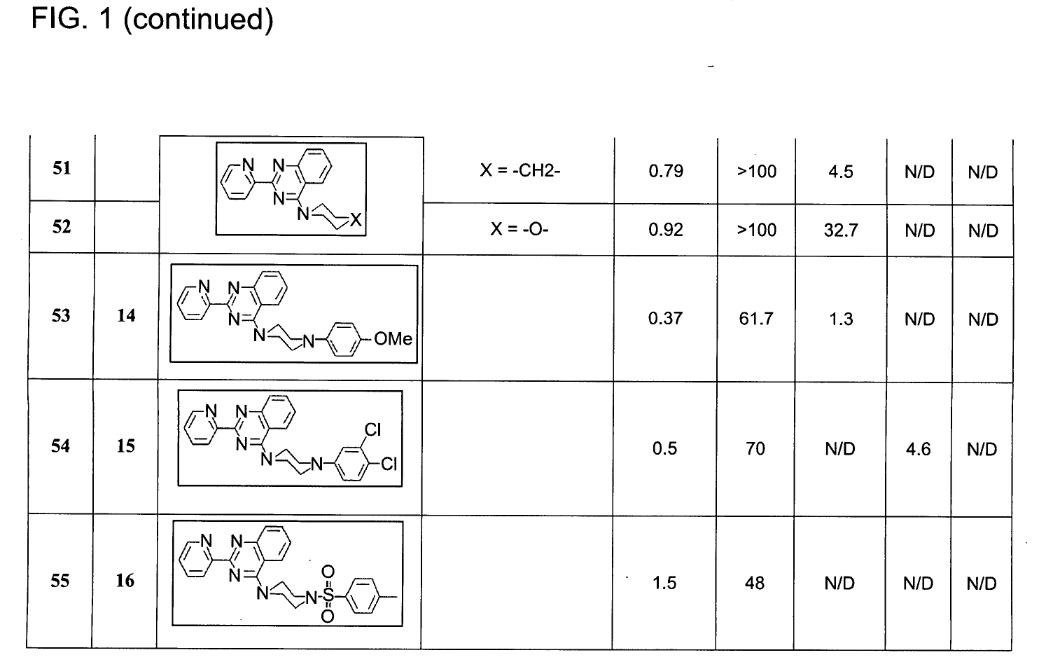

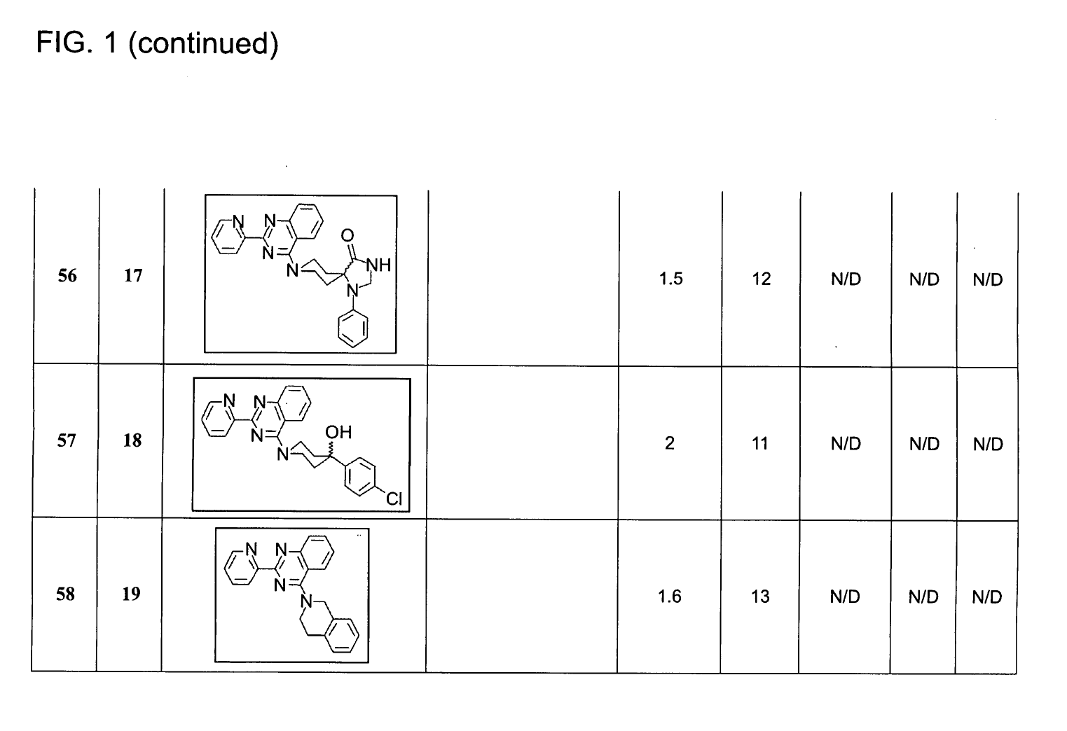

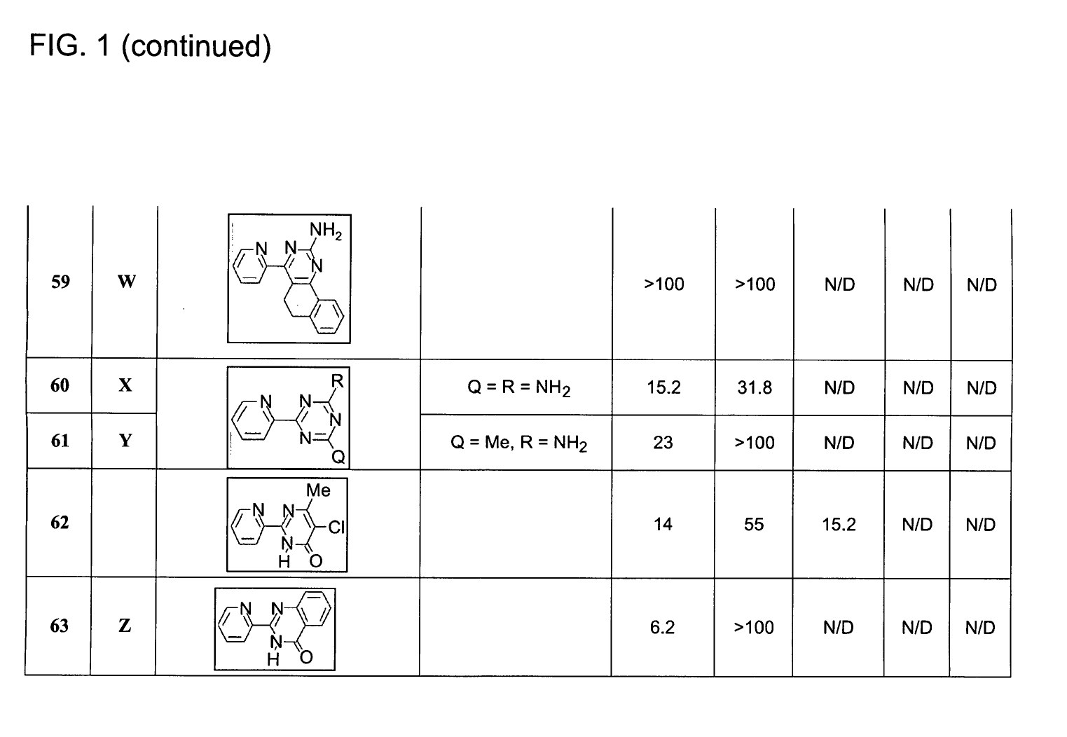

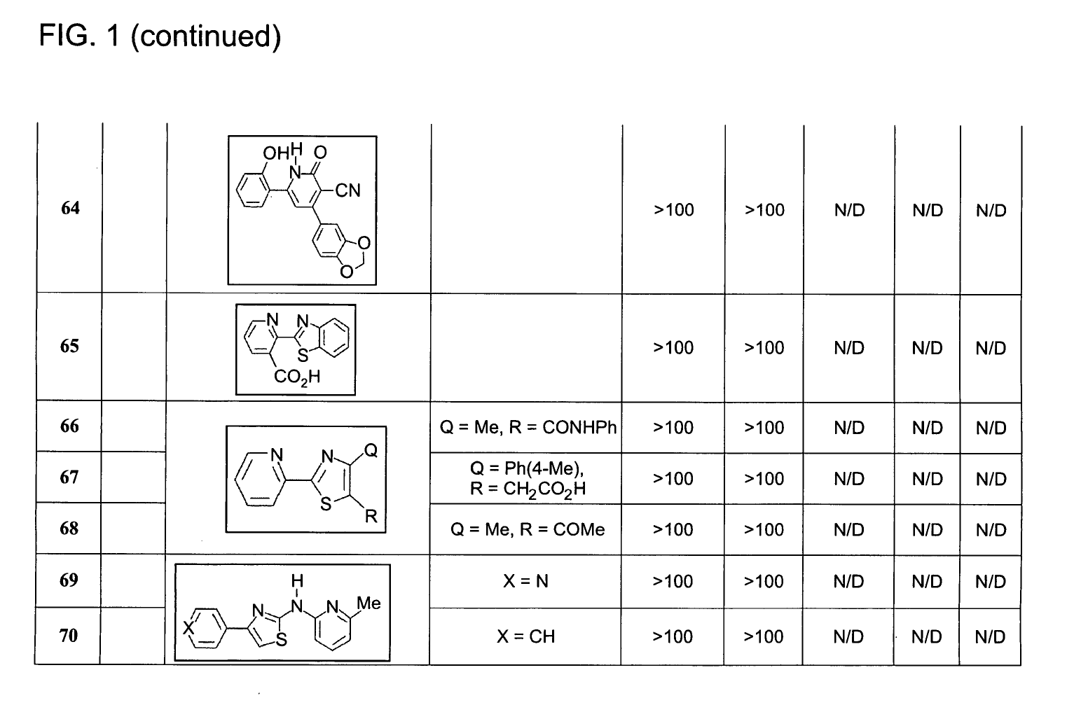

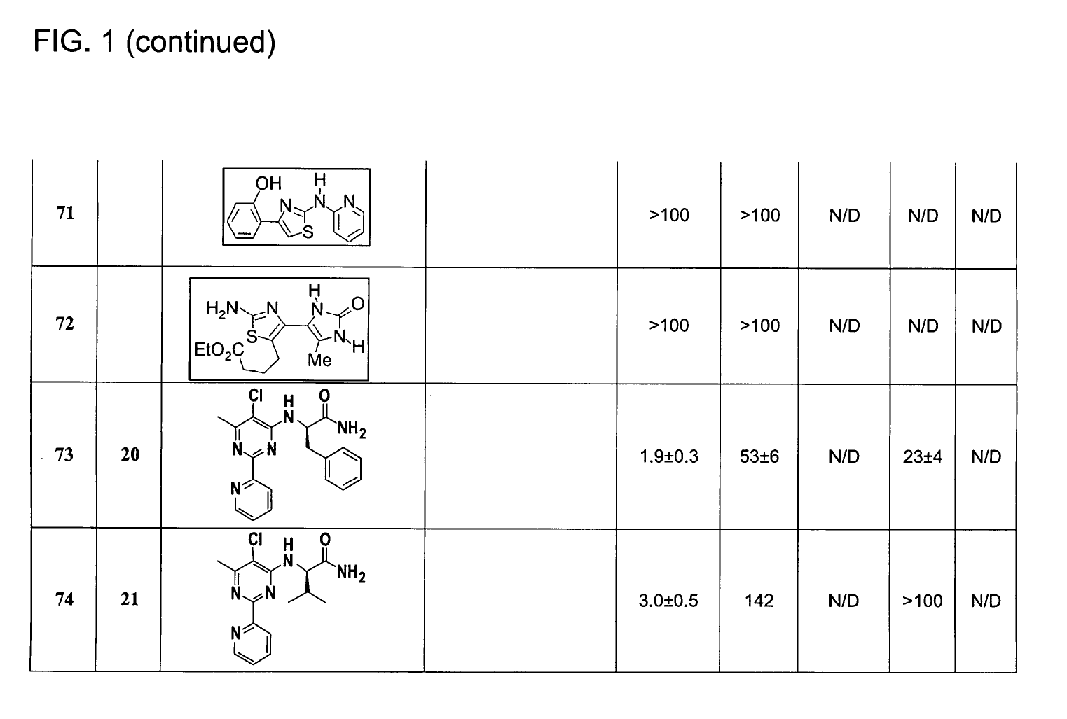

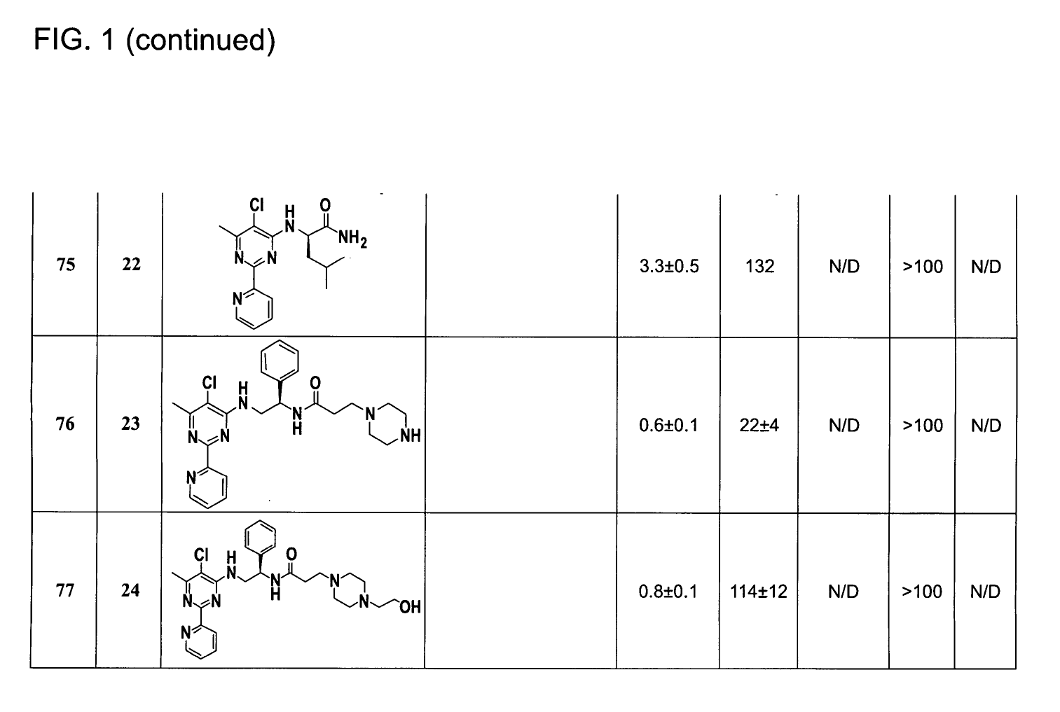

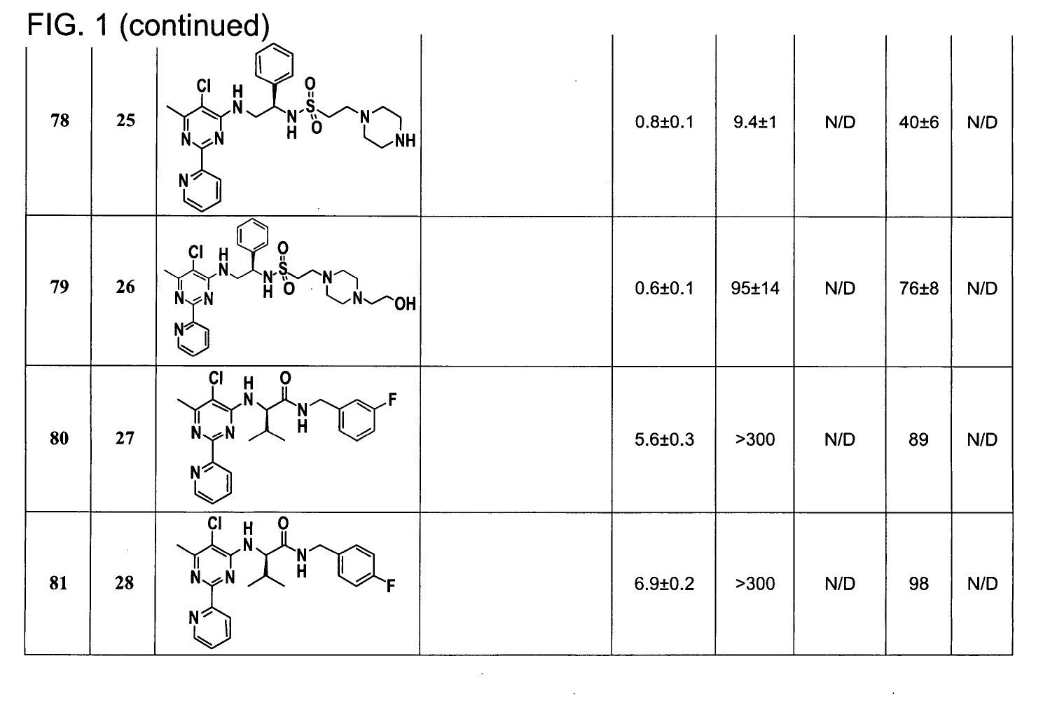

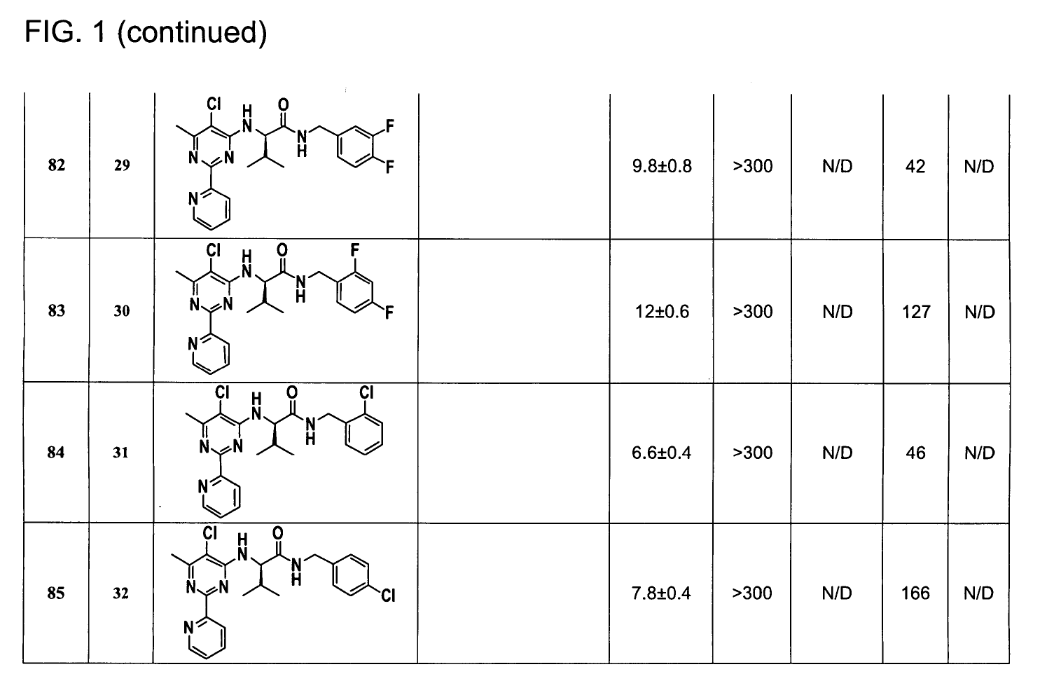

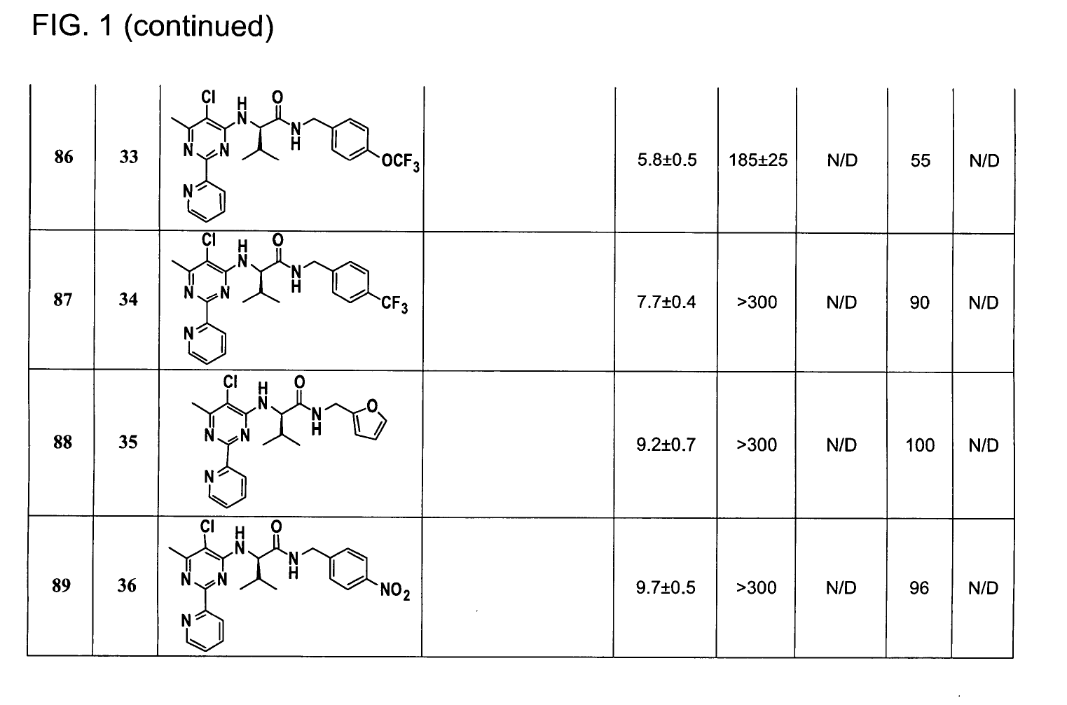

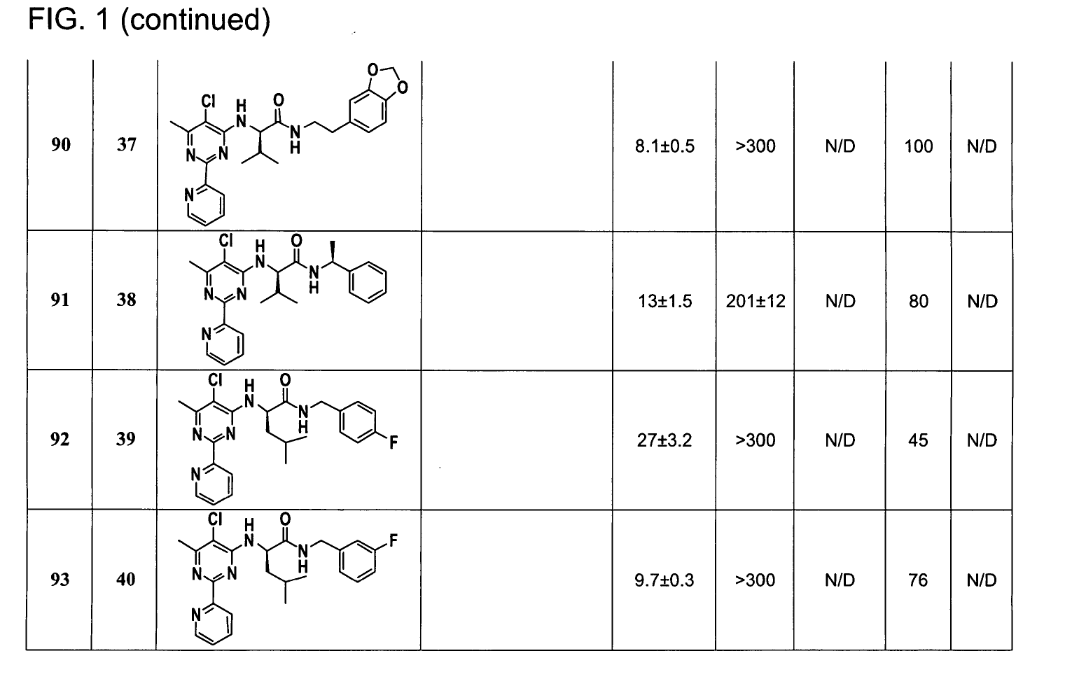

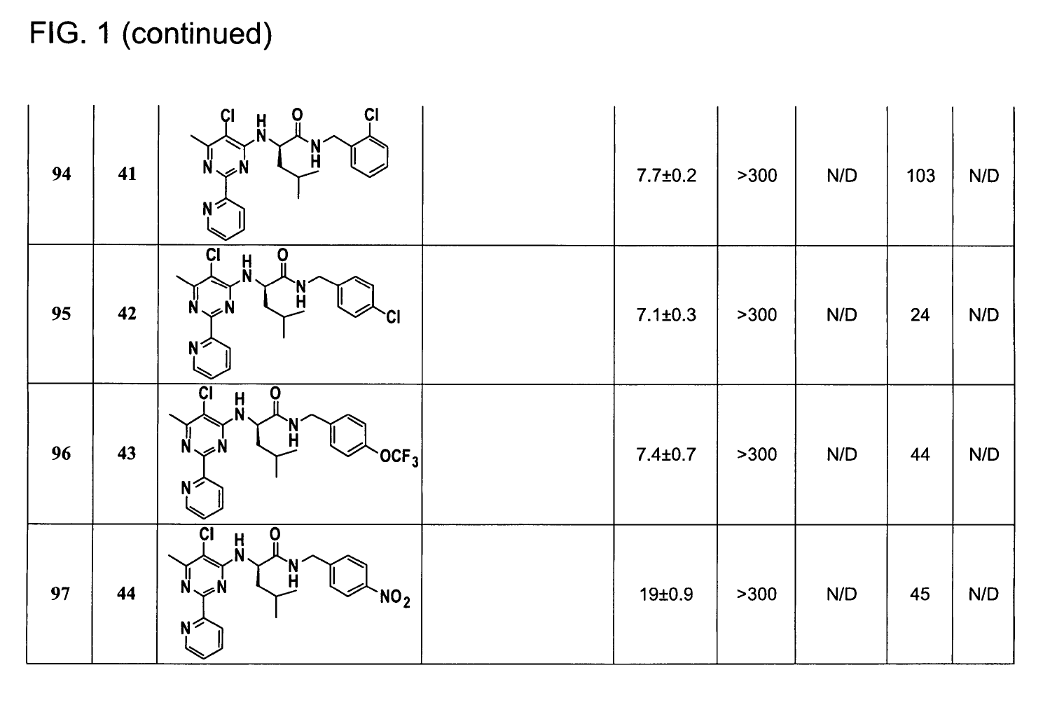

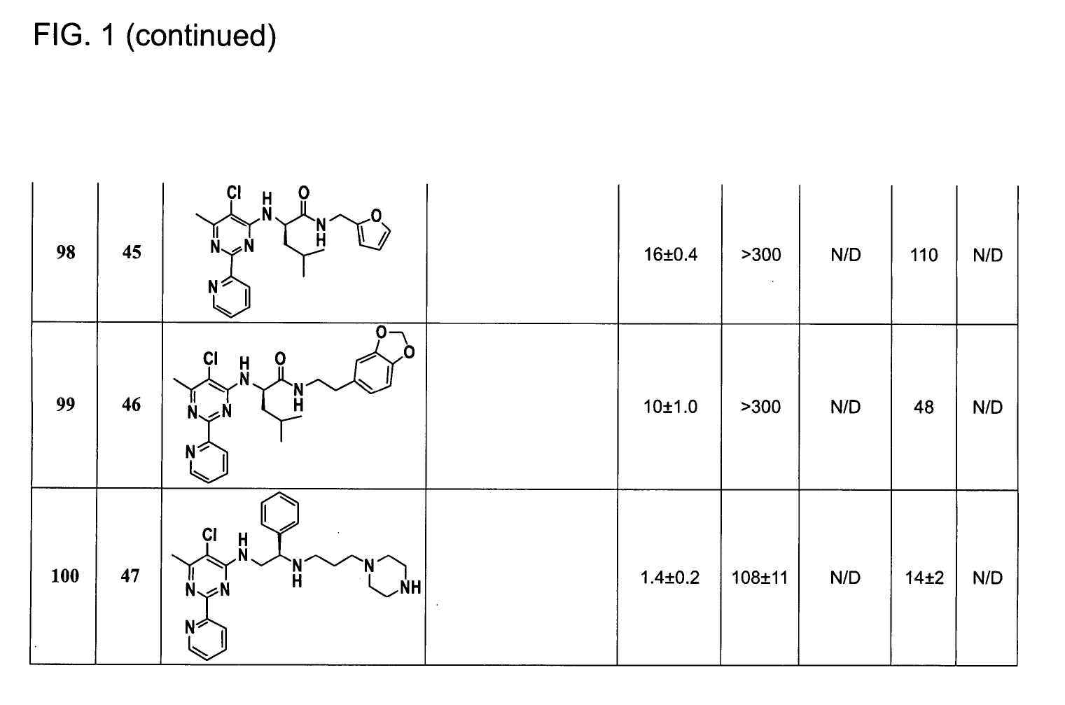

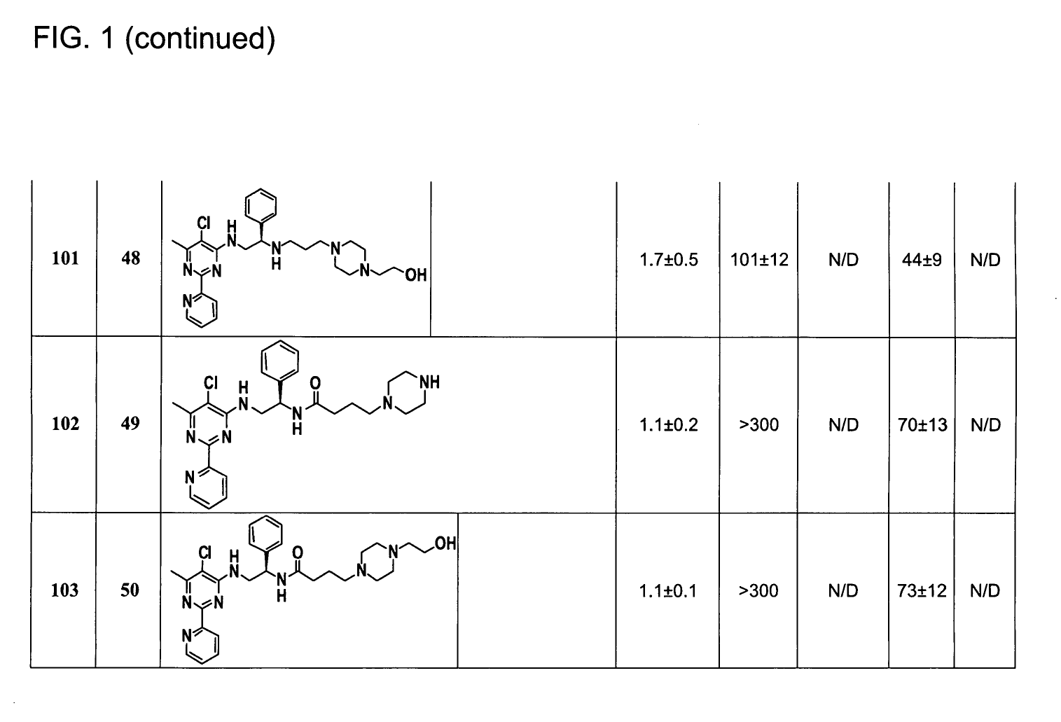

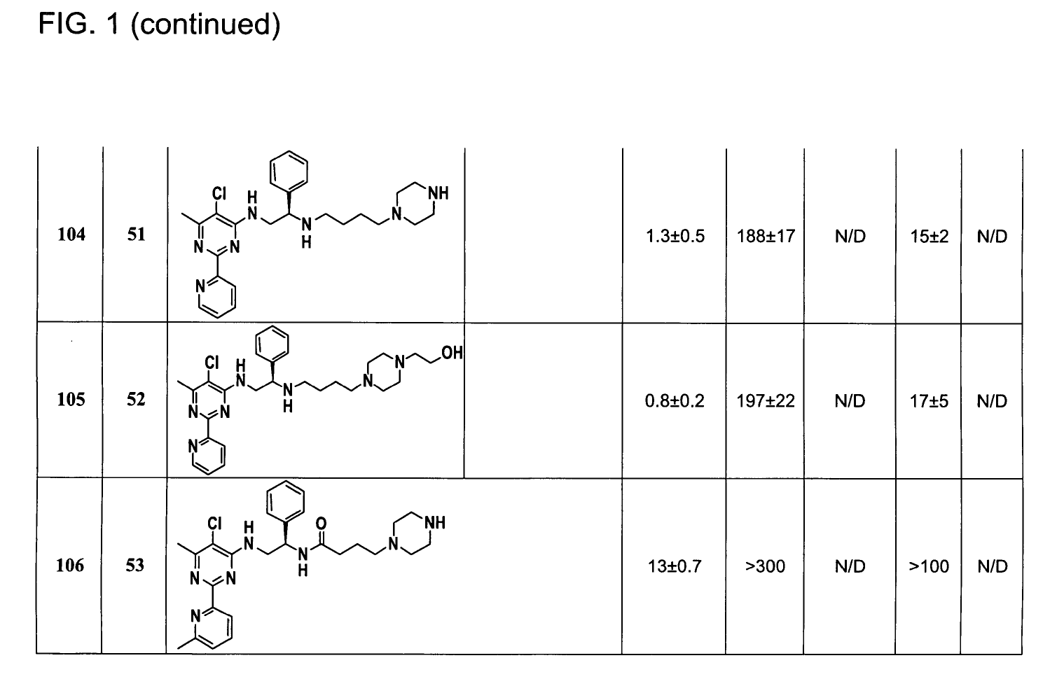

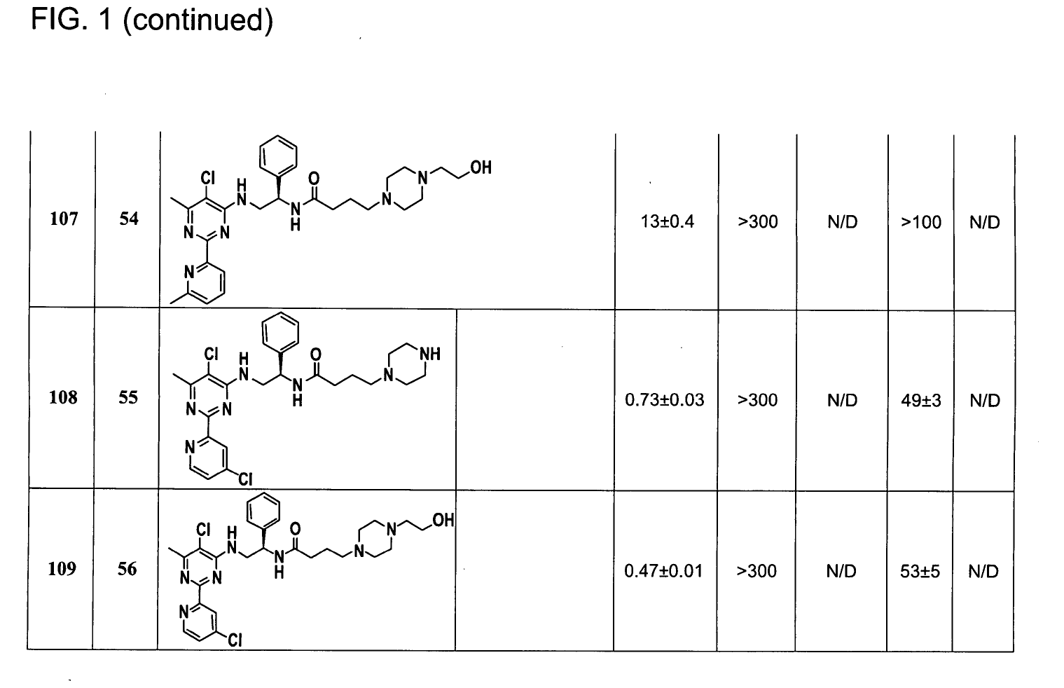

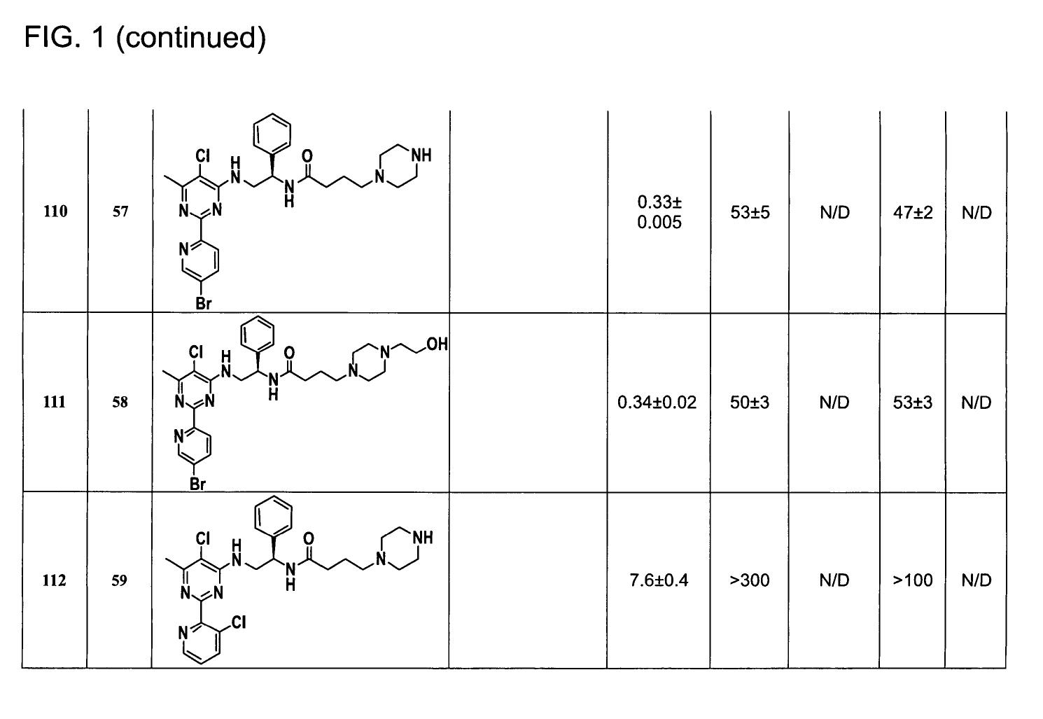

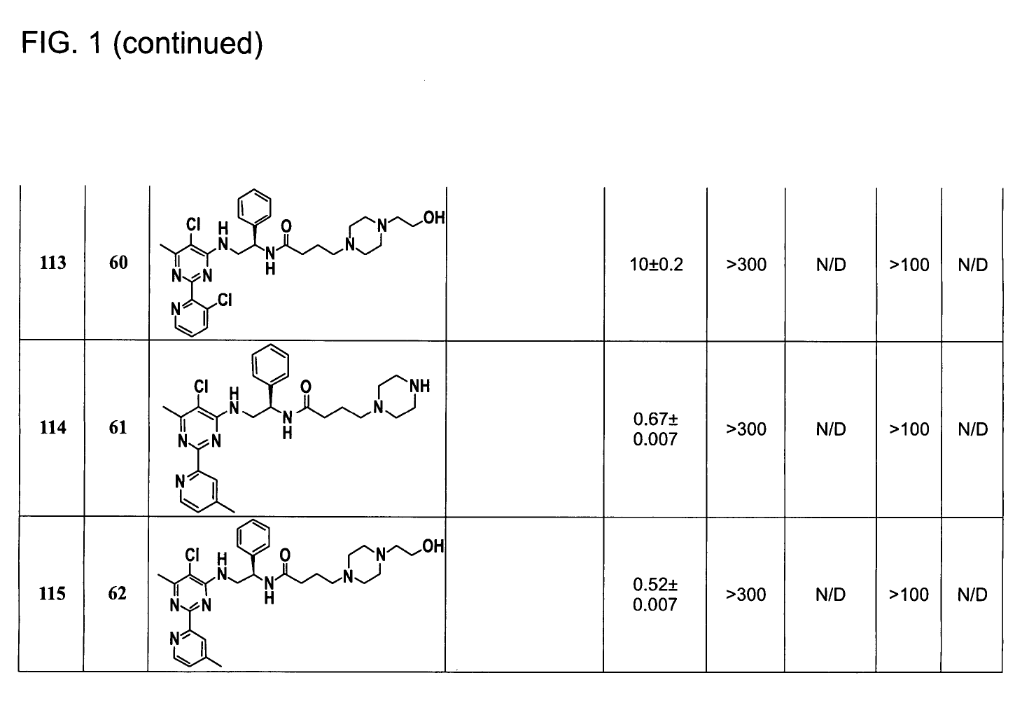

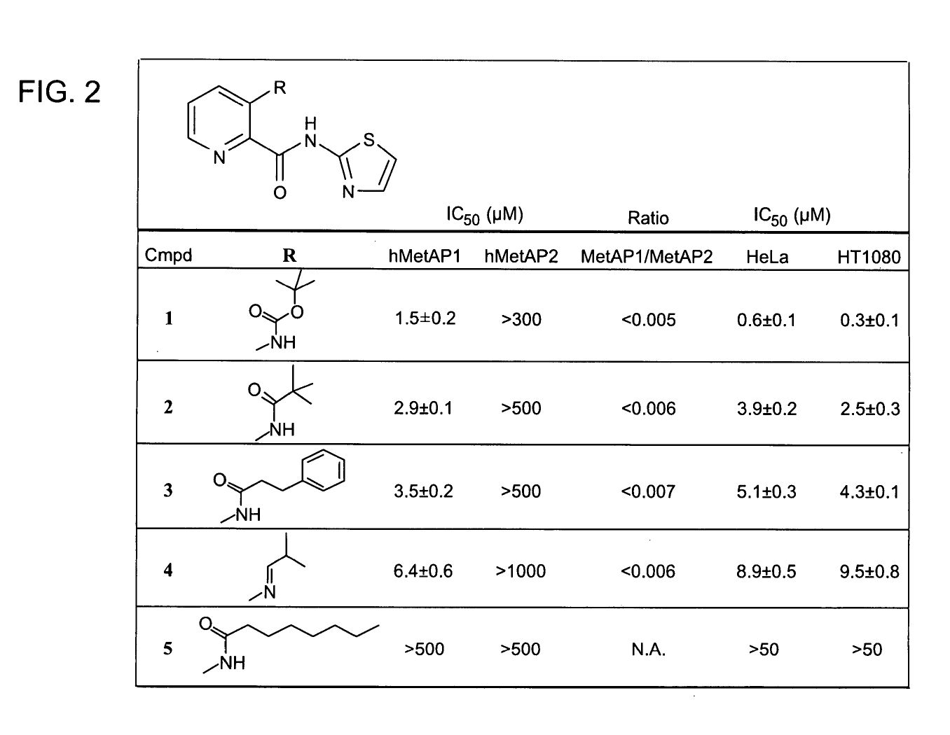

Various compounds were synthesized and tested against both human MetAP1 and MetAP2 in the presence of Cobalt (II) ions. As shown in FIGS. 1 and 2, numerous compounds were able to potently inhibit human MetAP1 enzymatic activity while none of them inhibited HsMetAP2 activity up to their solubility limits (300-1,000 μM). Various analogs were tested the presence of manganese ion as manganese (II) has been suggested as the physiological metal ion for HsMetAP2. HsMetAP2 remained unaffected by the highest concentrations of each compounds in the presence of manganese ion (data not shown). Taken together, these results suggested that the compounds of the invention are highly specific for HsMetAP1, rendering them useful molecular probes to elucidate the cellular function of HsMetAP1.

The effets of HsMetAP1 inhibitors on cell proliferation using a [³H]-thymidine incorporation assay was carried out (Hu, X., et al. (2006) Angew. Chem. Int. Ed Engl. 45, 3772-3775). Both HeLa and HT-1080 cells were inhibited with IC₅₀ values in the low micromolar range. There is a correlation between cellular inhibitory effects and HsMetAP1 inhibition.

### Compounds of the Invention Inhibit MetAP1 Inside Cells.

Although it was previously shown that pyridine-2-carboxylic acid derivatives selectively inhibit HsMetAP1 in vitro and block cell proliferation in culture, the causative relationship between these two effects remained to be established. As the first step to assess this relationship, a determination whether the compounds of the invention are capable of entering cells and inhibiting HsMetAP1 activity in vivo by examining the N-terminal initiator methionine status of a known protein substrate, 14-3-3γ (Towbin, H., et al. (2003) J. Biol. Chem. 278, 52964-52971). HeLa cells were incubated with various concentrations of various compounds of the invention for 24 h before they were harvested for Western Blot with a monoclonal antibody (clone HS23) specific for the methionylated N-terminal fragment of 14-3-3γ protein. Treatment with the compounds of the invention resulted in a dose-dependent increase in the amounts of N-terminal methionine-containing 14-3-3γ protein, compared with vehicle control, suggesting that the compounds of the inventio are capable of inhibiting HsMetAP1 activity inside cells.

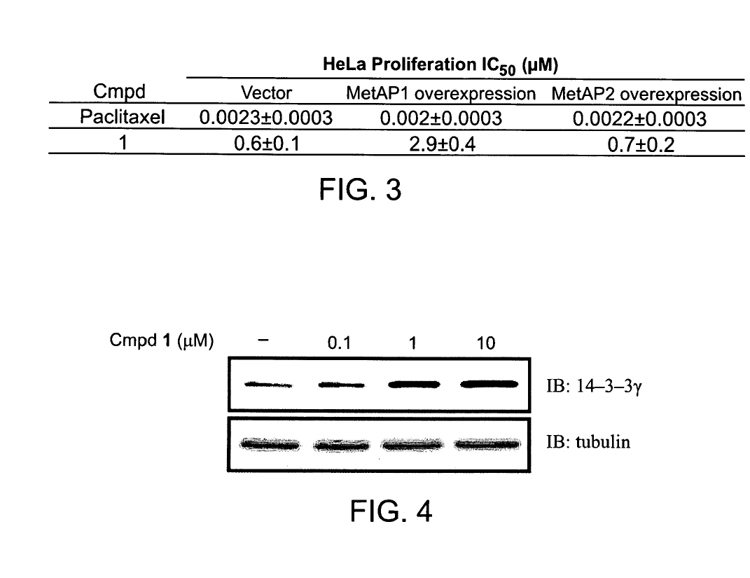

If the inhibition of cell proliferation by the compounds of the invention is due to the inhibition of HsMetAP1, it is expected that overexpression of HsMetAP1 should provide a gain of resistance to the inhibitors. HeLa cells were thus transfected with expression vectors for HsMetAP1, or HsMetAP2 as well as an empty vector as control. Overexpression of HsMetAPs was confirmed by Western blots. The growth of HeLa cells were not affected by different transfections. In certain cases, cells overexpressing HsMetAP1 (˜6-fold of control determined by Western blot) showed an approximately 5-fold decrease in the potency for the compounds of the invention in comparison to cells transfected with the vector (FIG. 3). In contrast, HeLa cells overexpressing HsMetAP2 remained as sensitive to various compounds of the invention as control cells, suggesting that HsMetAP1 plays a unique role in HeLa cell proliferation that could not be compensated for by HsMetAP2. When the same cells were treated with paclitaxel known to target tubulin, which is mechanistically unrelated to MetAP inhibitors, all three cell populations exhibited similar sensitivity, further supporting the notion that the compounds of the invention inhibit cell growth by inhibiting the cellular MetAP1 enzyme.

The Compounds of the Invention Inhibit Cell Growth by Delaying the Cell Cycle Progression through G2/M Phase

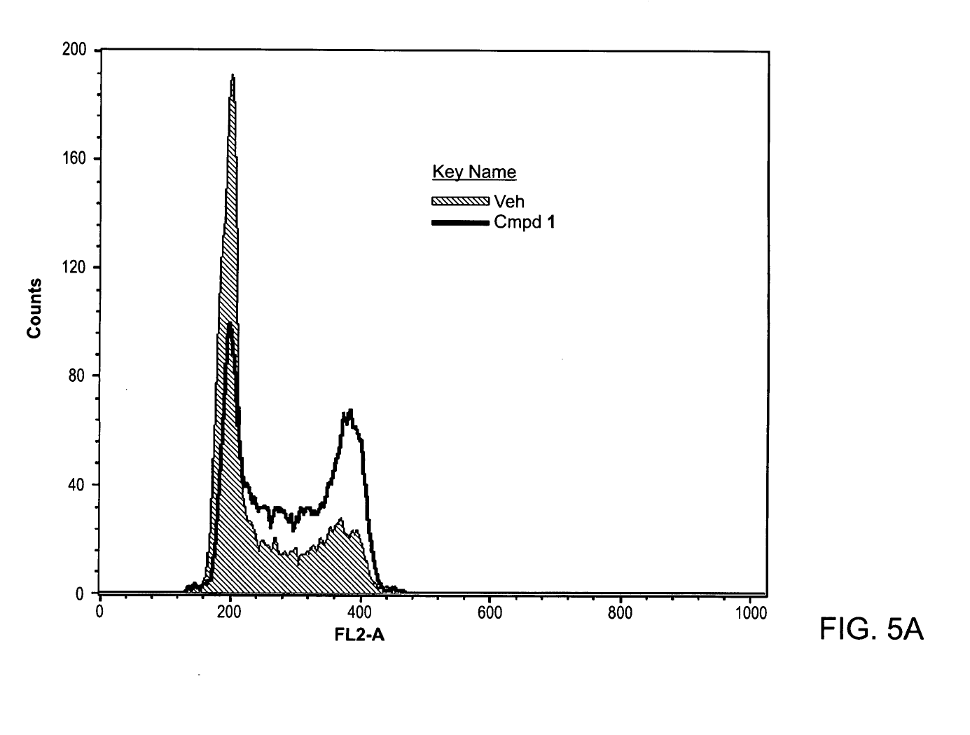

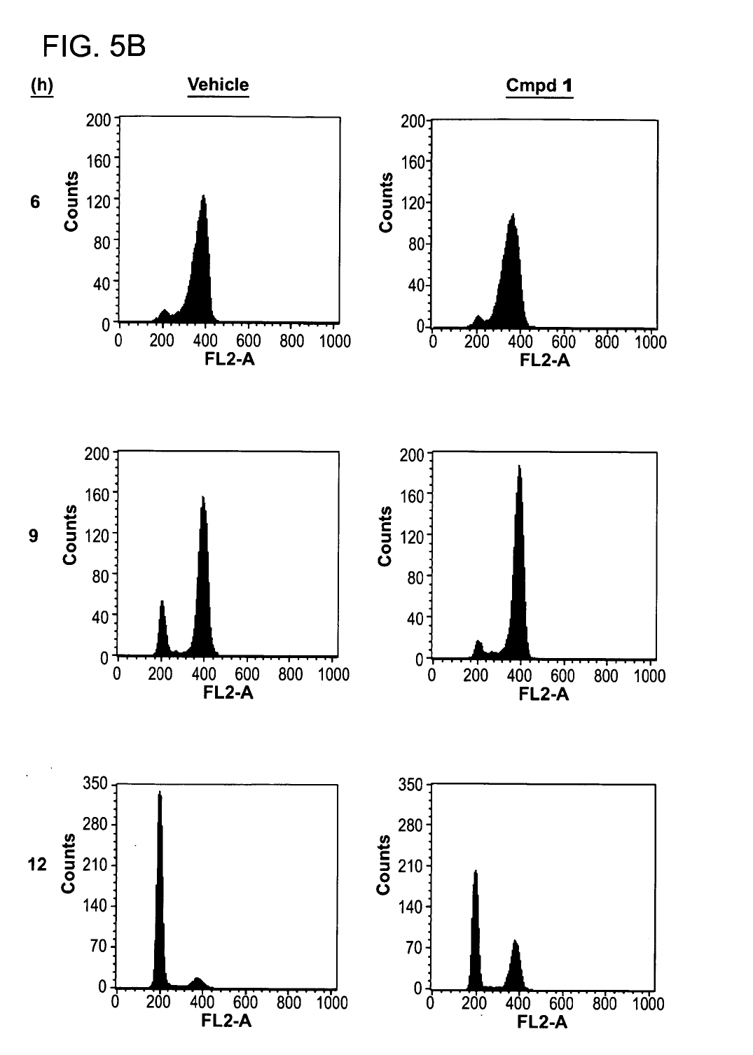

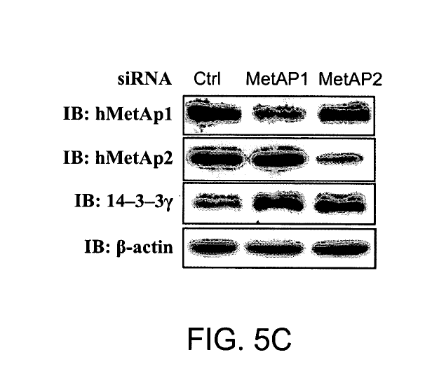

To understand the mechanism of cell proliferation inhibition by HsMetAP1 inhibitors, the effects on cell cycle progression were examined using flow cytometry (Kanzawa, T., et al. (2003) Br. J. Cancer 89, 922-929). Unsynchronized HeLa cells treated with vehicle control showed canonic distribution in G1, S and G2/M phases. However, treatment with various compounds of the invention led to a significant increase in cell populations at the G2/M phase (FIG. 5A). When HeLa cells were synchronized by double-thymidine at G1/S check point and released in the presence or absence of various compounds of the invention, respectively, no difference was observed at 6 h time point for these two populations of cells (FIG. 5B). However, 9- to 12-h after resumption of cell cycle, HeLa cells treated with various compounds of the invention exhibited a significant slower progression through G2/M phase, even though cells treated with various compounds of the invention eventually were able to complete mitosis at 16-hr post thymidine release (data not shown).

HsMetAP1 is Required for Timely Cell Cycle Progression through G2/M Phase

The G2/M delay caused by the active inhibitors 1 and 2 of FIG. 2 suggested that HsMetAP1 may be essential for timely progression through this phase of the cell cycle. To further verify this possibility, we took a complementary approach using selective siRNA duplexes to down-regulate the expression of HsMetAP1 and determined its consequence on cell cycle progression. HeLa cells were transfected with 100 nM siRNA duplexes for either HsMetAP1 or HsMetAP2 and the cellular protein levels were determined 48 h after transfection by Western blot analysis. As shown in FIG. 2C, HsMetAP1- and HsMetAP2-specific siRNA duplexes were able to down-regulate targeted proteins significantly in comparison with scrambled-duplex control without interfering with the expression of non-targeted MetAP or β-actin. In agreement with the downregulation of either HsMetAP1 or HsMetAP2 by their respective siRNAs, corresponding increases in methionylated 14-3-3γ proteins were observed, as expected (FIG. 5C).

We next used these siRNA duplexes to transfect HeLa cells 6 hrs before the initiation of double-thymidine synchronization of cell cycle. Cells were harvested at 0 h, 4 h, 8 h and 12 h-post second thymidine release, followed by cell cycle analysis with FACS. As shown in FIG. 5D, all cells were synchronized on G1/S checkpoint at 0 h and launched their genome replication at the 4-h time point with comparable speed. However, HsMetAP1 siRNA-treated cells showed significant delay in progression through G2/M phase at 8 h, in comparison with cells transfected with scrambled or HsMetAP2 siRNA duplexes (FIG. 5D). Eventually, all the cells were able to complete mitosis for a new cycle of cell proliferation at 12 h time point. Similar effects were observed in HT1080 cells during their G2/M phase progression (data not shown). Thus, HsMetAP1 is required for the precise progression through G2/M phase.

HsMetAP1-specific Inhibition Delayed Cyclin B Protein Degradation During Mitosis

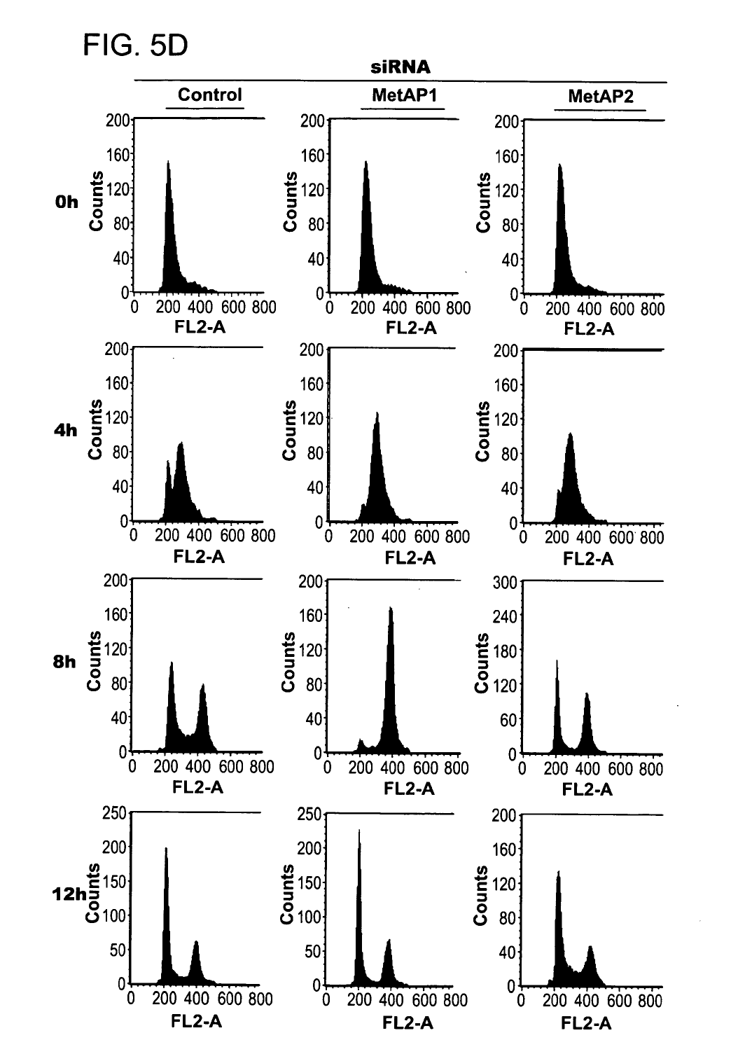

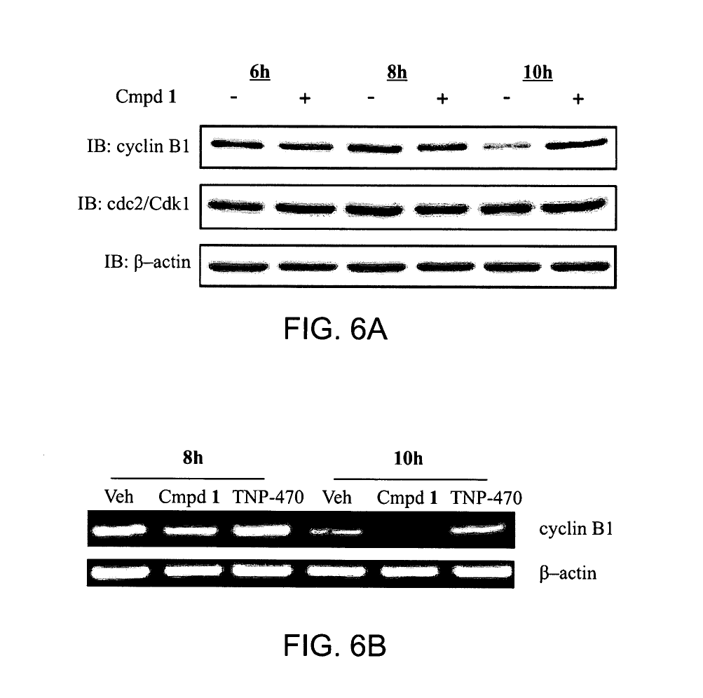

Proper cell cycle progression is regulated by different cyclin proteins and cyclin-dependent kinases (CDKs). Cdk1/cyclin B is the universal cell cycle regulator implicated in the G2/M phase transition. Exit from mitosis involves inactivation of CDK kinase through cyclin B degradation. To understand the molecular mechanism of HsMetAP1-specific inhibition, we examined the protein levels of cyclin B1 and cdc2/Cdk1 kinase during mitosis. When synchronized HeLa cells were released from thymidine arrest, cyclin B1 decreased dramatically between the 8-h and 10-h time points for vehicle control cells. However, cells treated with 1 of FIG. 2 still have significant amount of cyclin B1 protein at 10-h time point (FIG. 6A). Cyclin B1 protein level eventually dropped at the 12-h time point (data not shown). Interestingly, cdc2/Cdk1 protein level did not change under compound 1 treatment (FIG. 6A).

Cyclin B1 protein expression is regulated at both transcriptional and post-transcriptional levels. To dissect the regulatory mechanisms of cyclin B1 protein by HsMetAP1 inhibition, we determined the mRNA level of cyclin B1 by RT-PCR. As shown in FIG. 6B, cyclin B1 mRNA decreased during mitosis. Neither compound 1 of FIG. 2, the HsMetAP1-specific inhibitor, nor TNP-470, the HsMetAP2-specific inhibitor, altered the decrease of cyclin B1 mRNA level between 8 to 10 hours. These results indicated that delayed degradation of cyclin B1 protein is likely to be regulated at the post-transcriptional level.

### HsMetAP1-Specific Inhibition Induces Cellular Apoptosis

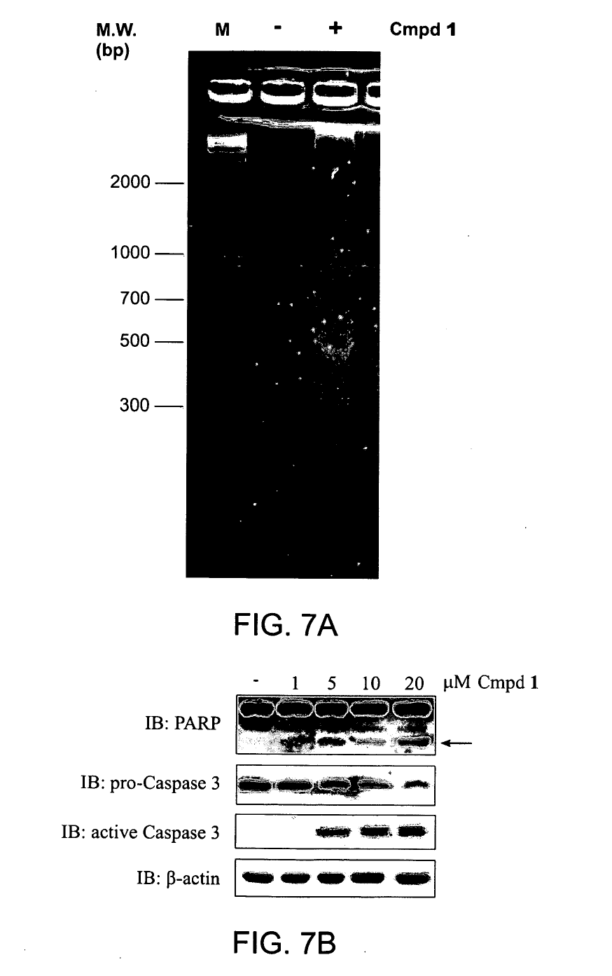

The G2/M phase transition is critical for proper cell division and a G2/M checkpoint disruption has been shown to cause apoptosis in a number of tumor cell lines (Rieder, C. L. & Maiato, H. (2004) Dev. Cell 7, 637-651). To test if 1-specific inhibition could cause apoptosis, we determined their effect on JurKat T cells. As shown in FIG. 7A, treatment of Jurkat T cells with compound 1 of FIG. 2 resulted in fragmentation of nucleosomal DNA, judged by DNA ladder pattern, as the hallmark of apoptosis. In addition to DNA laddering, we also examined the proteolytic cleavages of poly (ADP-ribose) polymerase (PARP) and pro-caspase-3. Treatment with compound 1 of FIG. 2 for 24 hrs resulted in a dosage-dependent cleavage of PARP protein. Concurrently, the full-length pro-caspase 3 decreased in a similar dose-dependent fashion, complemented by the appearance of the active fragment of caspase-3 (FIG. 7B). Similar results have been observed in a B-cell non-Hodgkin's lymphoma cell line (Karpas 1106) (data not shown). Thus, by causing a delay in G2/M phase, inhibitors of HsMetAP1 are capable of inducing apoptosis among cancer cells, suggesting that HsMetAP1 may be a useful target for anticancer agents.

Small molecules have played an important part in the elucidation of functions of genes. Early examples include the use of the immunosuppressant drugs CyclosporinA and FK506 to reveal the important role of the protein phosphatase calcineurin mediating intracellular calcium signaling pathway. The application of another immunosuppressive natural product, rapamycin, has shed significant light on the TOR kinases in myriad signaling processes from cytokine signaling to nutrient sensing. Similarly, the identification of MetAP2 as the cellular target for the fumagillin family of anti-angiogenic natural products has helped to reveal a unique function of this otherwise universally expressed general-processing enzyme in endothelial and T cells. In contrast to MetAP2, study of the cellular functions of MetAP1, however, has been hampered in part by the lack of an inhibitor with sufficient selectivity.

Since the identification of HsMetAP2 as a target of fumagillin and ovalicin, the MetAP family of enzymes has been under increasing scrutiny as targets for developing antibacterial, antifungal and anticancer drugs. Among the MetAP inhibitors reported, the pyridine-2-carboxylic acid thiazole-2-ylamide class has emerged as potent inhibitors of both E. coli and yeast MetAP1. Moreover, members of this class of compounds have been subsequently shown to inhibit recombinant HsMetAP I (Li, J. Y., et al. (2004) Biochemistry 43, 7892-7898; Cui, Y. M., et al. (2005) Bioorg. Med. Chem. Lett. 15, 4130-4135). It remained unknown, however, whether this family of MetAP1 inhibitors also cross-interact with MetAP2. We synthesized several analogs of this class of inhibitors and determined their specificity for the two isoforms of HsMetAPs. Gratifyingly, various compounds synthesized (FIG. 2) exhibited exquisite specificity for HsMetAP1, with ratios in IC₅₀ values for the two enzymes over 200 fold, rendering these inhibitors useful probes for the cellular function of HsMetAP1 without the complication of cross inhibition on HsMetAP2 at relatively low concentrations.

The effects of HsMetAP1 inhibitors on G2/M phase transition appear to be significant, yet different from those seen with other cytotoxic anticancer drugs such as paclitaxel or colchicine that also inhibit cell cycle at the G2/M phase. Rather than a sustained blockade of cells through the G2/M phase, compound 1 of FIG. 2 and its analogs caused a 3-4 h delay of cell cycle progression through the G2/M phase and allowed cells to eventually reach G1 phase to resume another round of cell cycle. Although this delay, not blockade, in G2/M phase by HsMetAP1 inhibitors is plausibly overcome by some cancer cell lines, it led to apoptosis of both Jurkat and Karpas 1106 lymphoma cell lines. It is possible that in both Jurkat and Karpas 1106 lymphoma cell lines, the delay in G2/M phase progression by HsMetAP1 inhibitors is not compatible with the preexisting disruption of the G2/M checkpoint control and causes cells to undergo apoptosis. As such, HsMetAP1 inhibitors represent a novel mechanistic class of G2/M phase inhibitors.

Timely degradation of cyclin B protein is critical for exit from mitosis during cell cycle progression. Our results suggested that inhibition of HsMetAP1 regulates cyclin B protein level through a post-transcriptional mechanism. The penultimate residue for cyclin B protein is alanine, which qualifies cyclin B as a substrate for MetAP; however, it remains unclear whether N-terminal methionine retention would directly account for the delayed degradation of cyclin B protein. It is equally reasonable that the delayed-degradation effect on cyclin B protein is indirect, as a consequence of the N-terminal methionine retention of another protein. Our further experiments have demonstrated that the compounds of the invention are capable of promoting cellular apoptosis by the activation of caspase-3 and cleavage of PARP protein. It is thus believed that inhibition of HsMetAP1 slows down cell cycle progression, activates G2/M checkpoint and eventually leads to apoptosis for cell proliferation inhibition.

The high-resolution crystal structures of the complexes between HsMetAP1 and compounds 1 and 2 of FIG. 2 threw significant new light on the highly-specific molecular interaction between the enzyme and the inhibitors. Compounds 1 and 2 of FIG. 2 differ by only one oxygen atom. The orientation of the tert-butyl group is different in both the compounds with respect to the rest of the molecule. In 2, the tert-butyl group points in a direction close to the scaffold, which we refer it as a syn conformation, while that in 1, it points away in a trans conformation (data not presented). This directionality of the tert-butyl groups seems to play an important role in the selectivity as suggested by the biochemical data, both from our present study and from the results of Luo et al ((2003) J. Med. Chem. 46, 2631-2640). Structural data suggest that the overall direction of the tert-butyl group is away from the protein surface in the ester-based side chain of 1 of FIG. 2 whereas it points into the hydrophobic depression on the protein surface in the ketone-based compound 2 of FIG. 2. Compound 3 of FIG. 2 also follows the similar trend as 2 although it has slightly lower affinity. It is possible that the aromatic side chain of compound 3 forms π-π stacking interactions with either or both aromatic rings of Tyr195 and Tyr196. The double bond in the side chain of 4 of FIG. 2 imposes extra rigidity, limits the freedom of orientation that is necessary for interaction with protein side chains, thus explaining the lower affinity. In addition, it is also possible the two methoxy groups in 4 may play a role in the lowering of affinity. The compounds described here have 100-1000 fold lower affinity towards the type 2 human enzyme compared to that with type 1 enzyme (FIG. 2). The side chain of compound 2, which is close to the surface of the protein in the HsMetAP1 complex, seems to have a more severe steric clash with the side chain of Tyr444 of the HsMetAP2 compared to that of the compound 1. Such steric interactions provide the clue to the difference in affinity of 1 and 2 towards HsMetAP2.A similar observation was made for pyridinylpyrimidine compounds described in our previous study (Hu, X., et al. (2006) Angew. Chem. Int. Ed Engl. 45, 3772-3775). Our in vitro enzymatic assay suggested that the compounds of the invention are selective inhibitors.

### V. Pharmaceutical Compositions/Methods of Administration

In one aspect, the invention provides a composition comprising a compound of the invention and an additional therapeutic agent. In one embodiment, the additional therapeutic agent is a methionine aminopeptidase-inhibiting compound. In another embodiment, the additional therapeutic agent is an anticancer compound.

In another aspect, the invention provides a pharmaceutical composition comprising a compound of the invention and a pharmaceutically suitable excipient.

The present invention is also directed to pharmaceutical compositions comprising an effective amount of one or more compounds according to the present invention (including a pharmaceutically acceptable salt, thereof), optionally in combination with a pharmaceutically acceptable carrier, excipient or additive.

A “pharmaceutically acceptable derivative or prodrug” means any pharmaceutically acceptable salt, ester, salt of an ester, or other derivative of a compound of this invention which, upon administration to a recipient, is capable of providing (directly or indirectly) a compound of this invention.

The compounds of the present invention may be administered orally, parenterally, by inhalation spray, rectally, vaginally, or topically in dosage unit formulations containing conventional pharmaceutically acceptable carriers, adjuvants, and vehicles. The term parenteral as used herein includes, subcutaneous, intravenous, intramuscular, intrasternal, infusion techniques, intraperitoneally, eye or ocular, intrabuccal, transdermal, intranasal, into the brain, including intracranial and intradural, into the joints, including ankles, knees, hips, shoulders, elbows, wrists, directly into tumors, and the like, and in suppository form.

The pharmaceutically active compounds of this invention can be processed in accordance with conventional methods of pharmacy to produce medicinal agents for administration to patients, including humans and other mammals.

Modifications of the active compound can affect the solubility, bioavailability and rate of metabolism of the active species, thus providing control over the delivery of the active species. Further, the modifications can affect the anti-angiogenesis activity of the compound, in some cases increasing the activity over the parent compound. This can easily be assessed by preparing the derivative and testing its activity according to known methods well within the routineer's skill in the art.

Pharmaceutical compositions based upon these chemical compounds comprise the above-described compounds in a therapeutically effective amount for treating diseases and conditions which have been described herein, optionally in combination with a pharmaceutically acceptable additive, carrier and/or excipient. One of ordinary skill in the art will recognize that a therapeutically effective amount of one of more compounds according to the present invention will vary with the infection or condition to be treated, its severity, the treatment regimen to be employed, the pharmacokinetics of the agent used, as well as the patient (animal or human) treated.

To prepare the pharmaceutical compositions according to the present invention, a therapeutically effective amount of one or more of the compounds according to the present invention is preferably intimately admixed with a pharmaceutically acceptable carrier according to conventional pharmaceutical compounding techniques to produce a dose. A carrier may take a wide variety of forms depending on the form of preparation desired for administration, e.g., oral, topical or parenteral, including gels, creams ointments, lotions and time released implantable preparations, among numerous others. In preparing pharmaceutical compositions in oral dosage form, any of the usual pharmaceutical media may be used.

The active compound is included in the pharmaceutically acceptable carrier or diluent in an amount sufficient to deliver to a patient a therapeutically effective amount for the desired indication, without causing serious toxic effects in the patient treated.

Oral compositions will generally include an inert diluent or an edible carrier. They may be enclosed in gelatin capsules or compressed into tablets. For the purpose of oral therapeutic administration, the active compound or its prodrug derivative can be incorporated with excipients and used in the form of tablets, troches, or capsules. Pharmaceutically compatible binding agents, and/or adjuvant materials can be included as part of the composition.

The tablets, pills, capsules, troches and the like can contain any of the following ingredients, or compounds of a similar nature: a binder such as microcrystalline cellulose, gum tragacanth or gelatin; an excipient such as starch or lactose, a dispersing agent such as alginic acid or corn starch; a lubricant such as magnesium stearate; a glidant such as colloidal silicon dioxide; a sweetening agent such as sucrose or saccharin; or a flavoring agent such as peppermint, methyl salicylate, or orange flavoring. When the dosage unit form is a capsule, it can contain, in addition to material-of the above type, a liquid carrier such as a fatty oil. In addition, dosage unit forms can contain various other materials which modify the physical form of the dosage unit, for example, coatings of sugar, shellac, or enteric agents.

Formulations of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil emulsion and as a bolus, etc.

A tablet may be made by compression or molding, optionally with one or more accessory ingredients. Compressed tablets may be prepared by compressing in a suitable machine the active ingredient in a free-flowing form such as a powder or granules, optionally mixed with a binder, lubricant, inert diluent, preservative, surface-active or dispersing agent. Molded tablets may be made by molding in a suitable machine a mixture of the powdered compound moistened with an inert liquid diluent. The tablets optionally may be coated or scored and may be formulated so as to provide slow or controlled release of the active ingredient therein.

Methods of formulating such slow or controlled release compositions of pharmaceutically active ingredients, are known in the art and described in several issued US Patents, some of which include, but are not limited to, U.S. Pat. Nos. 3,870,790; 4,226,859; 4,369,172; 4,842,866 and 5,705,190, the disclosures of which are incorporated herein by reference in their entireties. Coatings can be used for delivery of compounds to the intestine (see, e.g., U.S. Pat. Nos. 6,638,534, 5,541,171, 5,217,720, and 6,569,457, and references cited therein).

The active compound or pharmaceutically acceptable salt thereof may also be administered as a component of an elixir, suspension, syrup, wafer, chewing gum or the like. A syrup may contain, in addition to the active compounds, sucrose or fructose as a sweetening agent and certain preservatives, dyes and colorings and flavors.

Solutions or suspensions used for parenteral, intradermal, subcutaneous, or topical application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art.

A skilled artisan will recognize that in addition to tablets, other dosage forms can be formulated to provide slow or controlled release of the active ingredient. Such dosage forms include, but are not limited to, capsules, granulations and gel-caps.

Liposomal suspensions may also be pharmaceutically acceptable carriers. These may be prepared according to methods known to those skilled in the art. For example, liposomal formulations may be prepared by dissolving appropriate lipid(s) in an inorganic solvent that is then evaporated, leaving behind a thin film of dried lipid on the surface of the container. An aqueous solution of the active compound are then introduced into the container. The container is then swirled by hand to free lipid material from the sides of the container and to disperse lipid aggregates, thereby forming the liposomal suspension. Other methods of preparation well known by those of ordinary skill may also be used in this aspect of the present invention.

The formulations may conveniently be presented in unit dosage form and may be prepared by conventional pharmaceutical techniques. Such techniques include the step of bringing into association the active ingredient and the pharmaceutical carrier(s) or excipient(s). In general, the formulations are prepared by uniformly and intimately bringing into association the active ingredient with liquid carriers or finely divided solid carriers or both, and then, if necessary, shaping the product.

Formulations and compositions suitable for topical administration in the mouth include lozenges comprising the ingredients in a flavored basis, usually sucrose and acacia or tragacanth; pastilles comprising the active ingredient in an inert basis such as gelatin and glycerin, or sucrose and acacia; and mouthwashes comprising the ingredient to be administered in a suitable liquid carrier.

Formulations suitable for topical administration to the skin may be presented as ointments, creams, gels and pastes comprising the ingredient to be administered in a pharmaceutical acceptable carrier. A preferred topical delivery system is a transdermal patch containing the ingredient to be administered.

Formulations for rectal administration may be presented as a suppository with a suitable base comprising, for example, cocoa butter or a salicylate.

Formulations suitable for nasal administration, wherein the carrier is a solid, include a coarse powder having a particle size, for example, in the range of 20 to 500 microns which is administered in the manner in which snuff is administered, i.e., by rapid inhalation through the nasal passage from a container of the powder held close up to the nose. Suitable formulations, wherein the carrier is a liquid, for administration, as for example, a nasal spray or as nasal drops, include aqueous or oily solutions of the active ingredient.

Formulations suitable for vaginal administration may be presented as pessaries, tampons, creams, gels, pastes, foams or spray formulations containing in addition to the active ingredient such carriers as are known in the art to be appropriate.

The parenteral preparation can be enclosed in ampoules, disposable syringes or multiple dose vials made of glass or plastic. If administered intravenously, preferred carriers include, for example, physiological saline or phosphate buffered saline (PBS).

For parenteral formulations, the carrier will usually comprise sterile water or aqueous sodium chloride solution, though other ingredients including those which aid dispersion may be included. Of course, where sterile water is to be used and maintained as sterile, the compositions and carriers must also be sterilized. Injectable suspensions may also be prepared, in which case appropriate liquid carriers, suspending agents and the like may be employed.

Formulations suitable for parenteral administration include aqueous and non-aqueous sterile injection solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets of the kind previously described.

Administration of the active compound may range from continuous (intravenous drip) to several oral administrations per day (for example, Q.I.D.) and may include oral, topical, eye or ocular, parenteral, intramuscular, intravenous, sub-cutaneous, transdermal (which may include a penetration enhancement agent), buccal and suppository administration, among other routes of administration, including through an eye or ocular route.

Application of the subject therapeutics may be local, so as to be administered at the site of interest. Various techniques can be used for providing the subject compositions at the site of interest, such as injection, use of catheters, trocars, projectiles, pluronic gel, stents, sustained drug release polymers or other device which provides for internal access. Where an organ or tissue is accessible because of removal from the patient, such organ or tissue may be bathed in a medium containing the subject compositions, the subject compositions may be painted onto the organ, or may be applied in any convenient way.

The compound may be administered through a device suitable for the controlled and sustained release of a composition effective in obtaining a desired local or systemic physiological or pharmacological effect. The method includes positioning the sustained released drug delivery system at an area wherein release of the agent is desired and allowing the agent to pass through the device to the desired area of treatment.

It should be understood that in addition to the ingredients particularly mentioned above, the formulations of the present invention may include other agents conventional in the art having regard to the type of formulation in question, for example, those suitable for oral administration may include flavoring agents.

In certain pharmaceceutical dosage forms, the pro-drug form of the compounds may be preferred. One of ordinary skill in the art will recognize how to readily modify the present compounds to pro-drug forms to facilitate delivery of active compounds to a targeted site within the host organism or patient. The routineer also will take advantage of favorable pharmacokinetic parameters of the pro-drug forms, where applicable, in delivering the present compounds to a targeted site within the host organism or patient to maximize the intended effect of the compound.

Preferred prodrugs include derivatives where a group which enhances aqueous solubility or active transport through the gut membrane is appended to the structure of formulae described herein. See, e.g., Alexander, J. et al. Journal of Medicinal Chemistry 1988, 31, 318-322; Bundgaard, H. Design of Prodrugs; Elsevier: Amsterdam, 1985; pp 1-92; Bundgaard, H.; Nielsen, N. M. Journal of Medicinal Chemistry 1987, 30, 451-454; Bundgaard, H. A Textbook of Drug Design and Development; Harwood Academic Publ.: Switzerland, 1991; pp 113-191; Digenis, G. A. et al. Handbook of Experimental Pharmacology 1975, 28, 86-112; Friis, G. J.; Bundgaard, H. A Textbook of Drug Design and Development; 2 ed.; Overseas Publ.: Amsterdam, 1996; pp 351-385; Pitman, I. H. Medicinal Research Reviews 1981, 1, 189-214. The prodrug forms may be active themselves, or may be those such that when metabolized after administration provide the active therapeutic agent in vivo.

Pharmaceutically acceptable salt forms may be the preferred chemical form of compounds according to the present invention for inclusion in pharmaceutical compositions according to the present invention.

Certain of the compounds, in pharmaceutical dosage form, may be used as agents for preventing a disease or condition from manifesting itself. In certain pharmaceutical dosage forms, the pro-drug form of the compounds according to the present invention may be preferred. In particular, prodrug forms which rely on C₁ to C₂₀ ester groups or amide groups (preferably a hydroxyl, free amine or substituted nitrogen group) which may be transformed into, for example, an amide or other group may be particularly useful in this context.

The present compounds or their derivatives, including prodrug forms of these agents, can be provided in the form of pharmaceutically acceptable salts. As used herein, the term pharmaceutically acceptable salts or complexes refers to appropriate salts or complexes of the active compounds according to the present invention which retain the desired biological activity of the parent compound and exhibit limited toxicological effects to normal cells. Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, and polyglutamic acid, among others; (b) base addition salts formed with metal cations such as zinc, calcium, sodium, potassium, and the like, among numerous others.

Preferred unit dosage formulations are those containing a daily dose or unit, daily sub-dose, as hereinabove recited, or an appropriate fraction thereof, of the administered ingredient.

The dosage regimen for treating a disorder or a disease with the hMetAP-inhibiting compounds of this invention and/or compositions of this invention is based on a variety of factors, including the type of disease, the age, weight, sex, medical condition of the patient, the severity of the condition, the route of administration, and the particular compound employed. Thus, the dosage regimen may vary widely, but can be determined routinely using standard methods.

The amounts and dosage regimens administered to a subject will depend on a number of factors, such as the mode of administration, the nature of the condition being treated, the body weight of the subject being treated and the judgment of the prescribing physician.

The amount of compound included within therapeutically active formulations according to the present invention is an effective amount for treating the infection or condition. In general, a therapeutically effective amount of the present preferred compound in dosage form usually ranges from slightly less than about 0.025 mg/kg/day to about 2.5 g/kg/day, preferably about 0.1 mg/kg/day to about 100 mg/kg/day of the patient or considerably more, depending upon the compound used, the condition or infection treated and the route of administration, although exceptions to this dosage range may be contemplated by the present invention. In its most preferred form, compounds according to the present invention are administered in amounts ranging from about 1 mg/kg/day to about 100 mg/kg/day. The dosage of the compound will depend on the condition being treated, the particular compound, and other clinical factors such as weight and condition of the patient and the route of administration of the compound. It is to be understood that the present invention has application for both human and veterinary use.

The compound is conveniently administered in any suitable unit dosage form, including but not limited to one containing 1 to 3000 mg, preferably 5 to 500 mg of active ingredient per unit dosage form. An oral dosage of 10-250 mg is usually convenient.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

In certain embodiments, the compound is administered once daily; in other embodiments, the compound is administered twice daily; in yet other embodiments, the compound is administered once every two days, once every three days, once every four days, once every five days, once every six days, once every seven days, once every two weeks, once every three weeks, once every four weeks, once every two months, once every six months, or once per year. The dosing interval can be adjusted according to the needs of individual patients. For longer intervals of administration, extended release or depot formulations can be used.

The compounds of the invention can be used to treat diseases and disease conditions that are acute, and may also be used for treatment of chronic conditions. In certain embodiments, the compounds of the invention are administered for time periods exceeding two weeks, three weeks, one month, two months, three months, four months, five months, six months, one year, two years, three years, four years, or five years, ten years, or fifteen years; or for example, any time period range in days, months or years in which the low end of the range is any time period between 14 days and 15 years and the upper end of the range is between 15 days and 20 years (e.g., 4 weeks and 15 years, 6 months and 20 years). In some cases, it may be advantageous for the compounds of the invention to be administered for the remainder of the patient's life. In preferred embodiments, the patient is monitored to check the progression of the disease or disorder, and the dose is adjusted accordingly. In preferred embodiments, treatment according to the invention is effective for at least two weeks, three weeks, one month, two months, three months, four months, five months, six months, one year, two years, three years, four years, or five years, ten years, fifteen years, twenty years, or for the remainder of the subject's life.

In one aspect, the invention provides a pharmaceutical composition comprising a compound of formula I and a pharmaceutically suitable excipient.

In another aspect, the invention provides a kit comprising an effective amount of a compound of formula I in unit dosage form, together with instructions for administering the compound to a subject suffering from or susceptible to a hMetAP1 related disease.

Still other objects, features, and attendant advantages of the present invention will become apparent to those skilled in the art from a reading of the preceding detailed description of embodiments constructed in accordance therewith, taken in conjunction with any accompanying drawings.

The invention will be further described in the following examples. It should be understood that these examples are for illustrative purposes only and are not to be construed as limiting this invention in any manner.

### EXAMPLES

GENERAL METHODS. ¹H- and ¹³C-NMR spectra were recorded at 400 MHz. Solvents used for extraction and purification were HPLC grade from either Fisher or Aldrich. Anhydrous tetrahydrofuran (THF) was distilled from sodium/benzophenone under dry argon. Dichloromethane (CH₂Cl₂) was distilled from calcium hydride under argon. Other anhydrous solvents and reagents were from Aldrich. Thin layer chromatography (TLC) was run on EM Science 250-μm analytical plates coated with silica gel GF₂₅₄. Column chromatography was performed using Aldrich silical gel, 200-400 mesh, 60 Å. Solvent mixtures are in volume/volume ratio.

### Example 1

### Synthesis of 5-chloro-6-methyl-4-(2-phenylethanethio)-2-(pyridin-2-yl)pyrimidine (2)

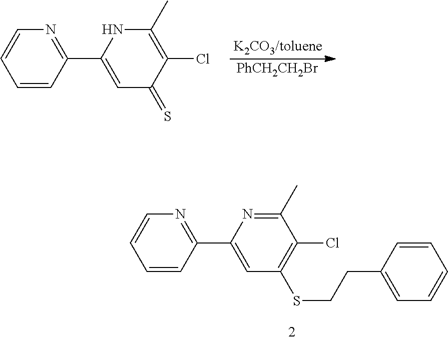

Anhydrous potassium carbonate (415 mg, 3 mmol) was added to a solution of 5-Chloro-6-methyl-2-(pyridin-2-yl)pyrimidin-4-thione (238 mg, 1 mmol) in toluene (15 mL) and the suspension was stirred at 60° C. for 20 min. Phenethyl bromide (222 mg, 1.2 mmol) was added to the reaction mixture and the stirring was continued for an additional 8 h. The reaction mixture was cooled to rt, quenched with water (25 mL) and the mixture was extracted with EtOAc (2×20 mL). The combined organic layer was concentrated and the crude product was purified by flash column chromatography over silica gel (eluent: EtOH/CHCl₃=5:95) to afford pyrimidine as an off-white solid (314 mg, 92%).

MALDI (matrix: DHB): m/z 343 (45%, M⁺+1), 365 (66%, M+Na⁺). ¹H NMR (400 MHz, acetone-d₆): δ 2.71 (s, 3H), 2.91 (t, J=7.6 Hz, 2H), 3.45 (t, J=7.6 Hz, 2H), 7.15 (m, 5H), 7.37 (app. dd, J=8.2 & 5.6 Hz, 1H), 7.84 (app t, J=8.2 Hz, 1H), 8.1 (d, J=8.2 Hz, 1H), 8.75 (d, J=5.6 Hz, 1H).

### Example 2

### Synthesis of 4, 5, 6 and 7

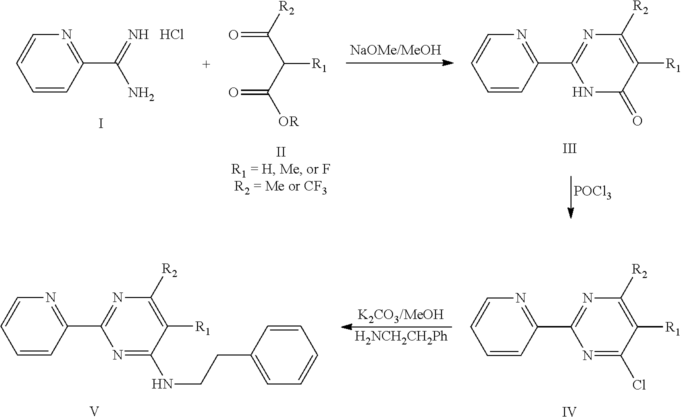

Tetrasubstituted pyrimidines V were synthesized following the procedure of Medwid, J. B. et al. (J. Org. Chem. 1990, 33, 1230-41.) The pure products were obtained after flash column chromatography over silica gel (eluent: 5-10% EtOH in CH₂Cl₂).

### Pyrimidine 4 (4: R¹═H, R²=Me):

MALDI (matrix: DHB): m/z 291 (100%, M⁺+1). ¹H NMR (400 MHz, acetone-d₆): δ 2.32 (s, 3H), 2.93 (t, J=7.6 Hz, 2H), 3.65 (m, 2H), 6.92 (t, J=7.8 Hz, 2H), 7.24 (t, J=8.1 Hz, 3H), 7.58 (d, J=7.8 Hz, 3H), 7.66 (ddd, J=8.2, 5.6, & 1.9 Hz, 1H), 7.86 (dd, J=8.2 & 1.9 Hz, 1H), 8.08 (dd, J=5.6 & 1.9 Hz, 1H), 8.13 (br s, 1H), 8.76 (d, J=5.6 Hz, 1H).

### Pyrimidine 5 (5: R¹=Me, R²═CF₃):

MALDI (matrix: DHB): m/z 360 (100%, M⁺+2), 381 (96%, M+Na⁺), 397 (95%, M+K⁺). ¹H NMR (400 MHz, acetone-d₆): δ 2.23 (s, 3H), 3.03 (t, J=6.7 Hz, 2H), 3.85 (t, J=6.7 Hz, 2H), 6.95 (br s, 1H), 7.37 (m, 5H), 7.44 (ddd, J=8.2, 2.4, & 1.8 Hz, 1H), 7.96 (ddd, J=8.2, 2.4, & 1.8 Hz, 1H), 8.42 (d, J=8.2 Hz, 1H), 8.68 (dd, J=2.4 & 1.8 Hz, 1H).

### Pyrimidine 6 (6: R¹═F, R²=Me):

MALDI (matrix: DHB): m/z 309 (100%, M⁺+1), 331 (98%, M+Na⁺). ¹H NMR (400 MHz, acetone-d₆): δ 2.38 (d, J=1.8 Hz, 3H), 2.96 (t, J=6.5 Hz, 2H), 3.03 (br s, 1H), 3.55 (t, J=6.5 Hz, 2H), 7.21 (m, 5H), 7.61 (ddd, J=8.2, 2.5, & 1.9 Hz, 1H), 8.05 (ddd, J=8.2, 2.5, & 1.9 Hz, 1H), 8.42 (d, J=8.2 Hz, 1H), 8.72 (d, J=5.6 Hz, 1H).

### Pyrimidine 7 (7: R¹═R²=Me):

MALDI (matrix: DHB): m/z 308 (100%, M⁺+1), 330 (66%, M+Na⁺), 346 (80%, M+K⁺). NMR (400 MHz, acetone-d₆): δ 2.25 (s, 3H), 2.71 (s, 3H), 2.93 (t, J=6.8 Hz, 2H), 3.42 (t, J=6.8 Hz, 2H), 7.13 (m, 5H), 7.41 (ddd, J=8.1, 5.5, & 2.1 Hz, 1H), 7.85 (ddd, J=8.1, 5.5, & 2.1 Hz, 1H), 8.1 (d, J=8.1 Hz, 1H), 8.72 (d, J=5.5 Hz, 1H).

### Example 3

### Synthesis of 4-amino-5-cyano-6-phenyl-2(2-pyridinyl)pyrimidine (8)

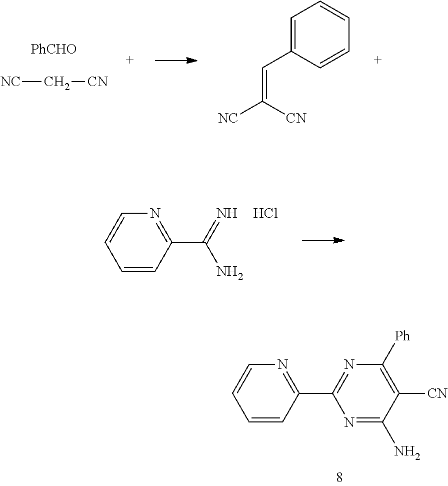

Synthesis of 4-amino-5-cyano-6-phenyl-2(2-pyridinyl)pyrimidine was synthesized following the procedure delineated by Peters, J-U. et al. (Bioorg. Med. Chem. Lett., 2004, 14, 1491-93.)

Pyrimidine 8: MALDI (matrix: DHB): m/z 274 (100%, M⁺+1), 296 (48%, M+Na⁺), 312 (70%, M+K⁺). ¹H NMR (400 MHz, acetone-d₆): δ 7.8 (m, 4H), 8.2 (app t, J=7.6 Hz, 2H), 8.58 (ddd, J=8.2, 5.5, & 2.1 Hz, 2H), 8.82 (ddd, J=8.2, 5.5, & 2.1 Hz, 1H), 9.2 (br s, 2H).

### Example 4

### Synthesis of 9-19

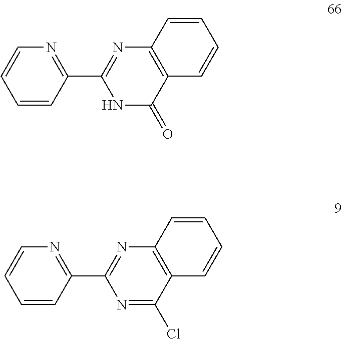

Quinazolines 66 (CAS# 28594-60-7) and 9 (CAS# 91748-47-9) were prepared according to the procedure of Flanagan, S. P. et al. (Tetrahedron, 2005, 61, 9808-21.)

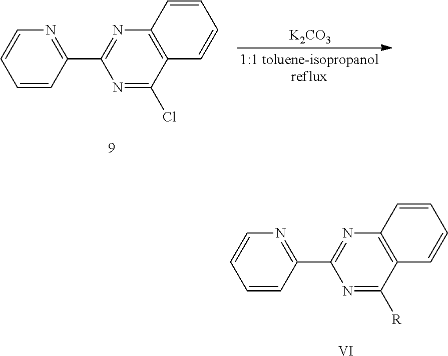

2-(Pyridin-2-yl)quinazolines of formula VI were prepared by nucleophilic displacement reaction using the appropriate amine.

Quinazoline 10 (10: R═—HNCH₂CH₂Ph):

MALDI (matrix: DHB): m/z 327 (100%, M⁺+1), 349 (26%, M+Na⁺). ¹H NMR (400 MHz, CDCl₃): δ 3.02 (t, J=7.6 Hz, 2H), 3.99 (td, J=12.4, 6.8 Hz, 2H), 6.47 (br t, 1H), 7.23 (m, 2H), 7.29 (m, 2H), 7.34 (m, 2H), 7.66 (t, J=7.6 Hz, 1H), 7.73 (d, J=8.4 Hz, 1H), 7.84 (ddd, J=8.4, 7.6, & 1.6 Hz, 1H), 8.06 (d, J=8.0 Hz, 1H), 8.63 (d, J=8.4 Hz, 1H), 8.81 (ddd, J=8.4, 1.6, & 0.8 Hz, 1H).

Quinazoline 11 (11: R═—HNCH₂CH₂NHPh):

MALDI (matrix: DHB): m/z 342 (100%, M⁺+1), 364 (95%, M+Na⁺). ¹H NMR (400 MHz, acetone-d₆): δ 3.71 (m, 4H), 4.13 (br s, 2H), 6.55 (t, J=7.6 Hz, 1H), 6.73 (m, 2H), 7.13 (m, 2H), 7.51 (m, 2H), 7.93 (m, 3H), 8.37 (m, 1H), 8.6 (app d, J=8.4 Hz, 1H), 8.83 (m, 1H).

Quinazoline 12 (11: R═—HNCH₂CH₂(3-indolyl)):

MALDI (matrix: DHB): m/z 366 (80%, M⁺+1), 388 (100%, M+Na⁺). ¹H NMR (400 MHz, acetone-d₆): δ 2.7 (t, J=7.5 Hz, 2H), 2.9 (t, J=7.5 Hz, 2H), 4.2 (br s, 1H), 6.9 (m, 3H), 7.26 (m, 1H), 7.36 (m, 2H), 7.85 (m, 2H), 8.1 (m, 1H), 8.6 (m, 1H), 8.8 (m, 1H), 10.03 (br s, 1H).

Quinazoline 13:

MALDI (matrix: DHB): m/z 403 (100%, M⁺+1), 425 (35%, M+Na⁺). ¹H NMR (400 MHz, CDCl₃): δ 2.12 (td, J=7.1 & 6.6 Hz, 2H), 3.42 (t, J=7.1 Hz, 2H), 4.1 (t, J=6.6 Hz, 1H), 7.28 (m, 10H), 7.47 (ddd, J=7.5, 6.1, 1.6 Hz, 2H), 7.87 (ddd, J=8.2, 7.5, 1.8 Hz, 1H), 7.81 (ddd, J=8.2, 1.8, 1.6 Hz, 1H), 7.86 (ddd, J=8.2, 7.4, 1.9 Hz, 1H), 8.08 (ddd, J=8.2, 5.5, 1.6 Hz, 1H), 8.91 (ddd, J=6.1, 5.5, 1.9 Hz, 2H).

Quinazoline 14 (14: R═4-(4-methoxyphenyl)piperazinyl):

MALDI (matrix: DHB): m/z 398 (100%, M⁺+1), 420 (15%, M+Na⁺). ¹H NMR (400 MHz, CDCl₃): δ 3.34 (m, 4H), 3.79 (s, 3H), 4.05 (m, 4H), 6.9 (m, 2H), 7.01 (m, 2H), 7.39 (m, 1H), 7.5 (m, 1H), 7.8 (m, 1H), 7.86 (m, 1H), 7.98 (d, J=7.6 Hz, 1H), 8.21 (d, J=9.2 Hz, 1H), 8.6 (d, J=8.0 Hz, 1H), 8.89 (m, 1H).

Quinazoline 15 (15: R=4-(3,4-dichlorophenyl)piperazinyl):

MALDI (matrix: DHB): m/z 437 (100%, M⁺+1). ¹H NMR (400 MHz, CD₃CN): δ 4.2 (m, 6H), 4.8 (m, 2H), 7.7 (dd, J=7.6, 1.2 Hz, 1H), 7.9 (t, J=1.2 Hz, 1H), 8.17 (d, J=8.4 Hz, 1H), 8.23 (m, 1H), 8.36 (app t, J=7.6 Hz, 1H), 8.61 (aap t, J=7.6 Hz, 1H), 8.75 (app t, J=7.6 Hz, 1H), 8.81 (ddd, J=8.4, 7.6, 1.2 Hz), 9.36 (d, J=7.6 Hz, 1H), 9.54 (dd, J=2.2, 1.2 Hz, 1H).

Quinazoline 16 (16: R=4-(4-toluenesulfonyl)piperazinyl):

MALDI (matrix: DHB): m/z 446 (100%, M⁺+1), 468 (96%, M+Na⁺). ¹H NMR (400 MHz, CDCl₃): δ 2.41 (s, 3H), 3.21 (m, 4H), 3.97 (m, 4H), 7.29 (m, 4H), 7.63 (m, 4H), 7.84 (m, 1H), 8.21 (d, J=8.2 Hz, 1H), 8.55 (d, J=8.2 Hz, 1H), 8.83 (m, 1H).

Quinazoline 17 (17: R=1-phenyl-1,3,7-triaza[5.4.0]bicyclodecan-7-yl;):

MALDI (matrix: DHB): m/z 437 (100%, M⁺+1), 459 (10%, M+Na⁺). ¹H NMR (400 MHz, CDCl₃): δ 1.98 (m, 2H), 3.1 (m, 2H), 4.2 (m, 2H), 4.5 (m, 2H), 4.8 (s, 2H), 6.42 (s, 1H), 6.7 (m, 3H), 7.15 (m, 2H), 7.42 (m, 1H), 7.46 (m, 2H), 7.8 (m, 2H), 7.98 (d, J=7.6 Hz, 1H), 8.3 (d, J=7.8 Hz, 1H), 8.6 (d, J=5.5 Hz, 1H), 8.97 (m, 1H).

Quinazoline 18 (18: R=4-(4-chlorophenyl)-4-hydroxy-piperidin-1-yl):

MALDI (matrix: DHB): m/z 417 (100%, M⁺+1), 440 (60%, M+Na⁺). ¹H NMR (400 MHz, CDCl₃): δ 1.98 (m, 2H), 2.31 (m, 2H), 2.8 (br s, 1H), 3.77 (m, 2H), 4.4 (m, 2H), 7.32 (m, 4H), 7.43 (m, 2H), 7.67 (m, 1H), 7.81 (m, 1H), 7.84 (d, J=7.6 Hz, 1H), 8.17 (d, J=7.6 Hz, 1H), 8.59 (d, J=7.6 Hz, 1H), 8.82 (m, 1H).

Quinazoline 19 (19: R=1,2,3,4-tetrahydroisoquinolin-2-yl):

MALDI (matrix: DHB): m/z 339 (100%, M⁺+1), 361 (30%, M+Na⁺). ¹H NMR (400 MHz, CDCl₃): δ 3.2 (m, 2H), 4.15 (m, 2H), 5.03 (s, 2H), 7.37 (m, 2H), 7.41 (m, 1H), 7.72 (t, J=5.5 Hz, 1H), 7.83 (m, 1H), 8.01 (d, J=5.5 Hz, 1H), 8.2 (d, J=7.6 Hz, 1H), 8.61 (d, J=5.5 Hz, 1H), 8.84 (m, 1H).

### Example 5

### Synthesis of 63

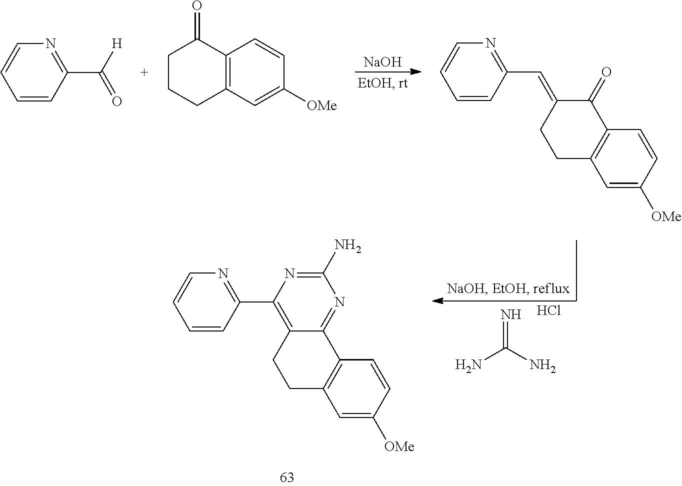

Compound 63 was synthesized according to the procedure reported by Nagarajan, S. et al. (Eur. J. Med. Chem., 2001, 42, 517-20.)

Ouinazoline 63: MALDI (matrix: DHB): m/z 305 (100%, M⁺+1). ¹H NMR (400 MHz, CDCl₃): δ 2.77 (m, 2H), 2.84 (m, 2H), 5.02 (br s, 2H), 7.02 (m, 1H), 7.16 (m, 2H), 7.61 (t, J=7.8 Hz, 1H), 8.17 (d, J=7.8 Hz, 1H), 8.36 (d, J=7.8 Hz, 1H), 8.43 (m, 1H).

### Example 6

### Synthesis of 64

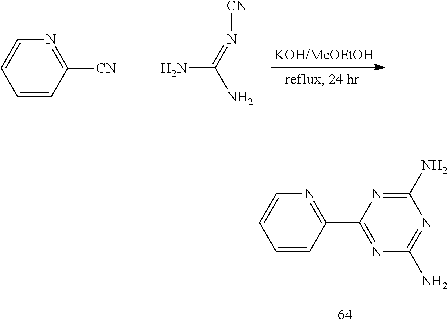

The symmetric triazine 64 was synthesized according the procedure reported by Shie, J. J. et al. (J. Org. Chem., 2003, 68, 1158-60.)

Triazine 64: MALDI (matrix: DHB): m/z 189 (100%, M⁺+1), 211 (97%, M+Na⁺). ¹H NMR (400 MHz, DMSO-d₆): δ 6.98 (br s, 2H), 6.86 (br s, 2H), 7.43 (dd, J=5.6, 1.5 Hz, 1H), 7.93 (app t, J=7.6 Hz, 1H), 8.2 (d, J=5.6 Hz, 1H), 8.63 (d, J=1.5 Hz, 1H).

### Example 7

### Synthesis of 65

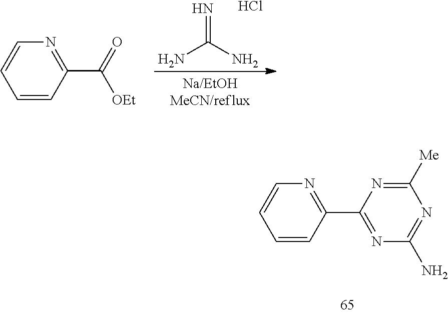

Triazine 65 was prepared as described Kelarev, V. I. et al. (Khimiya Geterotsiklicheskikh Soedinenii, 1988, (5), 674-80.)

Triazine 65: MALDI (matrix: DHB): m/z 187 (60%, M⁺+1), 209 (76%, M+Na⁺). ¹H NMR (400 MHz, DMSO-d₆): δ 2.86 (s, 3H), 6.86 (br s, 2H), 7.43 (dd, J=5.6, 1.5 Hz, 1H), 7.93 (app t, J=7.6 Hz, 1H), 8.2 (d, J=5.6 Hz, 1H), 8.63 (d, J=1.5 Hz, 1H).

### Example 8

### Synthesis of 43 and 44

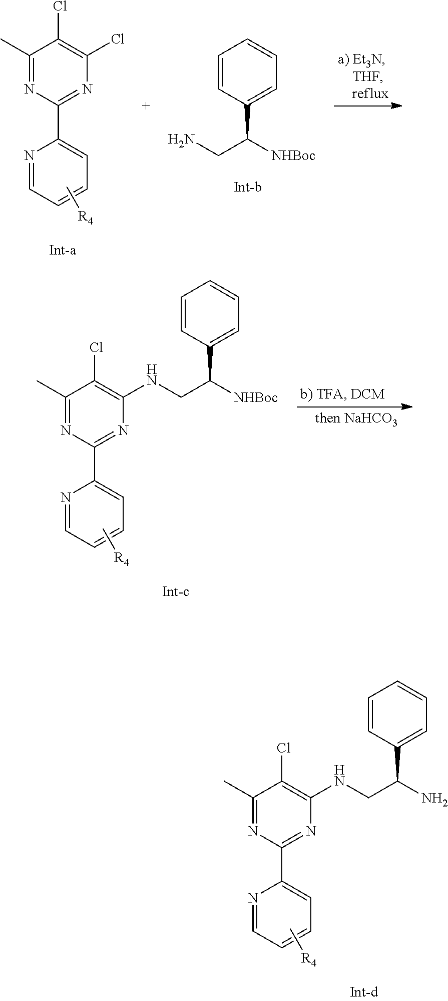

Compound Int-a (1 eq.) (synthesized according to Jeffrey B. Medwid, Rolf Paul et al. J. Med. Chem., (1990), 33, 1230-1241) and the amine Int-b (1.2 eq.) were dissolved in THF. Two equivalents of Et₃N was added. The mixture was heated at 50° C. for 12 h, at which point TLC analysis indicated complete transformation. The reaction mixture was cooled to room temparature and diluted with EA. After washed with water and brine. The organic phase was dried (Na₂SO₄), filtered and concentrated to give Int-c as a solid, which was used without purification in the next step.

Int-c was treated at RT with TFA:CH₂Cl₂ (3:1) for 1 h before being concentrated in vacuo. The resulting TFA salt was dissolved in EA, and then washed with aq NaHCO₃, and brine, dried (Na₂SO₄), filtered and concentrated. Purified by flash chromatography give Int-d.

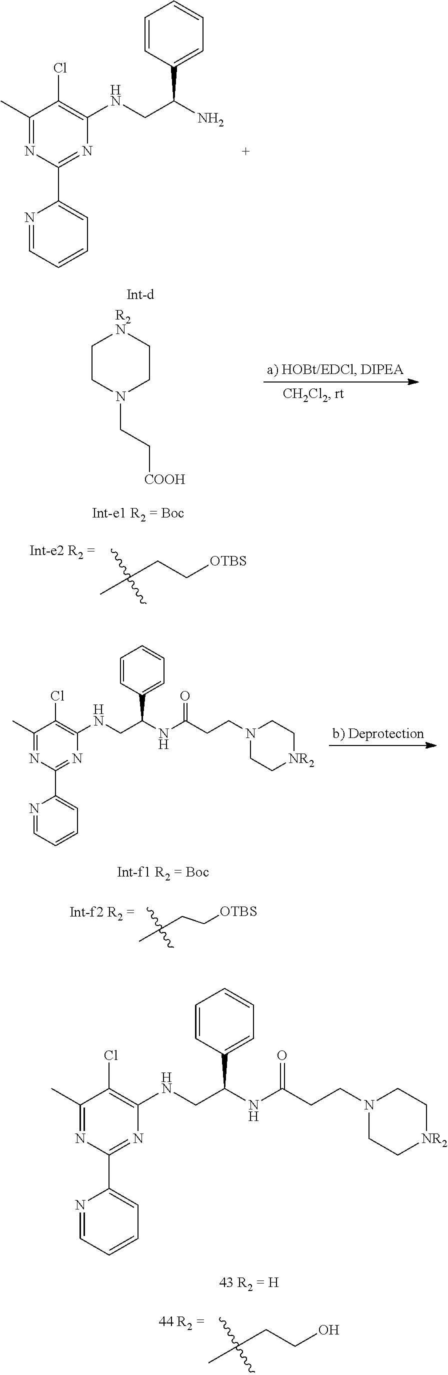

The acid Int-e1 (1.0 eq.) and the amine Int-d (1.2 eq.) were coupled using HOBt (1.2 eq.), EDCI (1.2 eq.) and DIPEA (2.0 eq.) in CH₂Cl₂ for 20 h. The reaction mixture was concentrated, taken up in ethyl acetate, washed twice with sat. NaHCO₃ and once with brine. The organic layer was dried on Na₂SO₄, filtered and concentrated to give Int-f1 as a yellowish solid, which was used in the next step without purification.

Deprotection of Int-f1 was carried out using the procedure as described for the deprotection of Int-c, to provide 43.

MS (ESI) m/z: 480.2 (MH⁺); HRMS calcd for C₂₅H₃₁ClN₇O (MH⁺) 480.2278: found 480.2278. ¹HNMR (300 MHz, CDCl₃) δ 9.37 (d, J=7.8 Hz, 1H), 8.81-8.77 (m, 1H), 8.43 (d, J=7.8 Hz, 1H), 7.82 (t, J=7.8 Hz, 1H), 7.42-7.39 (m, 4H), 7.38-7.31 (m, 2H), 6.32-6.24 (m, 1H), 5.38-5.28 (m, 2H), 4.14-4.03 (m, 1H), 4.01-3.89 (m, 1H), 2.79-2.64 (m, 4H), 2.61 (s, 3H), 2.52-2.45 (m, 2H), 2.41-2.23 (m, 6H).

To a solution of Int-f2 (77 mg, 0.121 mmol) in THF (1 mL) was added a 1.0 M TBAF solution in THF (242 μL, 0.242 mmol). The reaction mixture was stirred overnight at RT, and then diluted with EA. The solution was successively washed with water (2×) and brine. The organic phase was dried (Na₂SO₄) filtered and concentrated in vacuo. Purified by flash chromatograpy give a white solid 44 (54 mg, 84%).

MS (ESI) m/z: 524.3 (MH⁺); HRMS calcd for C₂₇H₃₅ClN₇O₂ (MH⁺) 524.2535: found 524.2531. ¹H NMR (300 MHz, CDCl₃) δ 9.36 (d, J=8.2 Hz, 1H), 8.79 9d, J=4.5 Hz, 1H), 8.46-8.40 (m, 1H), 7.83 (t, J=7.8 Hz, 1H), 7.44-7.30 (m, 5H), 6.29 (t, J=4.8 Hz, 1H), 5.37-5.29 (m, 1H), 4.13-3.91 (m, 2H), 3.56 (t, J=5.4 Hz, 2H), 2.60 (s, 3H), 2.55-2.48 (m, 2H), 2.46-2.14 (m, 12H).

### Example 9

### Synthesis of 45 and 46

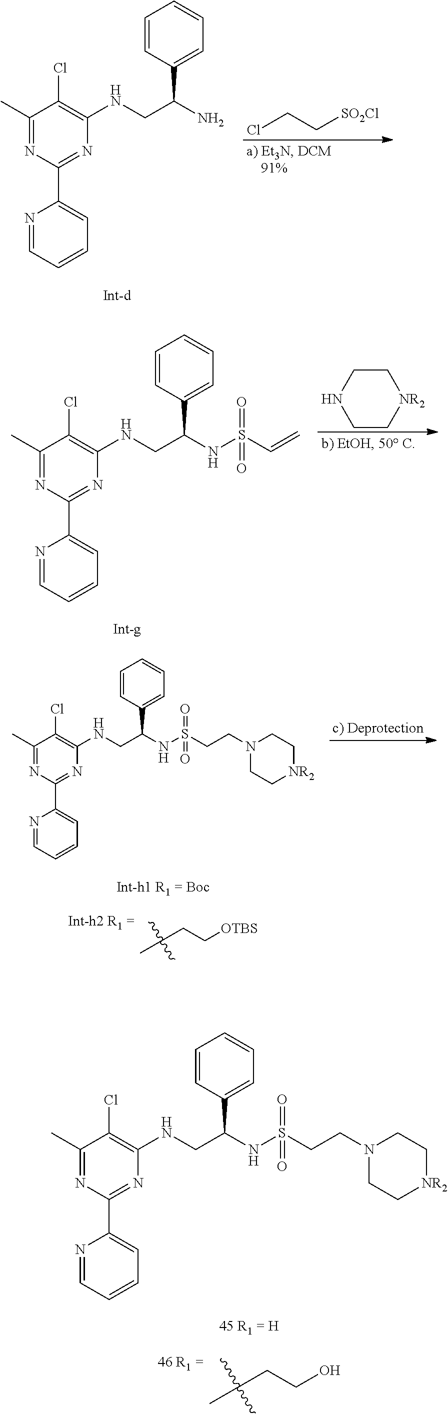

To a cooled solution (0° C.) of compound Int-d (594 mg, 1.75 mmol) and Et₃N (0.53 mL, 3.85 mmol) in CH₂Cl₂ (3 mL) was added 2-chloroethanesulfonyl chloride (627 mg, 3.85 mmol) with stirring. After addition, the mixture was stirred at rt for 5 h, and then was quenched with ice water. The reaction mixture was extracted with 3×5 mL portions of CH₂Cl₂, and the combined organic extracts were dried over Na₂SO₄, filtered, and concentrated under reduced pressure. Purified by flash chromatography give a white solid Int-g (689 mg, 91%).

¹H NMR (300 MHz, CDCl₃) δ 8.90-8.88 (m, 1H), 8.45 (d, J=8.1 Hz, 1H), 8.06 (d, J=7.5 Hz, 1H), 7.88-7.80 (m, 1H), 7.43-7.38 (m, 1H), 7.34-7.24 (m, 5H), 6.33 (dd, J=10.2, 16.5 Hz, 1H), 6.05-5.99 (m, 1H), 5.62-5.56 (m, 2H), 4.90-4.85 (m, 1H), 4.02-3.94 (m, 2H), 2.58 (s, 3H).

A solution of substituted piperazine (175 mg, 0.41 mmol) and sulfamide Int-g (379 g, 2.04 mmol) in absolute EtOH (5 mL) was heated at 50° C. for 16 h. Concentrated an purified by flash chromatography give a white solid Int-h1 (229 mg, 90%).

Int-h1: ¹H NMR (300 MHz, CDCl₃) δ 8.89-8.84 (m, 1H), 8.45 (d, J=7.5 Hz, 1H), 7.91 (d, J=7.2 Hz, 1H), 7.83 (t, J=7.5 Hz, 1H), 7.42-7.28 (m, 5H), 5.73 (t, J=6.0 Hz, 1H), 5.01-4.92 (m, 1H), 4.04-3.96 (m, 2H), 3.30-3.14 (m, 4H), 3.04-2.86 (m, 2H), 2.72-2.64 (m, 2H), 2.58 (s, 3H), 2.21-2.08 (m, 4H), 1.43 (s, 9H).

Int-h2: ¹H NMR (300 MHz, CDCl₃) δ 8.85-8.81 (m, 1H), 8.42 (d, J=7.8 Hz, 1H), 7.83-7.76 (m, 1H), 7.40-7.25 (m, 5H), 5.70 (t, J=6.0 Hz, 1H), 4.91 (bs, 1H), 4.08-3.90 (m, 2H), 3.64 (t, J=6.3 Hz, 2H), 3.01-2.87 (m, 2H), 2.71-2.62 (m, 2H), 2.55 (s, 3H), 2.40-2.15 (m, 8H), 0.84 (s, 9H), 0.00 (s, 6H).

Deprotection as previously described for Int-c provided 45.

MS (ESI) m/z: 516.1 (MH⁺); HRMS calcd for C₂₄H₃₁ ClN₇O₂S (MH⁺) 516.1943: found 516.1955. ¹H NMR (300 MHz, CDCl₃) δ 8.86-8.80 (m, 1H), 8.47-8.39 (m, 1H), 7.83 (t, J=4.2 Hz, 1H), 7.42-7.27 (m, 5H), 5.79 (t, J=5.7 Hz), 4.98-4.58 (m, 3H), 4.12-3.87 (m, 2H), 3.02-2.85 (m, 2H), 2.84-2.71 (m, 2H), 2.71-2.61 (m, 2H), 2.57 (s, 3H), 2.32-2.18 (m, 4H).

Deprotection as previously described for Int-f2 provided 46.

MS (ESI) m/z: 560.3 (MH⁺); HRMS calcd for C₂₆H₃₅ClN₇O₃S (MH⁺) 560.2205: found 560.2199. ¹H NMR (300 MHz, CDCl₃) δ 8.85 (d, J=4.8 Hz, 1H), 8.45 (d, J=7.8 Hz, 1H), 7.84 (t, J=7.5 Hz, 1H) 7.42-7.28 (m, 6H), 5.79 (t, J=5.7 Hz, 1H), 4.97-4.89 (m, 1H), 4.13-4.01 (m, 1H), 4.00-3.90 (m, 1H), 3.55 (t, J=5.4 Hz, 2H), 3.03-2.91 (m, 2H), 2.74-2.65 (m, 2H), 2.58 (s, 3H), 2.47-2.15 (m, 12H).

### Example 10

### Synthesis of 47 and 48

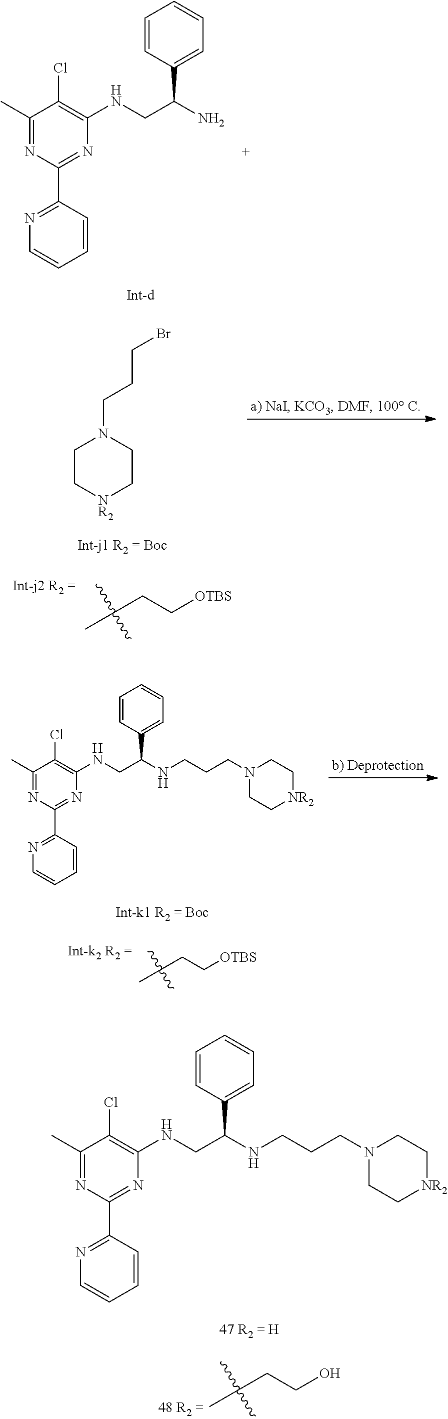

NaI (152 mg, 1.02 mmol) and anhydrous K₂CO₃ (140 mg, 1.02 mmol) were added to a solution of Int-d (115 mg, 0.34 mmol) and Int-j1 (156 mg, 0.51 mmol) in dry DMF (1 mL). After being stirred overnight at 100° C. for 24 h. The mixture was cooled to RT, and diluted with EA, washed with water and brine. The organic phase was dried (Na₂SO₄), filtered and concentrated. Purified by flash chromatograpy give a white solid Int-k1 (90 mg, 47%).

¹H NMR (300 MHz, CDCl₃) δ 8.81-8.77 (m, 1H), 8.39 (d, J=7.8 Hz, 1H), 7.82-7.75 (m, 1H), 7.42-7.28 (m, 5H), 6.13-6.04 (m, 1H), 3.99-3.87 (m, 2H), 3.75-3.61 (m, 1H), 3.44-3.34 (m, 4H), 2.68-2.56 (m, 5H), 2.44-2.28 (m, 6H), 1.75-1.62 (m, 2H), 1.45 (s, 9H).

Deprotection of Int-k1 was performed as previously described for Int-c to provide 47.

MS (ESI) m/z: 466.2 (MH⁺); HRMS calcd for C₂₅H₃₃ClN₇ (MH⁺) 466.2481: found 466.2496. ¹H NMR (300 MHz, CDCl₃) δ 8.79 (d, J=4.8 Hz, 1H), (8.42-8.36, m, 1H), 7.79 (dt, J=1.8, 7.5 Hz, 1H), 7.42-7.29 (m, 6H), 6.18-6.10 (m, 1H), 3.97-3.86 (m, 2H), 3.72-3.60 (m, 1H), 2.91-2.82 (m, 4H), 2.68-2.52 (m, 9H), 2.48-2.31 (m, 4H), 1.74-1.60 (m, 2H).

Deprotection of Int-k2 as previously described for Int-f2 provided 48.

MS (ESI) m/z: 510.2 (MH⁺); HRMS calcd for C₂₇H₃₇ClN₇O (MH⁺) 510.2743: found 510.2751. ¹H NMR (300 MHz, CDCl₃) δ 8.80 (d, J=4.8 Hz, 1H), 8.39 (d, J=7.8 Hz, 1H), 7.83-7.75 (m, 1H), 7.43-7.28 (m, 5H), 6.15-6.08 (m, 1H), 3.98-3.87 (m, 2H), 3.70-3.56 (m, 3H), 2.68-2.57 (m, 6H), 2.56-2.35 (m, 9H), 1.76-1.61 (m, 2H).

### Example 11

### Synthesis of 51 and 52

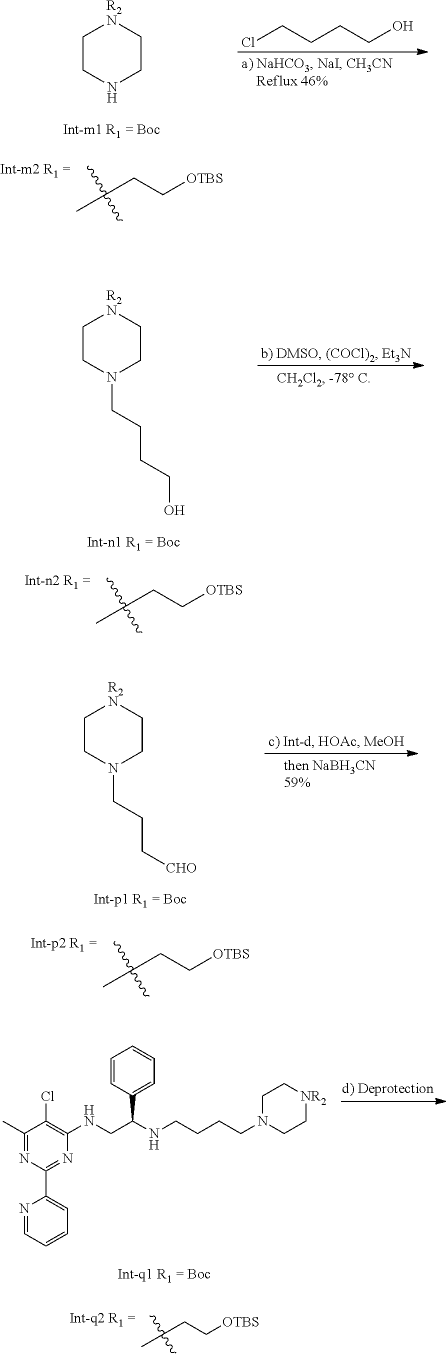

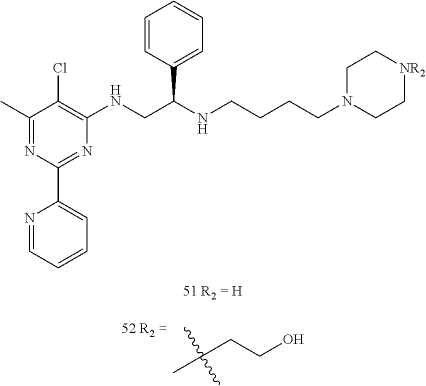

NaI (450 mg, 3.00 mmol) and anhydrous NaHCO₃ (1.512 g, 24.0 mmol) were added to a solution of the amine Int-m2 (1.468 g, 6.00 mmol) and 4-chlorobutan-1-ol (1.299 g, 12.0 mmol) in dry acetonitrile (12 mL). After being stirred overnight at 90° C. for 18 h. The mixture was cooled to RT, and diluted with EA, washed with water and brine. The organic phase was dried (Na₂SO₄), filtered and concentrated. Purified by flash chromatograpy give a yellowish oil Int-n2 (1.104 g, 58%).

DMSO (0.25 mL, 3.48 mmol) was added to a solution of oxalyl chloride (151 μL, 1.74 mmol) in CH₂Cl₂ (8 mL) at −78° C. under argon. After stirring for 20 min, alcohol Int-n2 (500 mg, 1.58 mmol) dissolved in CH₂Cl₂ (1 mL) was added dropwise (10 min). Stirring was continued for an additional 70 min. Et₃N (1.08 mL, 7.90 mmol) was added, and the reaction mixture was warmed to RT over 1.5 h. It was diluted with CH₂Cl₂, washed with saturated aq NaHCO₃. The organic phase was dried (Na₂SO₄), filtered and concentrated. Purified by flash chromatograpy give a yellowish oil Int-p2 (318 mg, 64%).

¹H NMR (300 MHz, CDCl₃) δ 9.77-9.74 (m, 1H), 3.75 (t, J=6.6 Hz, 1H), 3.65-2.40 (m, 12H), 2.35 (t, J=7.2 Hz, 2H), 1.90-1.77 (m, 2H), 0.89 (s, 9H), 0.05 (s, 6H).

To a solution of Int-d (85 mg, 0.25 mmol) and Int-p2 (79 mg, 0.25 mmol) in MeOH (0.7 mL) was added acetic acid (72 μL, 1.25 mmol) under argon. The reaction mixture was stirred for 10 min at RT, and then NaBH₃CN (47 mg, 0.75 mmol) was added at 0° C. After being stirred for 1 h, the reaction mixture was diluted with CH₂Cl₂, washed with water. The organic phase was dried (Na₂SO₄), filtered and concentrated. Purified by flash chromatograpy give a white solid Int-q2 (94 mg, 59%).

¹H NMR (300 MHz, CDCl₃) δ 8.75 (d, J=4.2 Hz, 1H), 8.34 (d, J=8.1 Hz, 1H), 7.75 (t, J=7.5 Hz, 1H), 7.39-7.24 (m, 5H), 6.11-6.03 (m, 1H), 3.99-3.82 (m, 2H), 3.79-3.66 (m, 3H), 2.65-2.39 (m, 13H), 2.38-2.25 (m, 2H), 1.50 (bs, 4H), 0.83 (s, 9H), 0.00 (s, 6H).

Deprotection of Int-q1 as previously described for Int-c provided 51.

MS (ESI) m/z: 480.2 (MH⁺); HRMS calcd for C₂₆H₃₄ClN₇ (MH⁺) 480.2637: found 480.2630. ¹H NMR (300 MHz, CDCl₃) δ 8.80 (d, J=4.2 Hz, 1H), 8.40 (d, J=8.1 Hz, 1H), 7.79 (dt, J=1.8, 7.5 Hz, 1H), 7.42-7.27 (m, 6H), 6.14-6.06 (m, 1H), 3.97-3.85 (m, 2H), 3.72-3.61 (m, 1H), 2.88 (t, J=4.8 Hz, 4H), 2.61 (s, 3H), 2.60-2.50 (m, 2H), 2.46-2.24 (m, 6H), 1.60-1.41 (m, 4H).

Deprotection of Int-q2 as previously described for Int-f2 provided 52.

MS (ESI) m/z: 524.2 (MH⁺); HRMS calcd for C₂₈H₃₉ClN₇O (MH⁺) 524.2899: found 524.2908. ¹H NMR (300 MHz, CDCl₃) δ 8.80 (d, J=4.8 Hz, 1H), 8.39 (d, J=7.8 Hz, 1H), 7.83-7.75 (m, 1H), 7.42-7.28 (m, 6H), 6.10-6.02 (m, 1H), 3.96-3.84 (m, 2H), 3.73-3.55 (m, 4H), 2.61 (s, 3H), 2.59-2.37 (m, 10H), 2.36-2.22 (m, 4H), 1.58-1.40 (m, 4H).

### Example 12

### Synthesis of 49, 53, 55, 57, 59, and 61

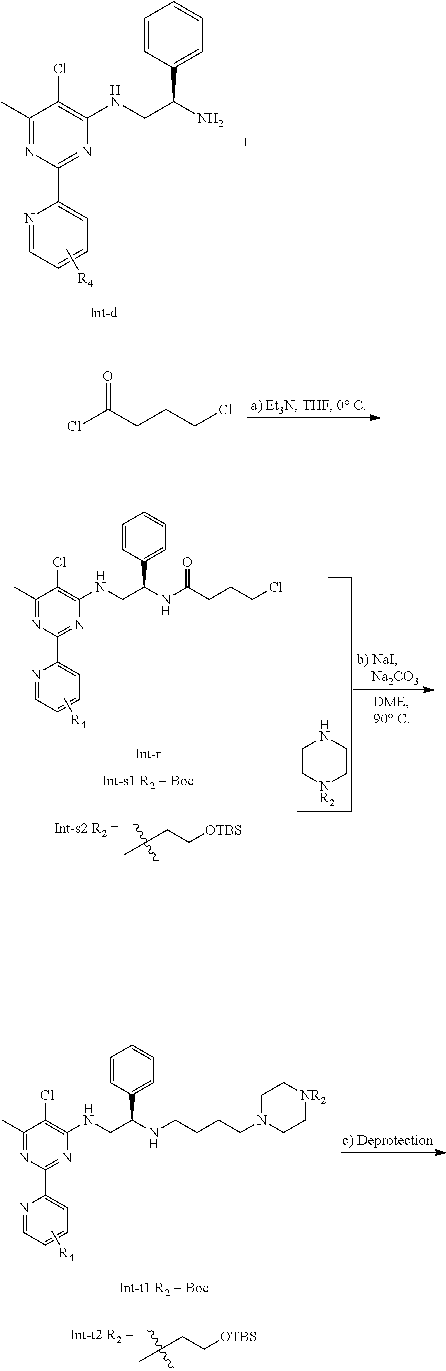

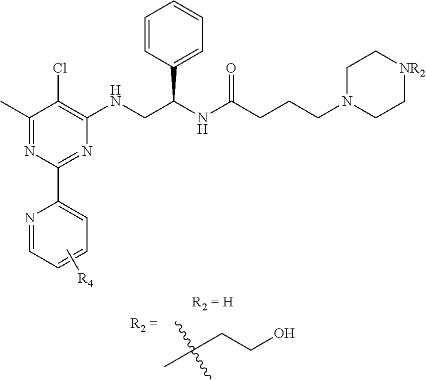

4-chlorobutanoyl chloride (2.0 eq) was added dropwisely to the solution of Int-d (1.0 eq.) and Et₃N (2.0 eq.) in anhydrous THF at 0° C. After being stirred for 0.5 h, the reaction mixture was diluted with EA, washed with brine. The organic phase was dried (Na₂SO₄), filtered and concentrated. Purified by flash chromatograpy give Int-r.

NaI (0.5 eq.) and anhydrous Na₂CO₃ (1.5 eq.) were added to a solution of the amine Int-s1 (1.05 eq.) and Int-r (1.0 eq.) in DME. After being stirred at 90° C. for 18 h. The mixture was cooled to RT, and diluted with EA, washed with water and brine. The organic phase was dried (Na₂SO₄), filtered and concentrated. Purified by flash chromatograpy give Int-t1.

Deprotection of Int-t1 as previously described for Int-c provided 49, 53, 55, 57, 59, and 61.

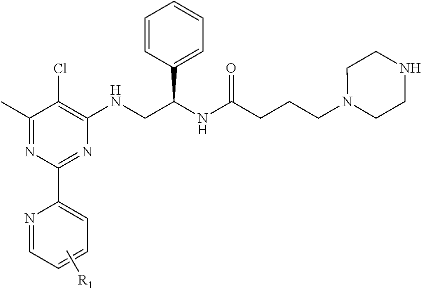

49 (R₁═H) MS (ESI) m/z: 494.1 (MH⁺); HRMS calcd for C₂₆H₃₃ClN₇O (MH⁺) 494.2430: found 494.2440. ¹H NMR (300 MHz, CDCl₃) δ 8.82 (d, J=4.5 Hz, 1H), 8.44 (d, J=7.8 Hz, 1H), 7.89-7.76 (m, 2H), 7.43-7.28 (m, 6H), 6.1 (t, J=6.0 Hz, 1H), 5.33-5.23 (m, 1H), 4.15-4.02 (m, 1H), 3.96-3.86 (m, 1H), 2.77 (t, J=4.8 Hz, 4H), 2.60 (s, 3H), 2.33-2.11 (m, 10H), 1.76-1.64 (m, 2H).

53 (R₁=6-Me) MS (ESI) m/z: 508.3 (MH⁺); HRMS calcd for C₂₇H₃₅ClN₇O (MH⁺) 508.2586: found 508.2572. ¹H NMR (300 MHz, CDCl₃) δ (8.21-8.10, m, 2H), 7.68 (t, J=7.8 Hz, 1H), 7.40-7.17 (m, 5H), 6.21 (t, J=5.4 Hz, 1H), 5.24-5.14 (m, 1H), 4.17-4.03 (m, 1H), 3.75-3.63 (m, 1H), 3.50 (bs, 3H), 2.72 (bs, 3H), 2.66 (s, 3H), 2.53 (s, 3H), 2.18 (bs, 4H), 2.09-1.96 (m, 4H), 1.64-1.50 (m, 2H).

55 (R₁=4-Cl) MS (ESI) m/z: 528.1 (MH⁺); HRMS calcd for C₂₆H₃₂Cl₂N₇O (MH⁺) 528.2040: found 528.2027. ¹H NMR (300 MHz, CDCl₃) δ 8.70 (d, J=5.4 Hz, 1H), 8.47-8.43 (m, 1H), 7.65 (d, J=6.9 Hz, 1H), 7.42-7.28 (m, 5H), 6.24-6.15 (m, 1H), 5.33-5.23 (m, 1H), 4.14-4.01 (m, 1H), 3.96-3.85 (m, 1H), 2.82-2.72 (m, 4H), 2.59 (s, 3H), 2.38-2.13 (m, 10H), 1.79-1.66 (m, 2H).

57 (R₁=5-Br) MS (ESI) m/z: 572.0 (MH⁺); HRMS calcd for C₂₆H₃₂BrClN₇O (MH⁺) 572.1535: found 572.1541. ¹H NMR (300 MHz, CDCl₃) δ 8.83 (d, J=2.1 Hz, 1H), 8.38-8.31 (m, 1H), 8.00-7.92 (m, 1H), 7.83 (d, J=6.9 Hz, 1H), 7.41-7.25 (m, 5H), 6.30-6.20 (m, 1H), 5.31-5.21 (m, 1H), 4.12-3.96 (m, 1H), 3.92-3.79 (m, 1H), 3.31 (bs, 3H), 2.80 (bs, 3H), 2.57 (s, 3H), 2.43-2.09 (m, 8H), 1.81-1.62 (m, 2H).

59 (R₁=3-Cl) MS (ESI) m/z: 528.2 (MH⁺); HRMS calcd for C₂₆H₃₂Cl₂N₇O (MH⁺) 528.2040: found 528.2036. ¹H NMR (300 MHz, CDCl₃) δ¹H NMR (300 MHz, CDCl₃) δ 8.62 (d, J=4.8 Hz, 1H), 7.88-7.82 (m, 1H), 7.42 (d, J=7.2 Hz, 1H), 7.39-7.24 (m, 5H), 6.24 (t, J=5.4 Hz, 1H), 5.29-5.19 (m, 1H), 4.10-3.97 (m, 1H), 3.85-3.73 (m, 1H), 2.84-2.72 (m, 3H), 2.57 (s, 3H), 2.44 (s, 4H), 2.37-2.09 (m, 8H), 1.78-1.64 (m, 2H).

61 (R₁=4-Me) MS (ESI) m/z: 508.3 (MH⁺); HRMS calcd for C₂₇H₃₅ClN₇O (MH⁺) 508.2586: found 508.2582. ¹H NMR (300 MHz, CDCl₃) δ 8.60 (d, J=4.8 Hz, 1H), 8.23 (s, 1H), 8.03 (d, J=6.9 Hz, 1H), 7.38-7.21 (m, 5H), 7.20-7.15 (m, 1H), 6.05 (t, J=6.0 Hz, 1H), 5.29-5.19 (m, 1H), 4.15-4.02 (m, 1H), 3.86-3.76 (m, 2H), 2.71 (t, J=4.8 Hz, 4H), 2.54 (s, 3H), 2.43 (s, 3H), 2.28-2.05 (m, 8H), 1.70-1.57 (2H).

### Example 13

### Synthesis of 50, 54, 56, 58, 60 and 62

Deprotection of Int-t2 as previously described for Int-f2 provided 50, 54, 56, 58, 60 and 62.

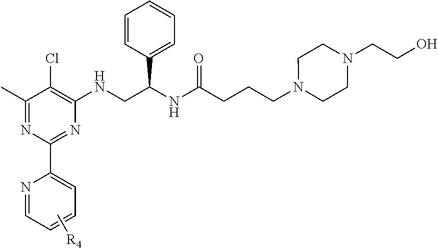

50 (R₁═H) MS (ESI) m/z: 538.2 (MH⁺); HRMS calcd for C₂₈H₃₇ClN₇O₂ (MH⁺) 538.2692: found 538.2700. 8.83-8.79 (m, 1H), 8.44 (d, J=7.8 Hz, 1H), 7.84 (dt, J=1.5, 7.5 Hz, 1H), 7.76 (d, J=6.6 Hz, 1H), 7.42-7.28 (m, 5H), 6.06 (t, J=5.4 Hz, 1H), 5.32-5.23 (m, 1H), 4.15-4.03 (m, 1H), 3.95-3.86 (m, 1H), 3.57 (t, J=5.4 Hz, 2H), 2.61 (s, 3H), 2.51-2.11 (m, 12H), 1.76-1.63 (m, 2H).

54 (R₁=6-Me) MS (ESI) m/z: 552.5 (MH⁺); HRMS calcd for C₂₉H₃₉ClN₇O₂ (MH⁺) 552.2848: found 552.2834. ¹H NMR (300 MHz, CDCl₃) δ 8.22 (d, J=7.8 Hz, 1H), 8.06 (d, J=6.9 Hz, 1H), 7.73 (t, J=7.8 Hz, 1H), 7.45-7.24 (m, 5H), 6.08 (t, J=5.7 Hz, 1H), 5.31-5.21 (m, 1H), 4.27-4.14 (m, 1H), 3.82-3.69 (m, 1H), 3.57 (t, J=5.4 Hz, 2H), 2.71 (s, 3H), 2.62-2.51 (m, 6H), 2.46 (t, J=5.4 Hz, 3H), 2.38 (bs, 4H), 2.26 (bs, 4H), 2.15-2.02 (m, 4H), 1.69-1.55 (m, 2H).

56 (R₁=4—Cl) MS (ESI) m/z: 572.1 (MH⁺); HRMS calcd for C₂₈H₃₆Cl₂N₇O₂ (MH⁺) 572.2302: found 572.2287. ¹H NMR (300 MHz, CDCl₃) δ 8.70 (d, J=5.1 Hz, 1H), 8.47-8.44 (m, 1H), 7.58 (d, J=7.5 Hz, 1H), 7.44-7.28 (m, 6H), 6.15 (t, J=5.4 Hz, 1H), 5.34-5.24 (m, 1H), 4.15-4.02 (m, 1H), 3.96-3.86 (m, 1H), 3.58 (t, J=5.4 Hz, 2H), 2.60 (s, 3H), 2.52-2.13 (m, 12H), 1.78-1.65 (m, 2H).

58 (R₁=5-Br) MS (ESI) m/z: 616.2 (MH⁺); HRMS calcd for C₂₈H₃₆BrClN₇O₂ (MH⁺) 616.1797: found 616.1796. ¹H NMR (300 MHz, CDCl₃) δ 8.85-8.81 (m, 1H), 8.35 (d, J=8.4 Hz, 1H), 8.00-7.92 (m, 2H), 7.43-7.25 (m, 5H), 6.26 (t, J=5.4 Hz, 1H), 5.30-5.21 (m, 1H), 4.08-3.96 (m, 1H), 3.94-3.82 (m, 1H), 3.73-3.53 (m, 4H), 2.64-2.38 (m, 12H), 2.37-2.18 (m, 4H), 1.84-1.69 (m, 2H).

60 (R₁=3-Cl) MS (ESI) m/z: 572.3 (MH⁺); HRMS calcd for C₂₈H₃₆Cl₂N₇O₂ (MH⁺) 572.2302: found 572.2298. ¹H NMR (300 MHz, CDCl₃) δ 8.65-8.60 (m, 1H), 7.88-7.81 (m, 1H), 7.41-7.28 (m, 6H), 6.20 (t, J=5.4 Hz, 1H), 5.30-5.20 (m, 1H), 4.10-3.97 (m, 1H), 3.86-3.75 (m, 1H), 3.58 (t, J=5.4 Hz, 2H), 2.58 (s, 3H), 2.52-2.12 (m, 12H), 1.77-1.64 (m, 2H).

62 (R₁=4-Me) MS (ESI) m/z: 552.4 (MH⁺); HRMS calcd for C₂₉H₃₉ClN₇O₂ (MH⁺) 552.2848: found 552.2823. ¹H NMR (300 MHz, CDCl₃) δ 8.64 (d, J=4.8 Hz, 1H), 8.27 (s, 1H), 8.05 (d, J=7.2 Hz, 1H), 7.43-7.25 (m, 5H), 7.24-7.19 (m, 1H), 6.04 (t, J=5.4 Hz, 1H), 5.33-5.23 (m, 1H), 4.22-4.09 (m, 1H), 3.89-3.79 (m, 1H), 3.58 (t, J=5.4 Hz), 2.70 (s, 4H), 2.50 (s, 3H), 2.51-2.44 (m, 5H), 2.44-2.22 (m, 7H), 2.21-2.10 (m, 4H), 1.73-1.61 (m, 2H).

### Example 14

### Biological Assays

### Materials

[³H]-thymidine was obtained from Perkin Elmer (Wellesley, Mass.). MetAP1 polyclonal antibodies were a generous gift from Dr. Y-H Chang at St. Louis University School of Medicine. A MetAP2 monoclonal antibody was generated with the help of Dr. J. E. K. Hildreth (Department of Pharmacology, Johns Hopkins School of Medicine). 14-3-3γ monoclonal antibody (clone HS23) was obtained from Novus Biologicals (Littleton, Colo.). PARP and active caspase-3 monoclonal antibodies were obtained from BD Bioscience (San Diego, Calif.). Tubulin, Cyclin B1, cdc2/Cdk1 monoclonal antibodies and pro-caspase-3 antibody were obtained from Santa Cruz Inc. (Santa Cruz, Calif.). DMSO, Paclitaxel, Thymidine, Propidium Iodide, DNase-free RNase and β-actin monoclonal antibody were obtained from Sigma Aldrich. (St. Louis. MO). SuperFect reagents were obtained from Qiagen (Valencia, Calif.). Oligofectamine and TRIZOL reagent were purchased from Invitrogen (Carlsbad, Calif.).

### MetAP Enzyme Assay

Full-length and truncated HsMetAP1 were generated as described in Addlagatta, A., Hu, X., Liu, J. O. & Matthews, B. W. (2005) Biochemistry 44, 14741-14749. Recombinant HsMetAP2 was produced according to Turk et al. (1999) Chem. Biol. 6, 823-833. MetAP enzymatic assay was carried out as described previously (Zhou, Y., et al. (2000) Anal. Biochem. 280, 159-165).

### Double Thymidine Synchronization

Cultured HeLa and HT-1080 cells were synchronized according to Hirota et al. (2003) Cell 114, 585-598. Briefly, 1.5×10⁵ cells were seeded in 6-well plate and treated with 2 mM Thymidine for 20 hrs before released with fresh medium for 8 hrs. 2 mM Thymidine was then added as the second arrest for 14 hrs before released by fresh medium with respective compounds.

### Cell Cycle Analysis

Cultured cells were trypsinized, fixed with 70% ethanol at 4° C. overnight before being stained with propidium iodide using Staining solution (20 μg/mL PI, 200 μg/mL DNase-free RNase A and 0.1% (v/v) Triton X-100 in PBS) prepared freshly. DNA contents were analyzed using the FACScan (Becton Dickinson, San Jose) (Kanzawa, T., et al. (2003) Br. J. Cancer 89, 922-92). Data were analyzed by CellQuest software (Becton Dickinson).

siRNA Transfection

siRNAs duplexes were obtained from Dharmacon, Inc (Lafayette, Colo.). The following siRNA targeting (sense) sequences were selected: MetAP1 siRNA: 5′-GGCCAGUGCCAAGUUAUAU-dTdT-3′, corresponding to bases 317-336 in the open reading frame (ORF) of the MetAP1 mRNA. MetAP2 and scrambled control siRNA duplexes were adopted from Bernier et al. (2005) J. Cell Biochem. 95, 1191-1203. MetAP2 siRNA: 5′-GAAGAGAUUUGGAAUGAUU-dTdT-3′, corresponds to bases 521-540 in the ORF of the MetAP2 mRNA. The scrambled control siRNA duplex sequence was 5′-AUUAGACUCUUCAUGGAAA-dTdT-3′. 1.5×10⁵ HeLa cells were seeded into 6-well plated before transfected by Oligofectamine (Invitrogen) according to manufacturer's instructions for 6 hrs. The final siRNA concentration was 100 nM. Double-thymidine synchronization was then initiated.

All references cited herein, whether in print, electronic, computer readable storage media or other form, are expressly incorporated by reference in their entirety, including but not limited to, abstracts, articles, journals, publications, texts, treatises, technical data sheets, interne web sites, databases, patents, patent applications, and patent publications.

A number of embodiments of the invention have been described. Embodiments herein include those recited alone or in combination with other delineated embodiments herein. Nevertheless, it will be understood that various modifications may be made without departing from the spirit and scope of the invention. Accordingly, other embodiments are within the scope of the following claims.

1. A compound of formula (I): 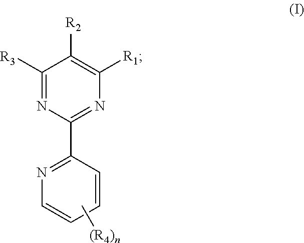
or a pharmaceutically acceptable salt thereof,
wherein,
R₁ is NR_{A}R_{A}, OR_{A}, SR_{A}, optionally substituted heterocyclic, optionally substituted cycloalkyl, or hal;
each R_{A} is independently H, an optionally substituted alkyl, or an optionally substituted aralkyl;
R₂ is H, an optionally substituted alkyl, cyano, nitro, azido, or hal;
R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;
or R₂ and R₃ can be taken together to form an optionally substituted aryl;
each R₄ is independently an optionally substituted alkyl or hal; and
n is 0, 1, 2, 3, or 4.

2. The compound of claim 1, wherein R₁ is NR_{A}R_{A}, OR_{A}, or SR_{A}.

3. The compound of claim 2, wherein R₁ is NH₂, NH—CH₂—CH₂—R_{B}, NH—CH₂—CH₂—NH—R_{B}, O—CH₂—CH₂—R_{B}, S—CH₂—CH₂—R_{B}, S—CH₂—R_{B}, 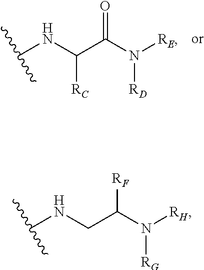
each of which may be optionally substituted.

4. The compound of claim 3, wherein each R_{B} is independently an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl.

5-14. (canceled)

15. The compound of claim 1, of formula (II): 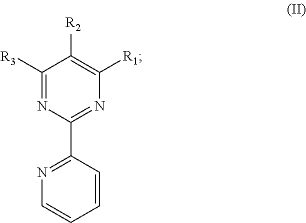
or a pharmaceutically acceptable salt thereof,
wherein,
R₁ is NR_{A}R_{A}, OR_{A}, SR_{A}, or hal;
each R_{A} is independently H, an optionally substituted alkyl, or an optionally substituted aralkyl;
R₂ is H, an optionally substituted alkyl, cyano, nitro, azido, or halo; and
R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl.

16. The compound of claim 15, wherein R₂ is H, an optionally substituted alkyl, cyano, nitro, azido, or halo; and R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl.

17-19. (canceled)

20. The compound of claim 15, selected from:

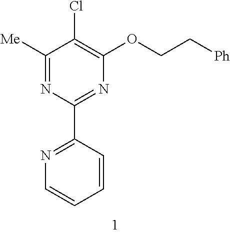

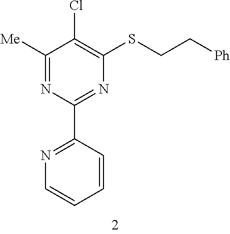

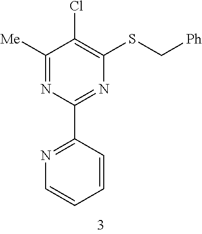

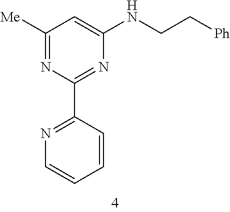

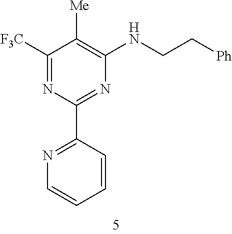

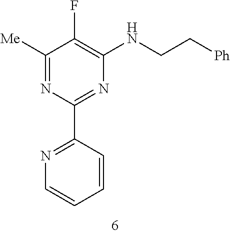

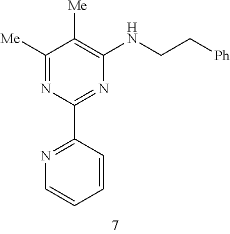

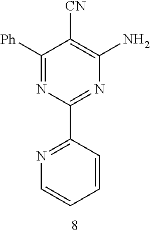

21. The compound of claim 1, of formula (III): 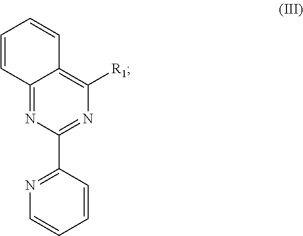
or a pharmaceutically acceptable salt thereof,
wherein,
R₁ is NR_{A}R_{A}, OR_{A}, SR_{A}, optionally substituted heterocyclic, optionally substituted cycloalkyl, or hal;
each R_{A} is independently H, an optionally substituted alkyl, or an optionally substituted aralkyl.

22-24. (canceled)

25. The compound of claim 21, selected from:

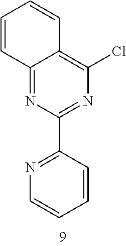

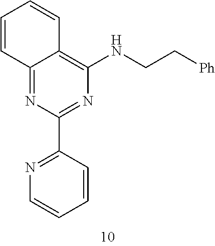

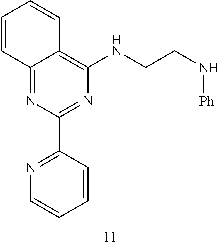

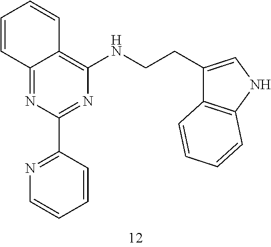

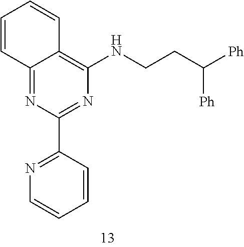

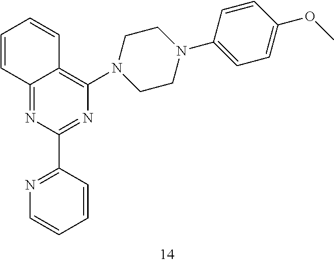

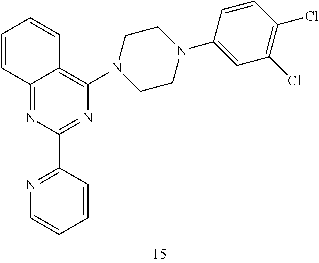

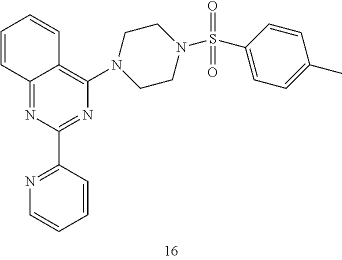

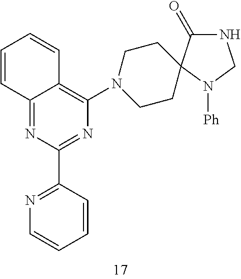

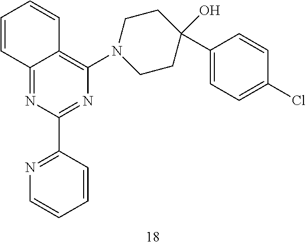

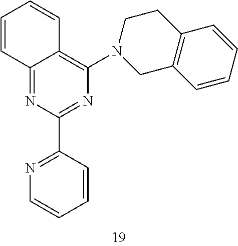

26. The compound of claim 1, of formula (IV): 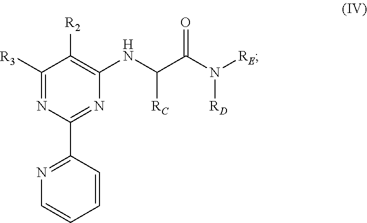
or a pharmaceutically acceptable salt thereof,
wherein,
R_{C} is an optionally substituted alkyl or an optionally substituted aralkyl;
R_{D} and R_{E} are each independently H, an optionally substituted alkyl, an optionally substituted aralkyl, or an optionally substituted hetero-aralkyl;
R₂ is H, an optionally substituted alkyl, cyano, nitro, azido, or halo; and
R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl.

27-32. (canceled)

33. The compound of claim 26, selected from:

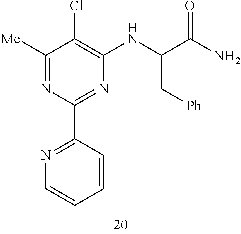

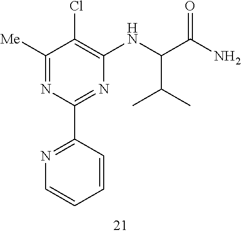

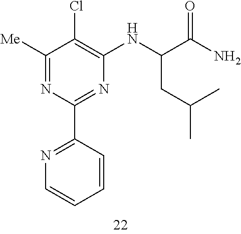

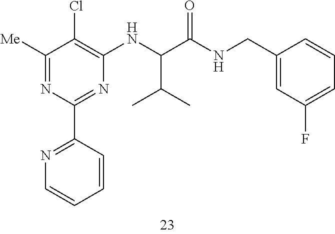

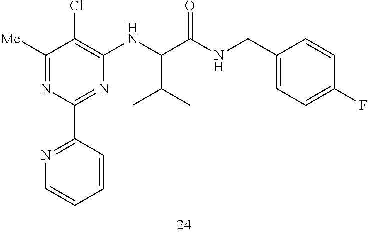

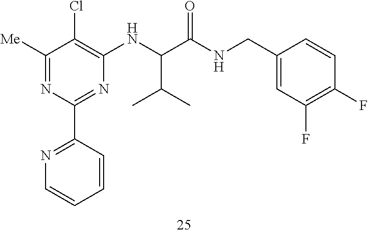

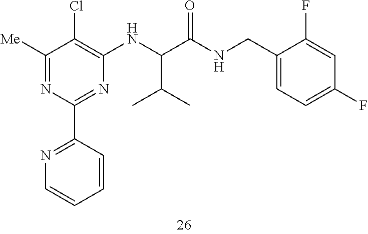

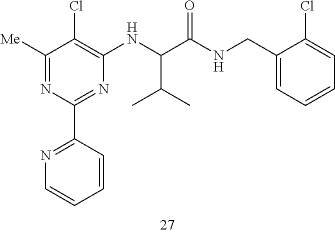

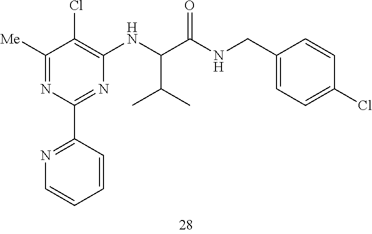

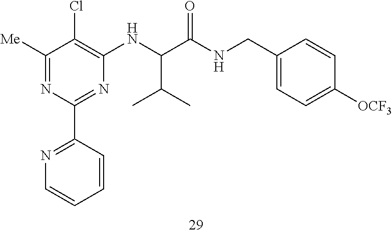

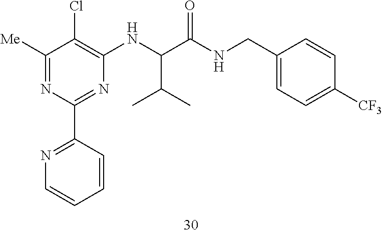

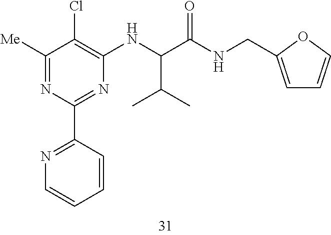

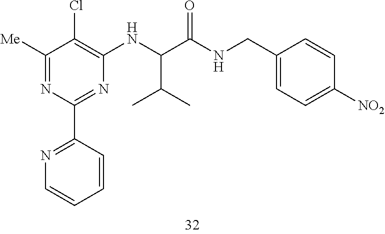

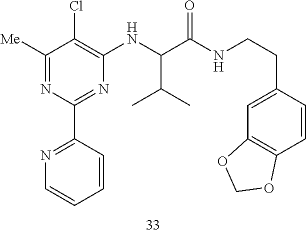

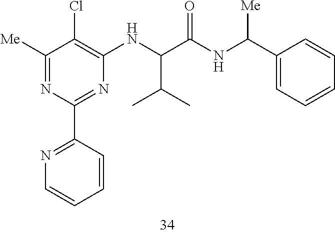

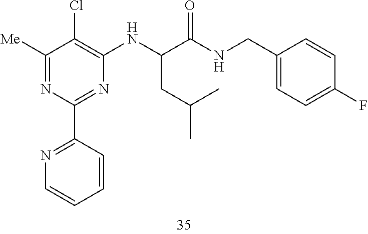

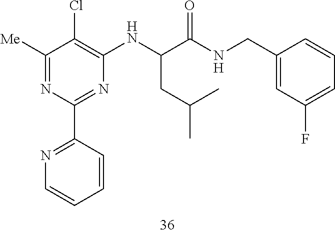

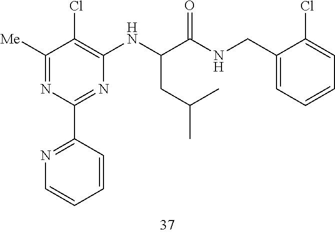

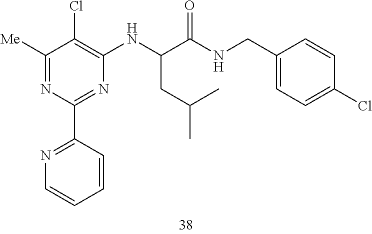

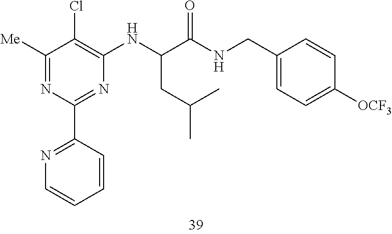

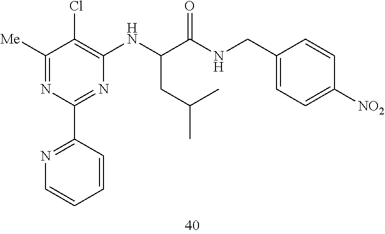

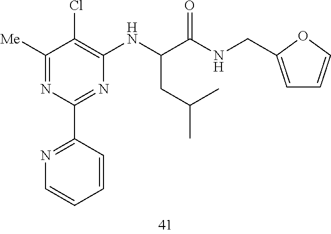

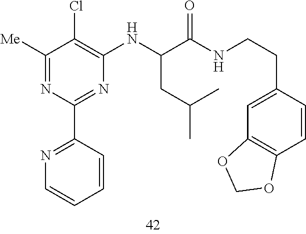

34. The compound of claim 1, of formula (V): 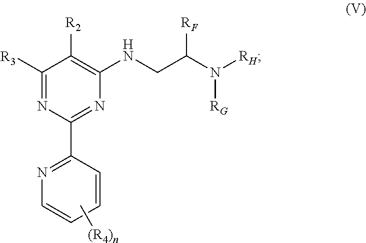
or a pharmaceutically acceptable salt thereof,
wherein,
R_{F} is an optionally substituted aryl, an optionally substituted heteroaryl, an optionally substituted cycloalkyl, or an optionally substituted heterocycloalkyl;
R_{G} is H or an optionally substituted alkyl;
R_{H} is X(CH₂)ₘY; and m is 1, 2, 3, 4, or 5;
X is C(O)ₚ, S(O)ₚ, or absent; and p is 0, 1, or 2;
Y is an optionally substituted cycloalkyl, an optionally substituted heterocycloalkyl, an optionally substituted aryl, or an optionally substituted heteroaryl;
R₂ is H, an optionally substituted alkyl, cyano, nitro, azido, or halo;
R₃ is an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;
each R₄ is independently an optionally substituted alkyl or hal; and
n is 0, 1, 2, 3, or 4.

35-44. (canceled)

45. The compound of claim 34, selected from:

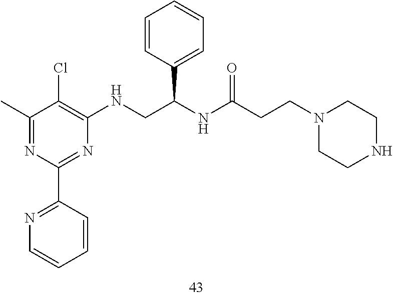

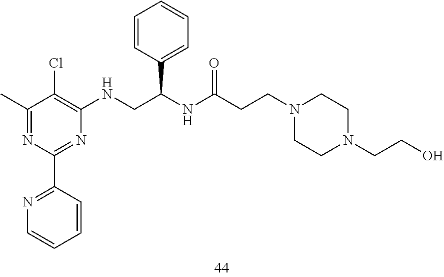

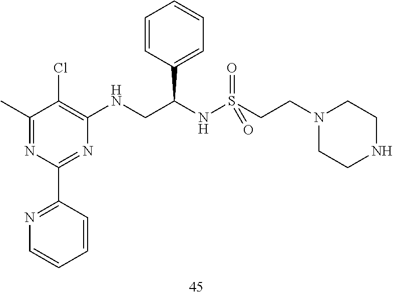

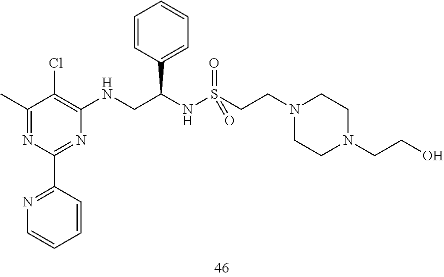

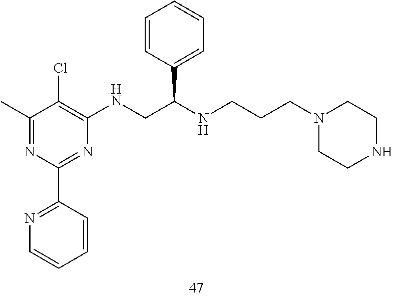

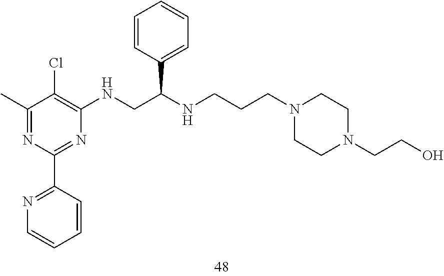

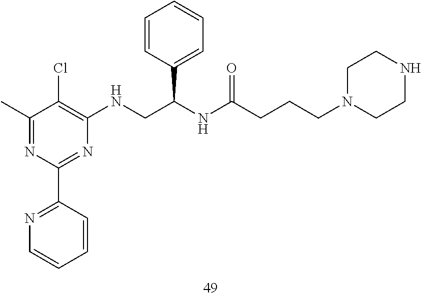

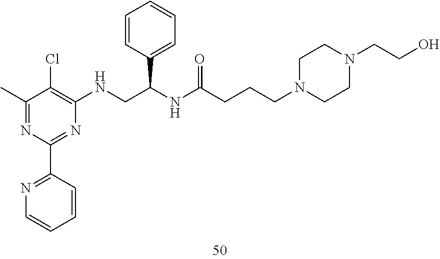

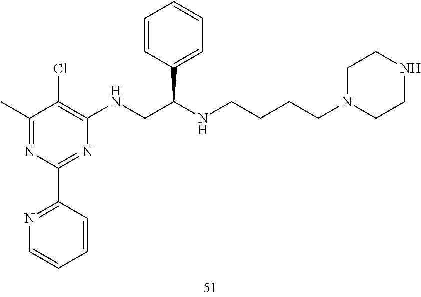

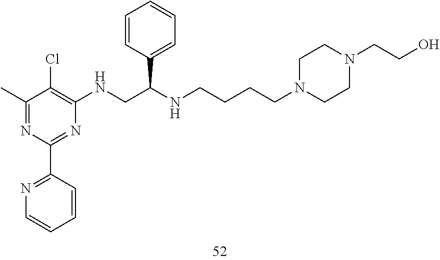

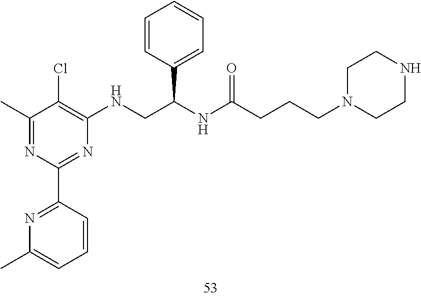

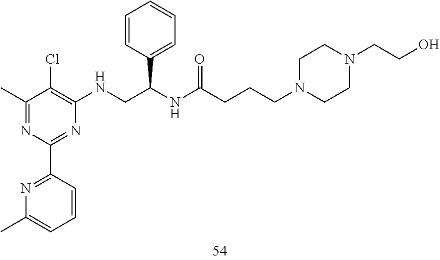

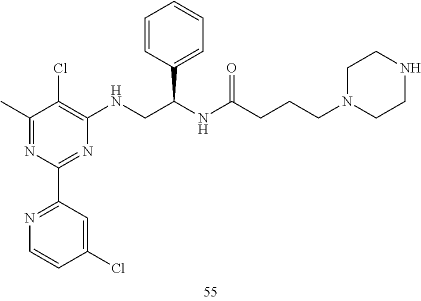

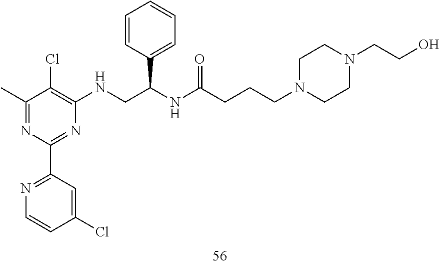

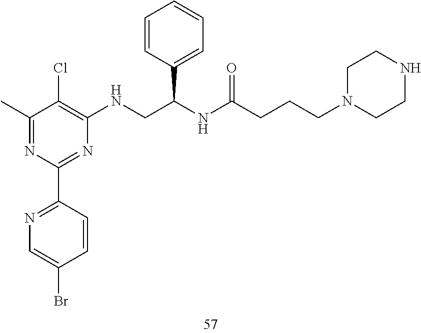

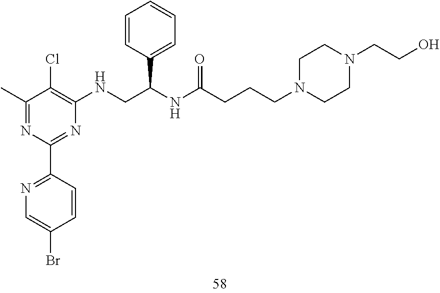

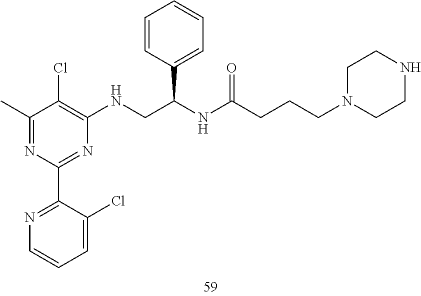

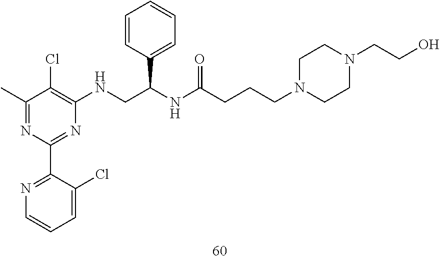

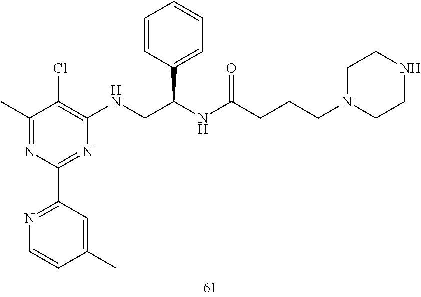

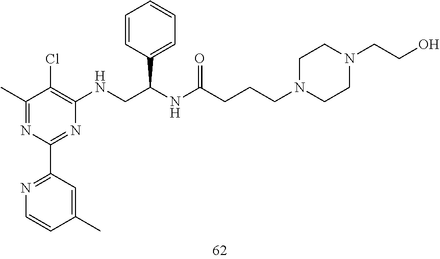

46. A compound selected from

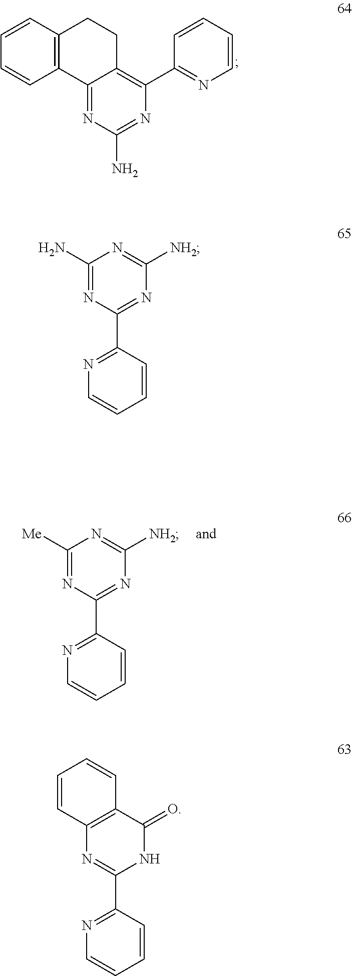

47. A composition comprising the compound of claim 1, and an additional therapeutic agent.

48-49. (canceled)

50. A method of treating a disease or disorder associated with methionine aminopeptidase in a subject, the method comprising the step of administering to the subject an effective amount of a compound of formula VI: 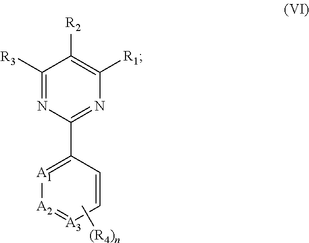
or a pharmaceutically acceptable salt thereof,
wherein,
any one of A₁, A₂, or A₃ is independently CH, CR₄, or N;
R₁ is NR_{A}R_{A}, NHR_{A}, OR_{A}, SR_{A}, optionally substituted heteroaryl, optionally substituted heterocyclic, optionally substituted cycloalkyl, or hal;
each R_{A} is independently H, an optionally substituted alkyl, an optionally substituted aryl, an optionally substituted aralkyl, an optionally substituted heteroaryl, or optionally substituted heterocyclic;
R₂ is H, an optionally substituted alkyl, an optionally substituted alkoxy, cyano, nitro, azido, or halo;
R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;
or R₂ and R₃ can be taken together to form an optionally substituted aryl;
each R₄ is independently an optionally substituted alkyl or hal; and
n is 0, 1, 2, 3, or 4.

51. The method of claim 50 further comprising identifying a subject identified as being in need of a hMetAP1 inhibitor and administering an effective amount of a compound of formula VI to the identified subject.

52. The method of claim 50, wherein the methionine aminopeptidase is human type 1 methionine aminopeptidase (hMetAP1).

53. The method of claim 52, wherein the disease or disorder associated with hMetAP1 is tumor, cancer growth, or neoplasia.

54-56. (canceled)

57. A method of modulating methionine aminopeptidase in a subject, the method comprising the step of administering to the subject an effective amount of a compound of formula VI: 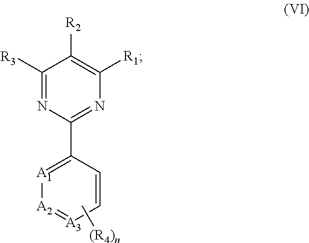
or a pharmaceutically acceptable salt thereof,
wherein,
any one of A₁, A₂, or A₃ is independently CH, CR₄, or N;
R₁ is NR_{A}R_{A}, NHR_{A}, OR_{A}, SR_{A}, optionally substituted heteroaryl, optionally substituted heterocyclic, optionally substituted cycloalkyl, or hal;
each R_{A} is independently H, an optionally substituted alkyl, an optionally substituted aryl, an optionally substituted aralkyl, an optionally substituted heteroaryl, or optionally substituted heterocyclic;
R₂ is H, an optionally substituted alkyl, an optionally substituted alkoxy, cyano, nitro, azido, or halo;
R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;
or R₂ and R₃ can be taken together to form an optionally substituted aryl;
each R₄ is independently an optionally substituted alkyl or hal; and
n is 0, 1, 2, 3, or 4.

58. The method of claim 57, wherein the methionine aminopeptidase is hMetAP1.

59-62. (canceled)

63. A method of treating tumor, cancer growth, or neoplasia in a subject, the method comprising the step of administering to the subject an effective amount of a compound of formula VI: 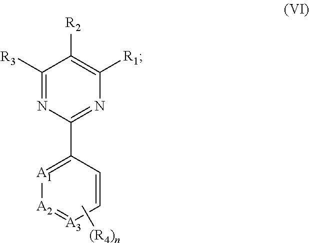
or a pharmaceutically acceptable salt thereof,
wherein,
any one of A₁, A₂, or A₃ is independently CH, CR₄, or N;
R₁ is NR_{A}R_{A}, NHR_{A}, OR_{A}, SR_{A}, optionally substituted heteroaryl, optionally substituted heterocyclic, optionally substituted cycloalkyl, or hal;
each R_{A} is independently H, an optionally substituted alkyl, an optionally substituted aryl, an optionally substituted aralkyl, an optionally substituted heteroaryl, or optionally substituted heterocyclic;
R₂ is H, an optionally substituted alkyl, an optionally substituted alkoxy, cyano, nitro, azido, or halo;
R₃ is H, an optionally substituted alkyl, an optionally substituted haloalkyl, an optionally substituted cycloalkyl, an optionally substituted heterocycloalkyl, an optionally substituted aryl, an optionally substituted heteroaryl, or an optionally substituted heteroaralkyl;
or R₂ and R₃ can be taken together to form an optionally substituted aryl;
each R₄ is independently an optionally substituted alkyl or hal; and
n is 0, 1, 2, 3, or 4;
wherein the compound inhibits hMetAP1 to thereby treat the tumor, cancer growth, or neoplasia.

64-73. (canceled)
